(19) United States
(12) Patent Application Publication
(10) Pub. No.: US 2026/0216134 A1
(43) Pub. Date: Jul. 30, 2026

(54) CHARGED PI-CONJUGATED MOLECULES FOR THE TREATMENT OF MICROBIAL INFECTIONS

(71) Applicants: William Marsh Rice University, Houston, TX (US); Fundació Institut d'Investigació Sanitària Illes Balears, Palma de Mallorca (ES)

(72) Inventors: James M. TOUR, Houston, TX (US); Ana L. SANTOS, Houston, TX (US); Bowen LI, Houston, TX (US); Gang LI, Houston, TX (US); Ciceron AYALA-OROZCO, Houston, TX (US); Tengda SI, Houston, TX (US)

(73) Assignees: William Marsh Rice University, Houston, TX (US); Fundació Institut d'Investigació Sanitària Illes Balears, Palma de Mallorca, IB (ES)

(21) Appl. No.: 19/145,839

(22) PCT Filed: Jan. 5, 2024
(86) PCT No.: PCT/US2024/010528

§ 371 Date: Jul. 3, 2025

Related U.S. Application Data
(60) Provisional application No. 63/437,303, filed on Jan. 5, 2023.

(51) Int. Cl. A61K 31/405; A61P 31/04

(52) U.S. Cl. CPC A61K 31/405; A61P 31/04

(57) ABSTRACT

In some aspects, the present disclosure provides methods of using pi-conjugated compounds that have a charge as part of that pi-conjugated structure to treat microbial infections. These methods may be used, in particular, to treat Gram-positive bacterial infections in a patient. In another aspect, the present disclosure provides methods of using pi-conjugated compounds that have a charge as part of that pi-conjugated structure to treat cancer.

### CROSS-REFERENCE TO RELATED APPLICATIONS

This application claims priority to U.S. Provisional Patent Application Ser. No. 63/437,303 filed Jan. 5, 2023, the entire contents of which are incorporated herein by reference.

### BACKGROUND

### 1. Field

This disclosure relates to the fields of biology, biochemistry, chemistry, pharmacology, and medicine. In particular, new methods, compounds, and methods of treating cancer or microbial infections are disclosed.

### 2. Related Art

In 2019, Staphylococcus aureus was the leading bacterial cause of death in 135 countries and was also associated with the most deaths in persons older than 15 years worldwide (Ikuta et al., 2019). Methicillin-resistant S. aureus (MRSA), in particular, was solely responsible for more than 100,000 deaths in 2019 alone (Murray et al., 2022). In the United States, MRSA is now responsible for more deaths than HIV/AIDS and tuberculosis combined (Peterson et al., 2010). In addition to the high mortality rate, MRSA infections cause an estimated $3.2 billion to $4.2 billion in additional health care costs annually (Bumah et al., 2013). Vancomycin has been a cornerstone of MRSA treatment for more than 20 years. However, in recent years, a number of vancomycin-intermediate Staphylococcus aureus infections (VISA) and vancomycin-resistant S. aureus (VRSA) infections have been reported (Weigel et al., 2003; Smith et al., 1999), jeopardizing the reliability of vancomycin in the treatment of MRSA.

Gram-positive vancomycin-resistant enterococci (VRE) are responsible for a variety of hospital-acquired infections, including urinary tract infections, skin/wound infections, intra-abdominal infections, and bacteremia. Among enterococci, Enterococcus faecalis and Enterococcus faecium are the third and fourth most common nosocomial pathogens in the world, and antibiotic resistance has developed and spread easily among these bacteria. Linezolid is generally the treatment of choice for VRE (Moellering et al., 2003), but it is expensive and can cause some serious side effects, including bone marrow suppression and peripheral neuropathy. It can also interact with psychiatric medications, particularly monoamine oxidase inhibitors. Alarmingly, linezolid resistance among VRE has also been reported (Gonzales et al., 2001).

Streptococcus is a genus of Gram-positive, facultatively anaerobic, coccoid bacteria that contains more than 50 species. It is found in a range of habitats, including human skin and mucous membranes. The epidemiology of Streptococcus is complex and can have serious consequences for human and animal health. In humans, it is one of the most frequent causes of bacterial infections, such as strep throat, sinusitis, meningitis, pneumonia, and skin infections. The species S. pneumoniae is the most lethal pathogen among children under 5 years old (Ikuta et al., 2019) while S. pyogenes, also known as Group A Streptococcus, is responsible for a wide range of illnesses, including strep throat, scarlet fever, rheumatic fever, and even necrotizing fasciitis (flesh-eating bacteria) in individuals of all age groups (Avire et al., 2021). In animals, streptococci are a major cause of mastitis in dairy cows, resulting in significant economic losses for the dairy industry. Streptococcus species are particularly prone to developing antibiotic resistance as a result of horizontal gene transfer and chromosomal point mutations that lead to altered efflux pump activity and antimicrobial target modifications.

There is, therefore, an urgent need for alternative antimicrobial therapies with modes of action distinct from those of existing antibiotics and with a low propensity for resistance development.

Cancer remains a leading worldwide cause of death, with approximately 20,000,000 cases and approximately 10,000,000 cases reported globally in 2020 (Xia et al., 2022). Therapeutic strategies which kill cancer cells with minimal toxic off-target effects are therefore urgently sought.

This invention was funded in part by the Robert A. Welch Foundation under Welch Grant No. C-2017.

### SUMMARY

As provided herein, the present disclosure relates to methods for treating bacterial infections comprising treatment with compounds with pi-conjugated compounds that have a charge as part of that pi-conjugated structure, provided at least part of the pi-conjugated structure is non-aromatic. These methods may be used, in particular, to treat Gram-positive bacterial infections in a patient. In some embodiments, the presently disclosed methods may be used to treat cancer in a patient. The charged pi-conjugated compounds may include but are not limited to cyanine dyes.

In some aspects, the present disclosure provides methods of killing bacteria by treatment with an extended conjugated compound of at least two double or triple bonds in conjugation that bears a charge in the conjugated chain. In some embodiments, the bacteria are Gram-positive bacteria. In other embodiments, the bacteria are Gram-negative bacteria. In another aspect, the present disclosure provides methods of killing a cancer cell by treatment with an extended conjugated compound of at least two double or triple bonds in conjugation that bears a charge in the conjugated chain. In another aspect, the present disclosure provides methods of killing a fungal cell by treatment with an extended conjugated compound of at least two double or triple bonds in conjugation that bears a charge in the conjugated chain.

In some embodiments, the methods further comprise treating the bacteria with a coadjuvant. In some embodiments, the coadjuvant is an antibiotic.

In some embodiments, the extended conjugated compound comprises a positive charge. In some embodiments, the conjugated compound is further defined by the formula:

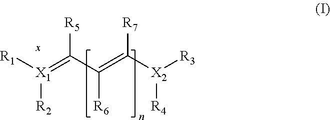

wherein:
x is a positive or negative charge;
n is an integer from 2 to 100; wherein the variables on each independent n is independently selected;
X₁ and X₂ are each independently a heteroatom selected from O, N, S, B, P, Ge, As, or Se; and
R₁, R₂, R₃, R₄, R₅, R₆, and R₇ are each independently hydrogen, alkyl_{(C≤18)}, alkenyl_{(C≤18)}, alkynyl_{(C≤18)}, aryl_{(C≤18)}, aralkyl_{(C≤18)}, heteroaryl_{(C≤18)}, heterocycloalkyl_{(C≤18)}, or a substituted version of any of these groups; or
R₁ and R₂, R₁ and R₅, R₂ and R₅, R₃ and R₄, R₃ and R₇, and R₄ and R₇ are taken together to form one, two, three, four, five, or six aliphatic or aromatic rings; comprising at least three carbon atoms and no more than 36 carbon atoms; optionally comprising one, two, three, four, or five nitrogen, sulfur, or oxygen atom.

In some embodiments, the compound is further defined as:

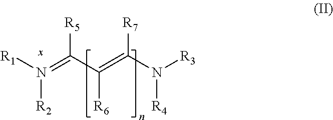

wherein:
x is a positive charge;
n is an integer from 0 to 100; wherein the variables on each independent n is independently selected;
each R₁, R₂, R₃, R₄, R₅, R₆, and R₇ are each independently hydrogen, alkyl_{(C≤18)}, alkenyl_{(C≤18)}, aryl_{(C≤18)}, alkynyl_{(C≤18)}, aralkyl_{(c≤18)}, heteroaryl_{(C≤18)}, heterocycloalkyl_{(C≤18)}, or a substituted version of any of these groups; or
each R₁, R₂, R₃, R₄, R₅, R₆, and R₇ are each independently a cell membrane targeting moiety, wherein the cell-targeting moiety optionally comprises a linker; or
each R₁ and R₂, R₁ and R₅, R₂ and R₅, R₃ and R₄, R₃ and R₇, R₄ and R₇, and R₅ and R₇ are taken together and each independently form one, two, three, four, five, or six aliphatic or aromatic rings; comprising at least three carbon atoms and no more than 36 carbon atoms; optionally comprising one, two, three, four, or five nitrogen, sulfur, or oxygen atom.

In some embodiments, X₁ and X₂ are identical. In some embodiments, X₁ is N. In some embodiments, X₂ is N. In some embodiments, X₁ and X₂ are N.

In some embodiments, R₁ or R₅ are symmetric with R₃ or R₄. In some embodiments, R₁ is taken together with R₅ to form one, two, three, four, or five rings. In some embodiments, R₁ is taken together with R₅ to form two, three, or four rings. In some embodiments, R₁ is taken together with R₅ to form three rings. In some embodiments, R₁ is taken together with R₅ to form three rings, wherein one ring is aliphatic and two rings are aromatic.

In some embodiments, R₂ is alkyl_{(C≤18)} or substituted alkyl_{(C≤18)}. In some embodiments, R₂ is alkyl_{(C≤18)}. In some embodiments, R₂ is alkyl_{(C-8)} such as methyl.

In some embodiments, R₃ is taken together with Ry to form one, two, three, four, or five rings. In some embodiments, R₃ is taken together with R₇ to form two, three, or four rings. In some embodiments, R₃ is taken together with R₇ to form three rings. In some embodiments, R₃ is taken together with R₇ to form three rings, wherein one ring is aliphatic and two rings are aromatic.

In some embodiments, R₄ is alkyl_{(C≤18)} or substituted alkyl_{(C≤18)}. In some embodiments, R₄ is alkyl_{(C≤18)}. In some embodiments, R₄ is alkyl_{(C≤8)} such as methyl. In some embodiments, R₆ is hydrogen.

In some embodiments, R₅ and R₇ are taken together and form one, two, or three rings. In some embodiments, R₅ and R₇ are taken together and form a single ring. In some embodiments, the single ring is a five, six, or seven-membered ring. In some embodiments, n is an integer from 1 to 10. In some embodiments, n is an integer selected from 2, 3, 4, or 5. In some embodiments, n is 3.

In some embodiments, R₄ is a cell targeting moiety with a linker. In some embodiments, the linker is an alkyl chain, an alkenyl chain, an aryl chain, a peptide chain, a polyethylene glycol chain, or a polypropylene chain. In some embodiments, the linker further comprises one or more joining functional group selected from ether, amide, disulfide, ester, amine, or thioether. In some embodiments, the linker is two alkyl chains with an amide joining functional group. In some embodiments, the cell-targeting moiety is a functional group that associates with the membrane, a carbohydrate or polysaccharide that binds to one or more markers on the membrane, a lipid that binds to one or more markers on the cell membrane, a small molecule that binds to one or more markers on the cell membrane, an aptamer that binds to one or more markers on the membrane, or a peptide or an antibody that binds to one or more markers on the membrane. In some embodiments, the cell-targeting moiety is a functional group that associates with the cell membrane. In some embodiments, the functional group that associates with the cell membrane is an amine. In some embodiments, the amine is protonated.

In some embodiments, the compound is further defined as a compound as described in FIG. 1. In some embodiments, the compound is selected from among the following compounds:

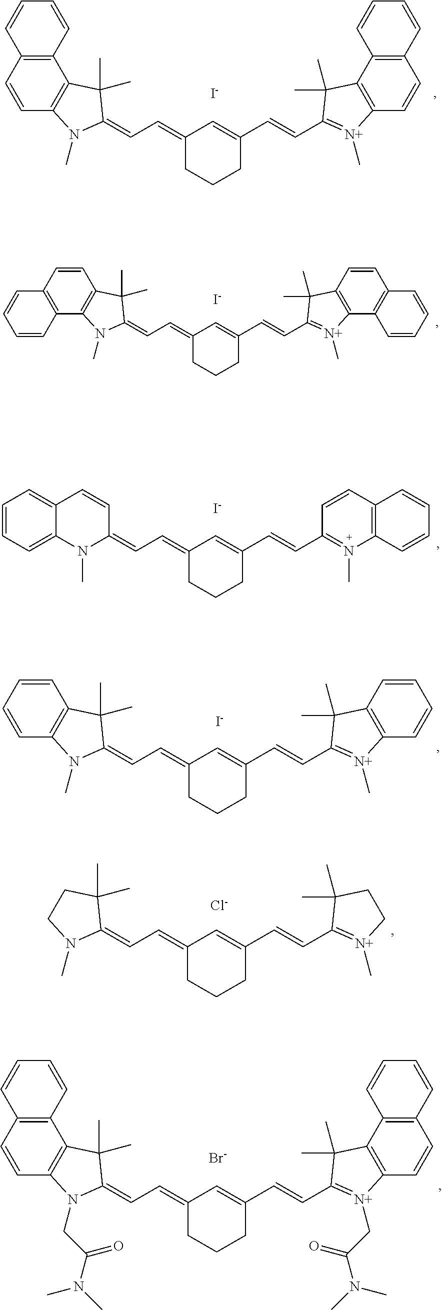

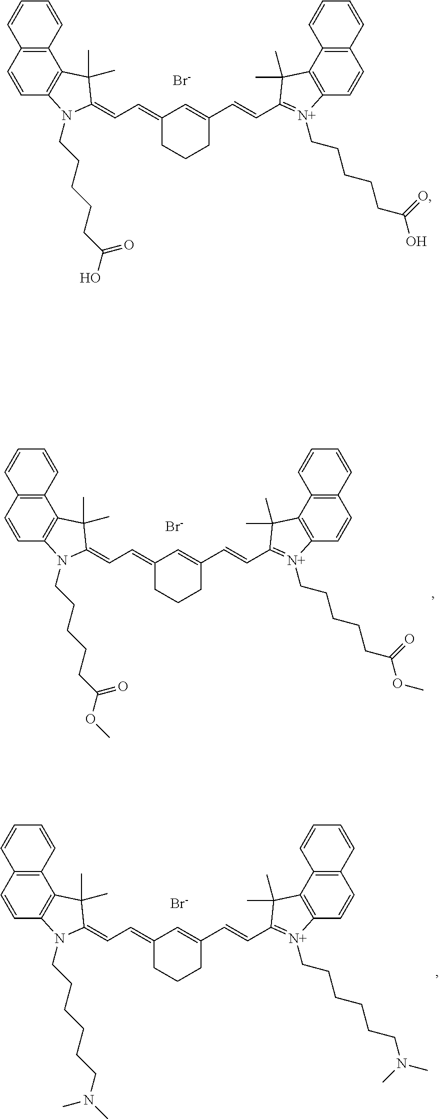 [SMILES: CC1(C)C(/C=C/C2=CC(=C/C=C3/N(CCCCCC(=O)O)c4ccc5ccccc5c4C3(C)C)/CCC2)=[N+](CCCCCC(=O)O)c2ccc3ccccc3c21 ; [Br-] ; COC(=O)CCCCCN1/C(=C/C=C2C=C(/C=C/C3=[N+](CCCCCC(=O)OC)c4ccc5ccccc5c4C3(C)C)CCC/2)C(C)(C)c2c1ccc1ccccc21 ; [Br-] ; CN(C)CCCCCCN1/C(=C/C=C2C=C(/C=C/C3=[N+](CCCCCCN(C)C)c4ccc5ccccc5c4C3(C)C)CCC/2)C(C)(C)c2c1ccc1ccccc21 ; [Br-]]

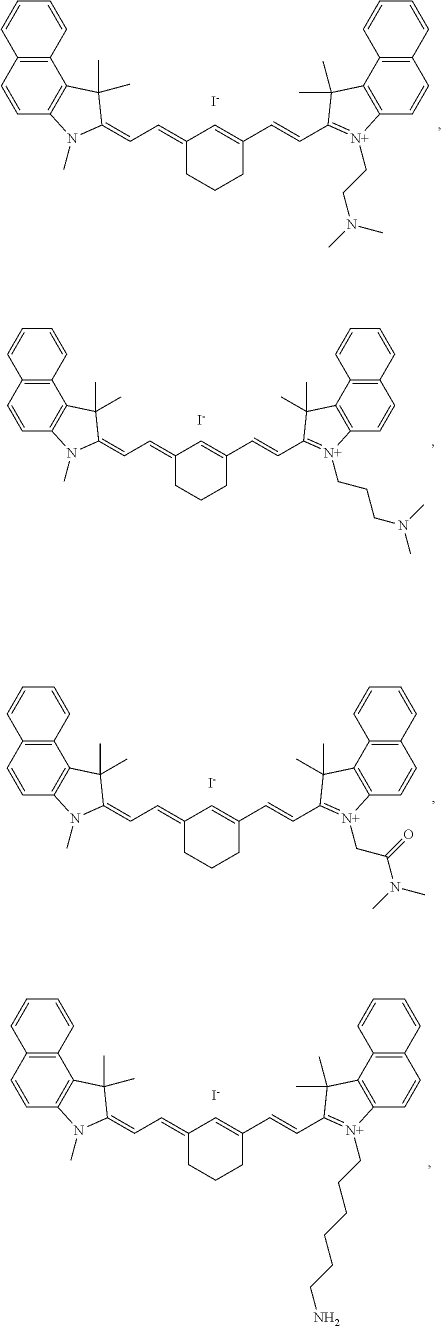

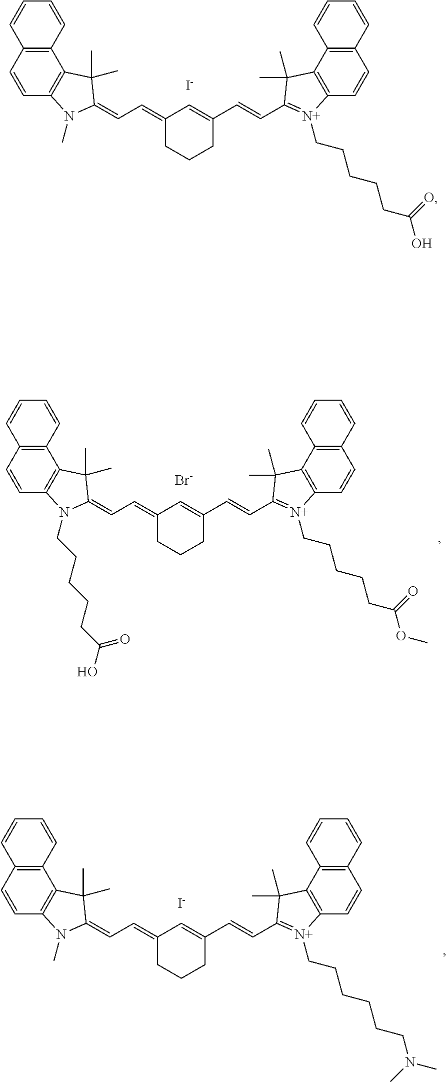 [SMILES: CN1/C(=C/C=C2C=C(/C=C/C3=[N+](CCCCCC(=O)O)c4ccc5ccccc5c4C3(C)C)CCC/2)C(C)(C)c2c1ccc1ccccc21 ; [I-] ; COC(=O)CCCCC[N+]1=C(/C=C/C2=CC(=C/C=C3/N(CCCCCC(=O)O)c4ccc5ccccc5c4C3(C)C)/CCC2)C(C)(C)c2c1ccc1ccccc21 ; [Br-] ; CN(C)CCCCCC[N+]1=C(/C=C/C2=CC(=C/C=C3/N(C)c4ccc5ccccc5c4C3(C)C)/CCC2)C(C)(C)c2c1ccc1ccccc21 ; [I-]]

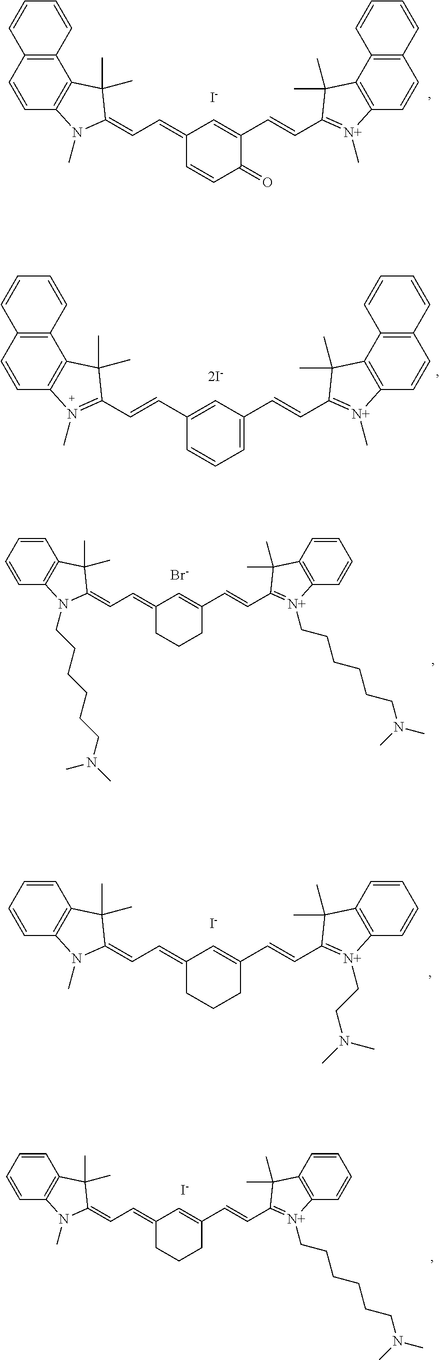

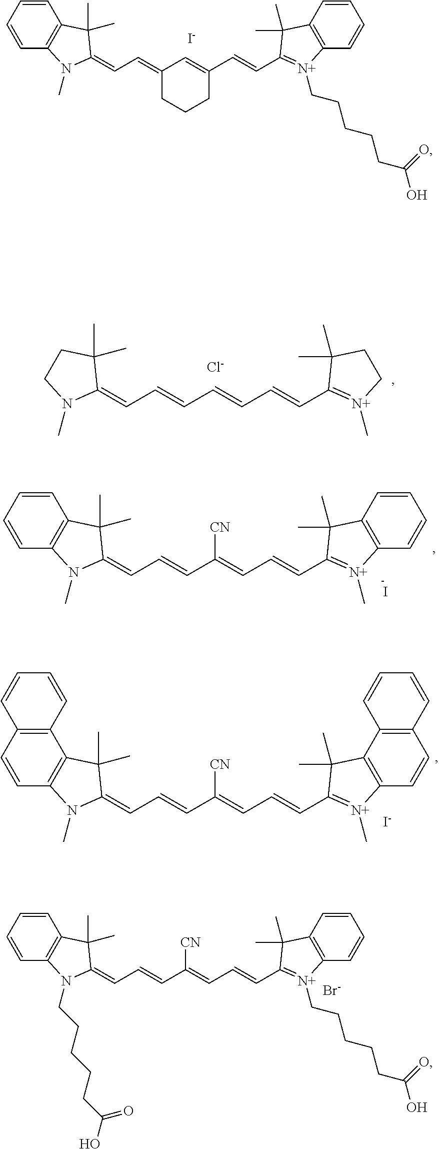

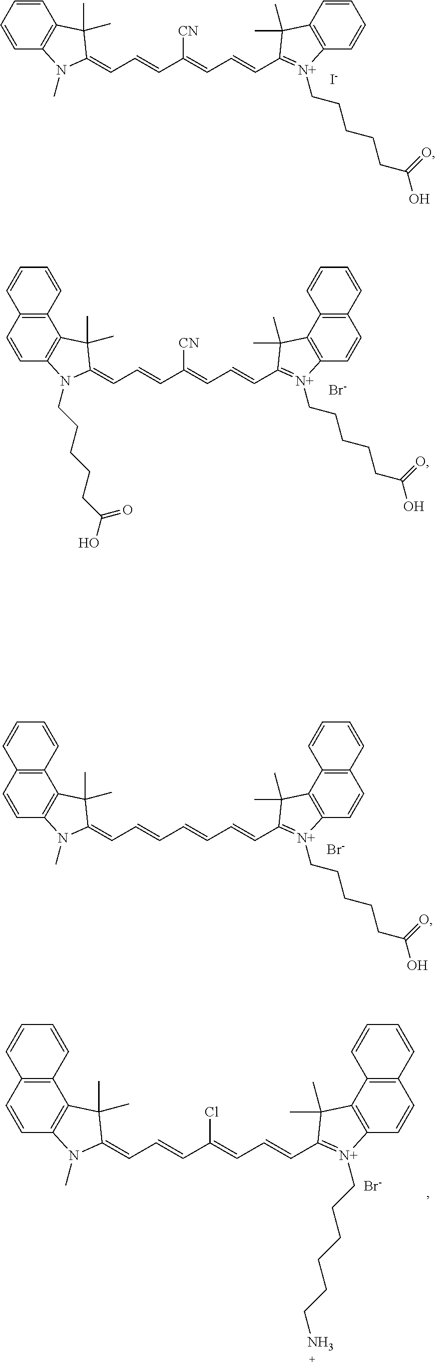

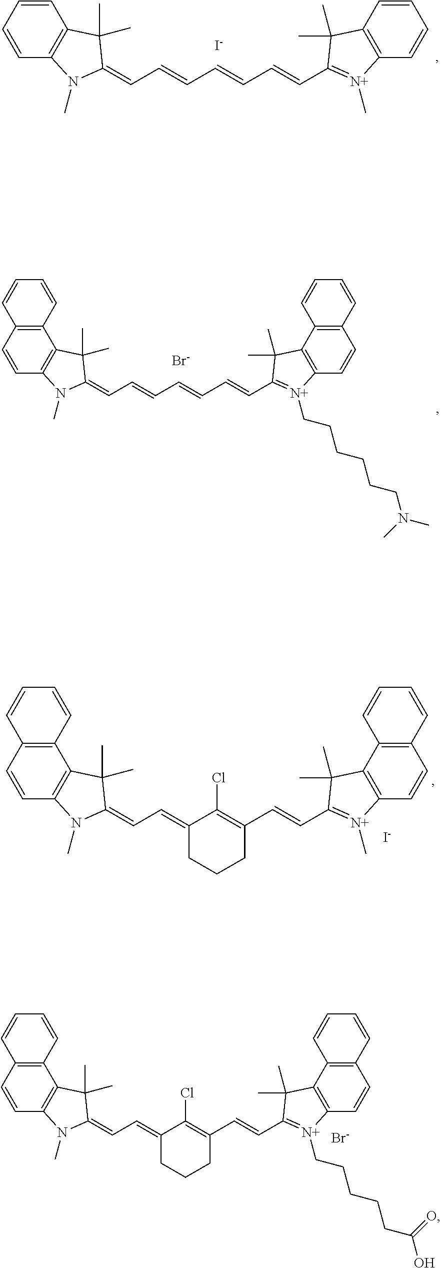

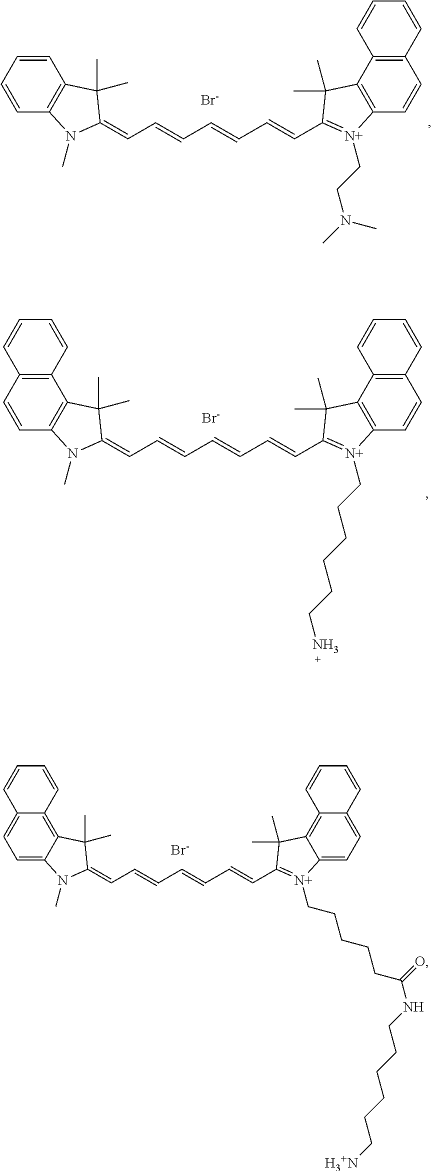

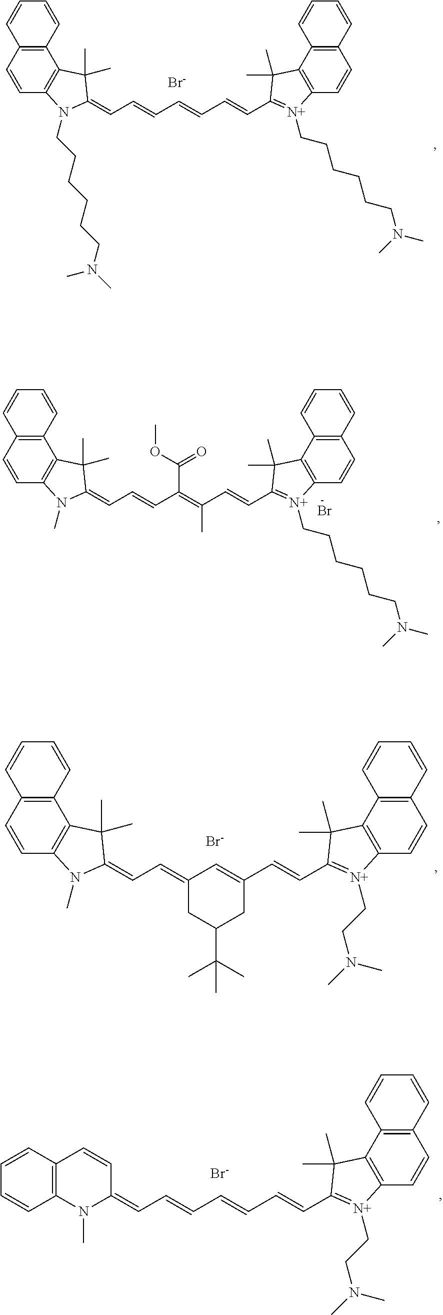

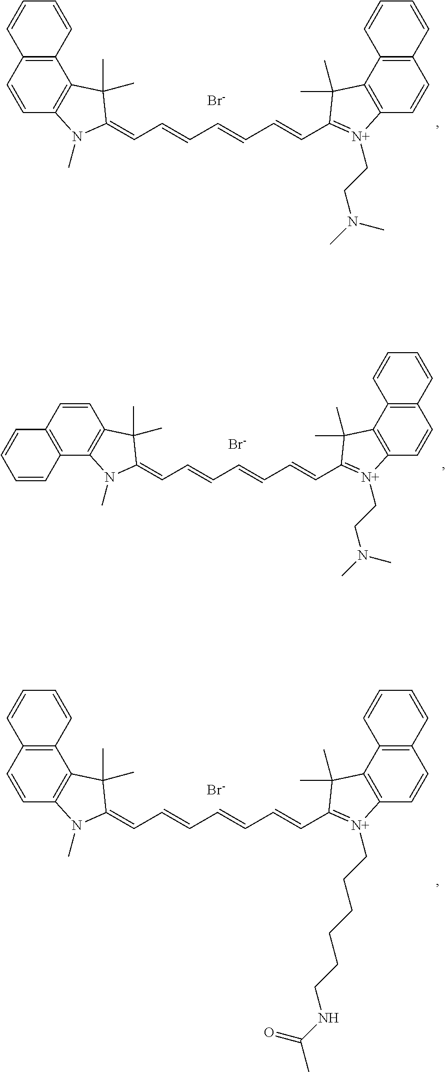 [SMILES: CN(C)CC[N+]1=C(/C=C/C=C/C=C/C=C2/N(C)c3ccc4ccccc4c3C2(C)C)C(C)(C)c2c1ccc1ccccc21 ; [Br-] ; CN(C)CC[N+]1=C(/C=C/C=C/C=C/C=C2/N(C)c3c(ccc4ccccc34)C2(C)C)C(C)(C)c2c1ccc1ccccc21 ; [Br-] ; CC(=O)NCCCCCC[N+]1=C(/C=C/C=C/C=C/C=C2/N(C)c3ccc4ccccc4c3C2(C)C)C(C)(C)c2c1ccc1ccccc21 ; [Br-]]

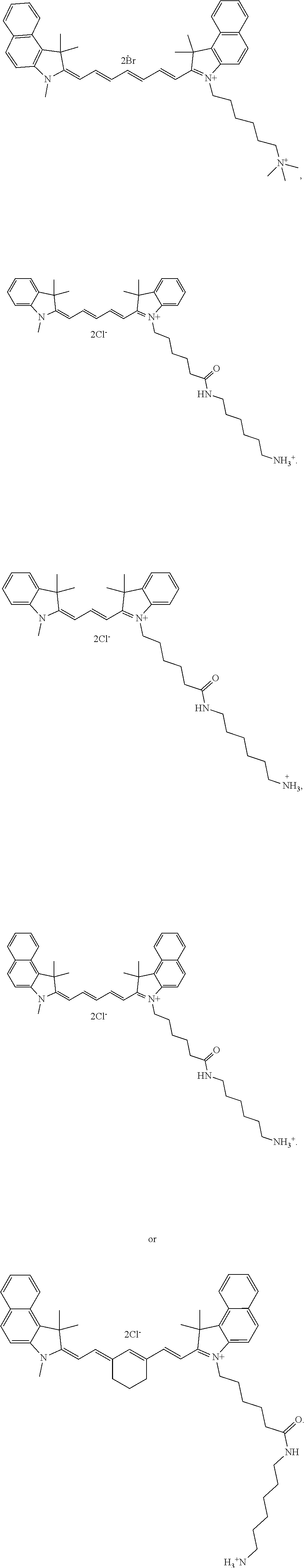

In some embodiments, the compound is selected from among the following compounds:

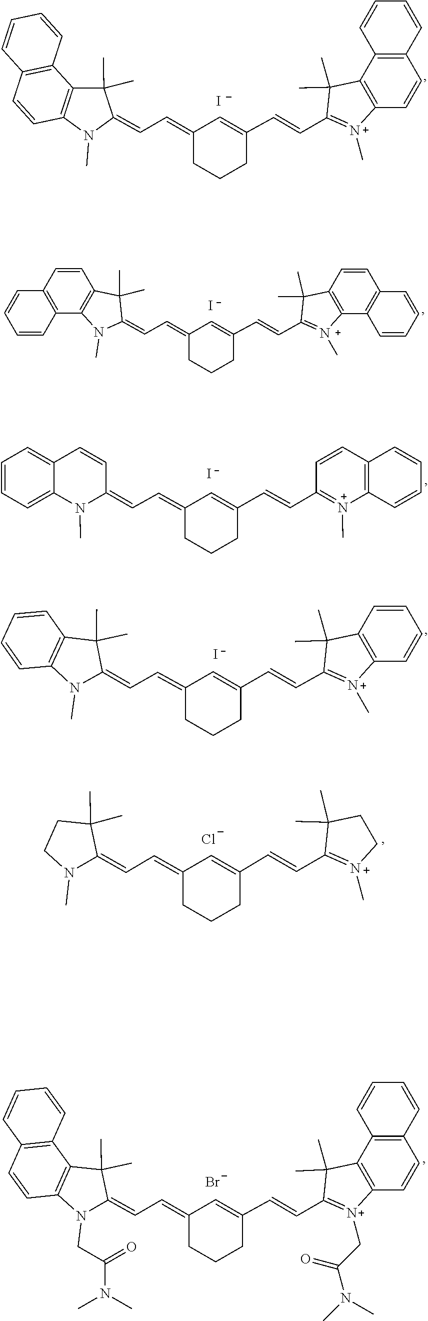

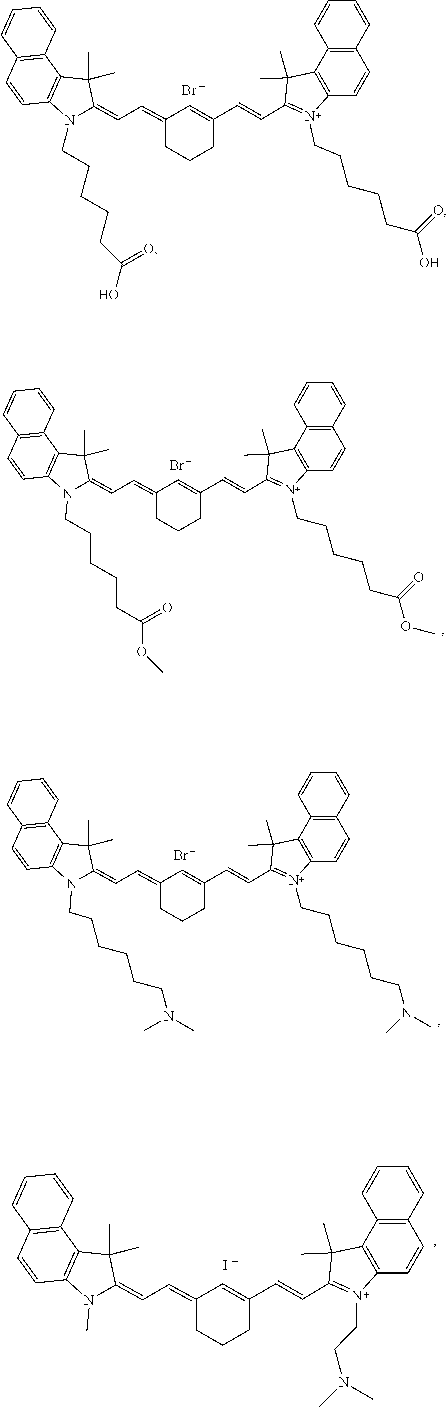

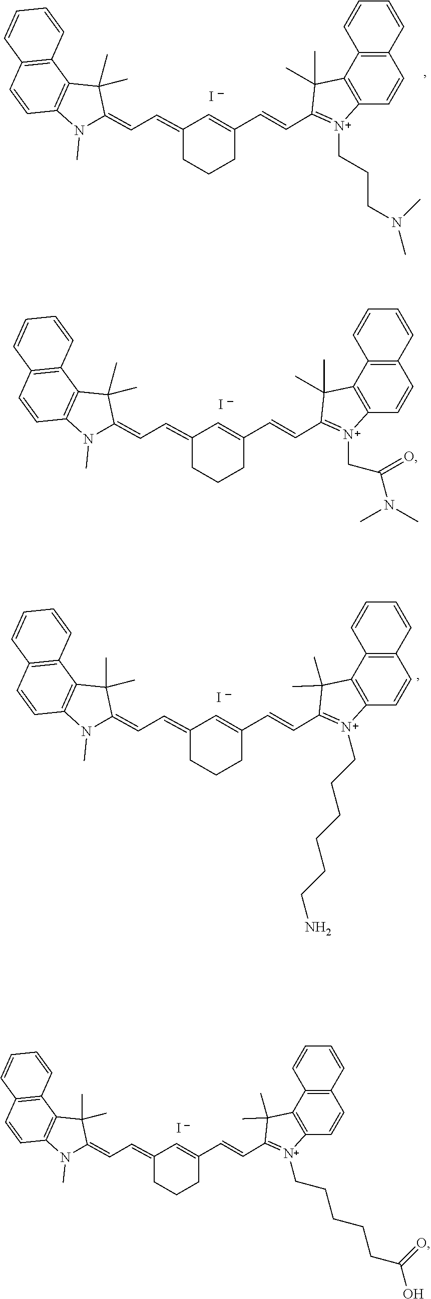

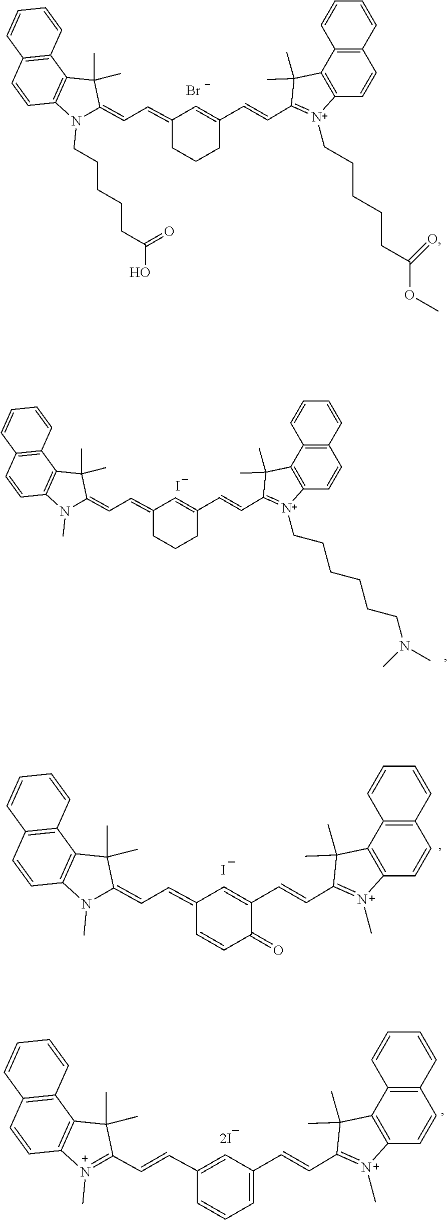

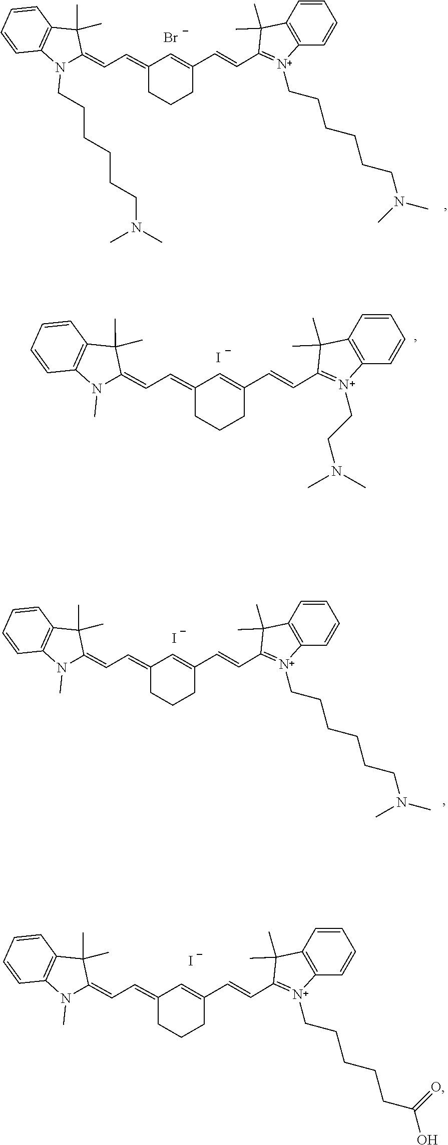

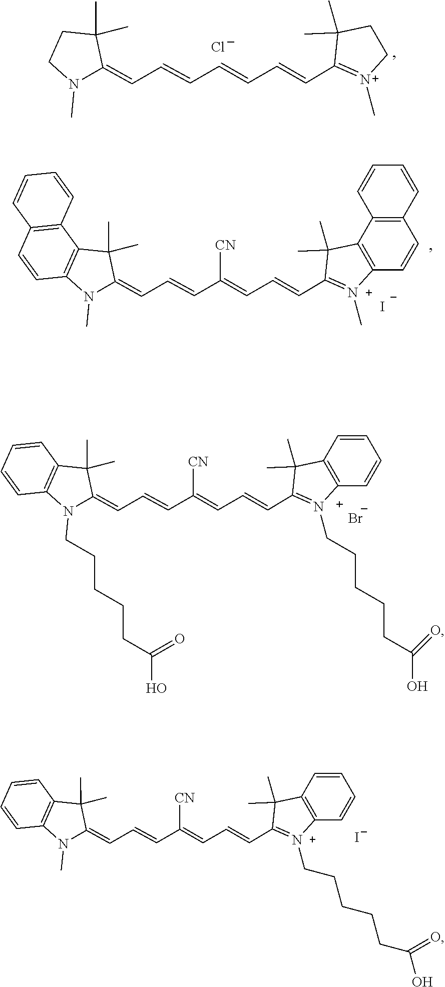

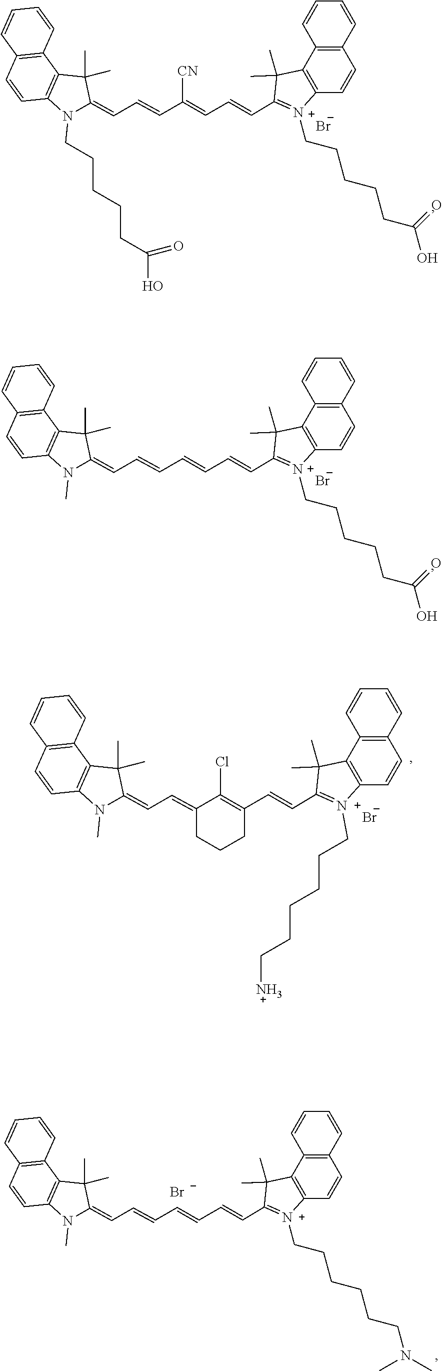

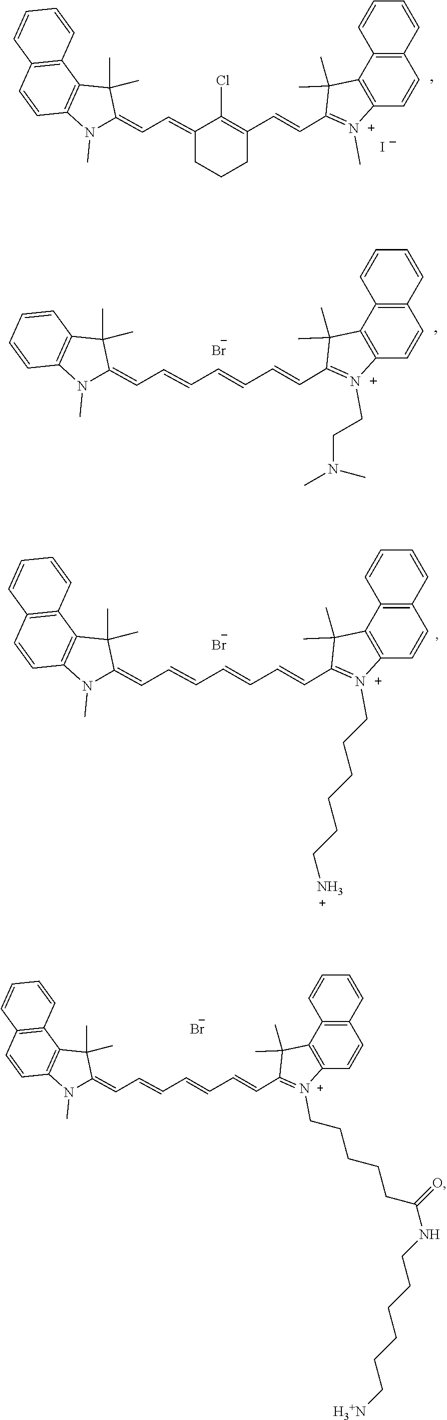

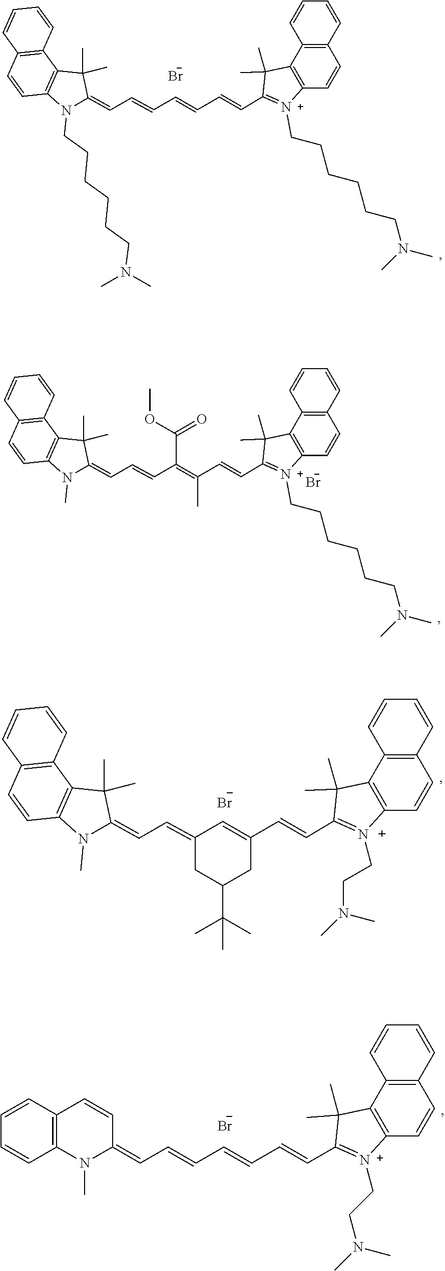

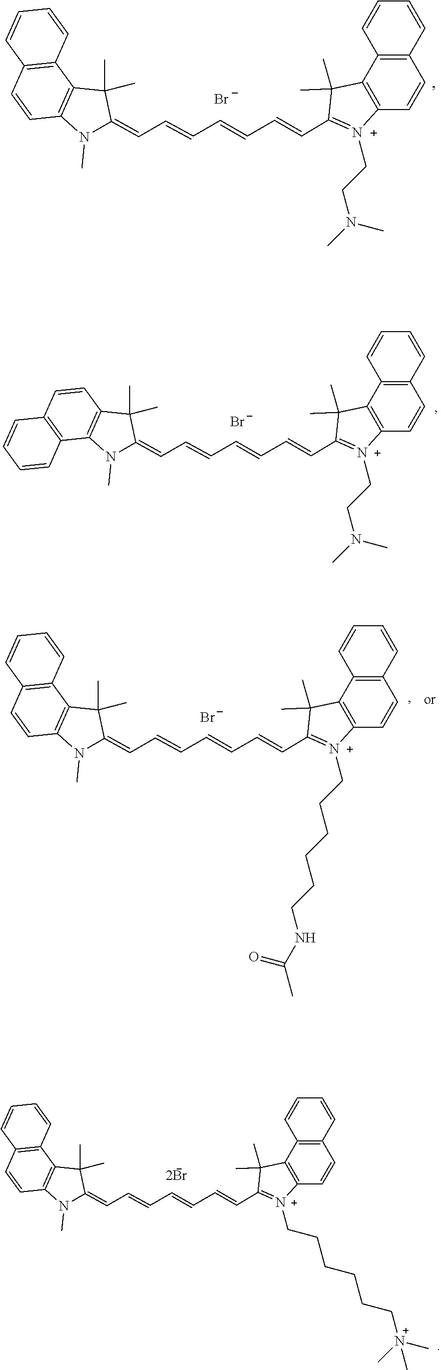

In some embodiments, the compound is selected from among the following group of compounds:

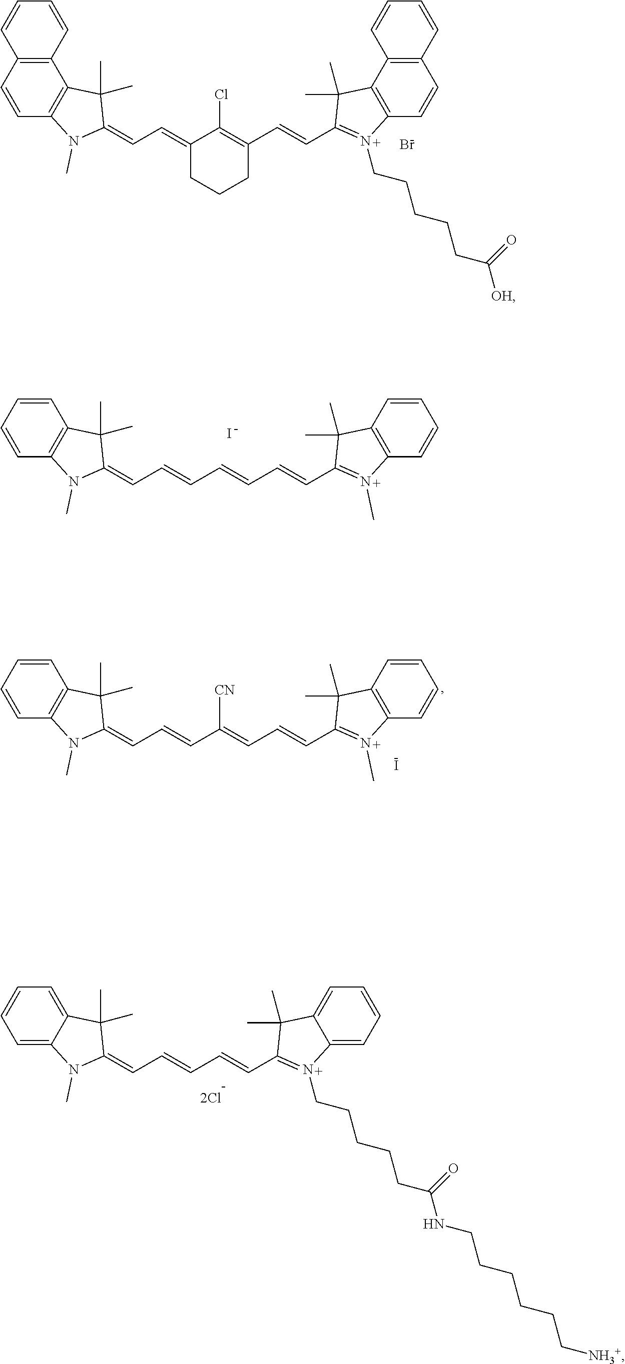

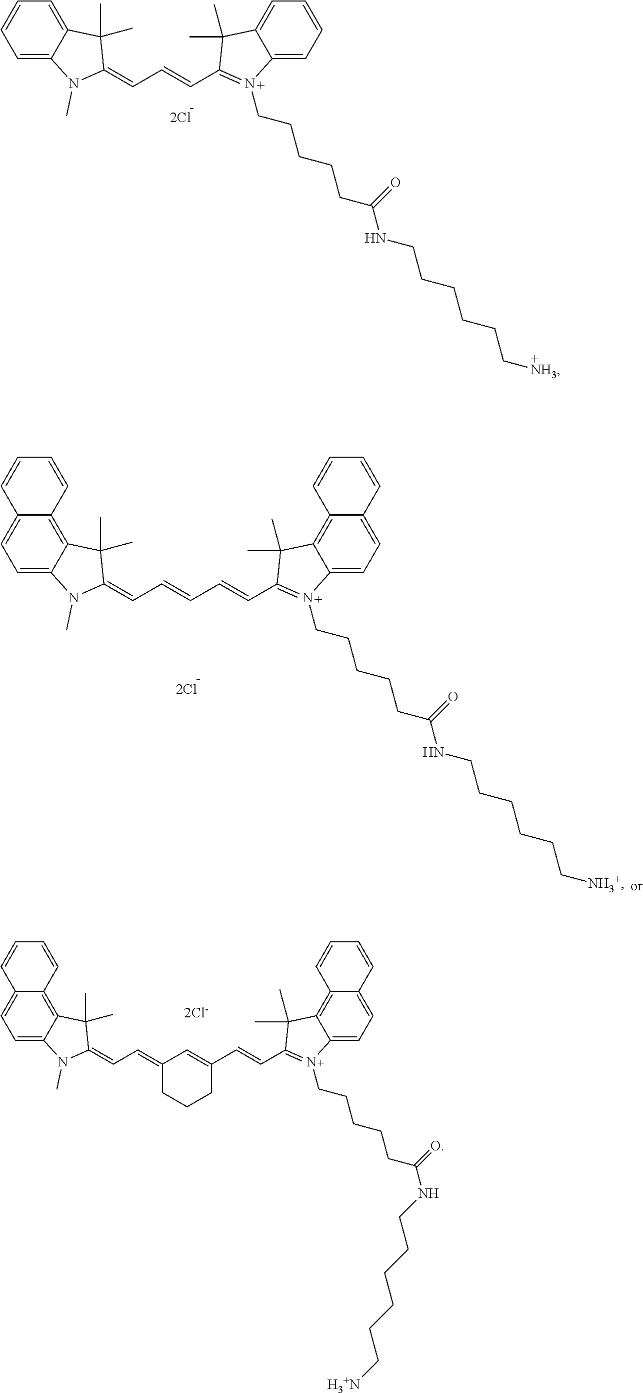

In some embodiments, the compound is further defined as:

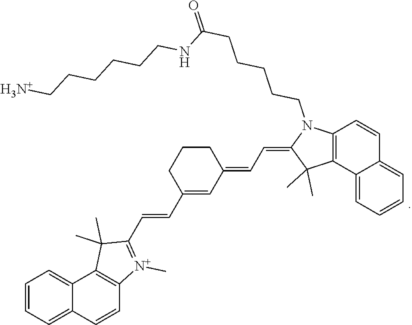 [SMILES: C[N+]1=C(/C=C/C2=CC(=C/C=C3/N(CCCCCC(=O)NCCCCCC[NH3+])c4ccc5ccccc5c4C3(C)C)/CCC2)C(C)(C)c2c1ccc1ccccc21]

In still yet another aspect, the present disclosure provides methods of treating a bacterial disease or disorder in a patient comprising treatment with an extended conjugated compound of at least two double or triple bonds in conjugation that bears a charge in the conjugated chain. In some embodiments, the methods further comprise administering the compound with a different antibiotic. In some embodiments, the extended conjugated compound is sufficient to treat or prevent the disease or disorder. In some embodiments, the compound is further defined as the compound as described herein. In some embodiments, the patient is a mammal such as a human.

In yet another aspect, the present disclosure provides methods of killing viruses by treatment with an extended conjugated compound of at least two double or triple bonds in conjugation that bears a charge in the conjugated chain.

In still another aspect, the present disclosure provides methods of killing fungi by treatment with an extended conjugated compound of at least two double or triple bonds in conjugation that bears a charge in the conjugated chain.

In another aspect, the present disclosure provides methods of killing parasites by treatment with an extended conjugated compound of at least two double or triple bonds in conjugation that bears a charge in the conjugated chain.

In some embodiments, the compound used in the methods have a structure as described herein. In some embodiments, the methods further comprise a second therapeutic agent. In some embodiments, the methods are sufficient to treat or prevent a disease or disorder caused by either cancer, a virus, a fungus, or a parasite.

In another aspect, the present disclosure provides methods of forming a compound of formula
(IV):

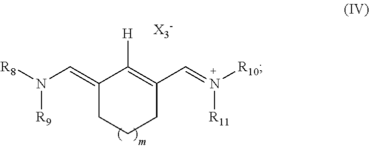

wherein:
   m is 0, 1, 2, or 3;
   X₃ is Br, Cl, I, or F; and
   R₈, R₉, R₁₀, and R₁₁ are each independently hydrogen, alkyl_{(C≤18)}, alkenyl_{(C≤18)}, alkynyl_{(C≤18)}, aryl_{(C≤18)}, aralkyl_{(C≤18)}, heteroaryl_{(C≤18)}, heterocycloalkyl_{(C≤18)}, or a substituted version of any of these groups;
      comprising the steps of:
(a) obtaining a compound of formula (III)

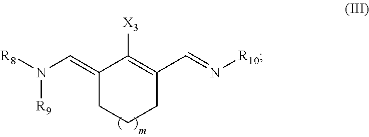

wherein:
   m is 0, 1, 2, or 3;
   X₃ is Br, Cl, I, or F; and
   R₈, R₉, and R₁₀ are each independently hydrogen, alkyl_{(C≤18)}, alkenyl_{(C≤18)}, alkynyl_{(C≤18)}, aryl_{(C≤18)}, aralkyl_{(C≤18)}, heteroaryl_{(C≤18)}, heterocycloalkyl_{(C≤18)}, or a substituted version of any of these groups; and
(b) reacting the compound of formula (III) with a catalyst and a base under conditions sufficient to form the compound of formula (IV).

It is contemplated that any method or composition described herein can be implemented with respect to any other method or composition described herein. For example, a compound synthesized by one method may be used in the preparation of a final compound according to a different method.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features, and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the chemical structures of representative charged pi-conjugated compounds for which antibacterial activity is presented herein.

FIGS. 2A-2D provide evidence that charged pi-conjugated compounds are light-independent, Gram-positive-specific, narrow-spectrum antibacterial agents. (FIG. 2A) Illustrative chemical structure of a charged pi-conjugated compound, with “R” representing variable functional groups. (FIG. 2B) Concentration-dependent growth inhibition (%, assessed as OD₆₀₀) of a representative Gram-positive bacterium (S. aureus ATCC 25923) and a representative Gram-negative bacterium (E. coli μMG1655) in the presence and absence of 48 J cm-2 of 730 nm light. Data represent the mean of at least three biological replicates±standard error of the mean. (FIG. 2C) Minimum inhibitory concentration (MIC) values of different charged pi-conjugated compounds in E. coli and S. aureus. Arrows next to bars indicate MIC values exceeding the maximum tested concentration (80 μM). Dots denote individual data points. (FIG. 2D) Growth curves (OD₆₀₀) of representative Gram-positive strains S. aureus ATCC 25923 and E. faecalis ATCC 29212 treated with increasing concentrations of a representative charged pi-conjugated compound. The results are shown as the average (solid lines) of three replicates±standard error of the mean (shaded area).

FIG. 3 provides further evidence that charged pi-conjugated compounds do not exhibit bacteriostatic potential against Gram-negative bacteria. In addition to E. coli (FIG. 2B, right; FIG. 2C, right), charged pi-conjugated compounds exhibited no bacteriostatic activity against the Gram-negative bacteria Pseudomonas aeruginosa and Acinetobacter baumannii. The bars represent results from at least three biological replicates. The arrows next to the bars indicate that the MIC value was higher than the maximum concentration tested (80 μM).

FIG. 4 shows the susceptibility of different Gram-positive bacterial strains, including antibiotic-resistant strains, to a panel of 56 compounds of the present disclosure. Susceptibility was assessed using the minimum inhibitory concentration (MIC). Horizontal bars indicate average MIC values computed from at least three biological replicates. Dots denote individual data points. Instances where the MIC exceeded the highest tested compound concentration are indicated by arrows. Lower MIC values correspond to increased antibacterial activity.

FIGS. 5A-5B summarize data related to the antibacterial activity of charged pi-conjugated compounds against Gram-positive bacteria. (FIG. 5A) MIC₅₀ values (the concentration at which 50% of the strains are inhibited) of 56 different charged pi-conjugated compounds against 126 bacterial strains (FIG. 1, Table 2). Each bar indicates the average MIC₅₀ value for a given charged pi-conjugated compound, whereas individual dots above the bars represent individual MIC₅₀ data points for each bacterial strain studied. (FIG. 5B) Average MIC₅₀ values sorted by bacterial genus. Bars show the average MIC₅₀ values for all strains within a given genus, with overlaid dots representing individual MIC data points for the examined strains within that genus.

FIGS. 6A-6F provide evidence that charged pi-conjugated compounds display potent bactericidal activity against exponentially growing Gram-positive bacteria. (FIG. 6A) Time-kill plots for Gram-positive bacterial strains in the exponential phase after treatment with different charged pi-conjugated compounds and standard antibiotics. The graph shows bacterial survival over time after treatment with different charged pi-conjugated compounds at 4×MIC compared with the standard antibiotics linezolid (LNZ) and vancomycin (VAN). Survival is presented as log(N/N0), where N is the bacterial count at a given time point, and NO is the initial bacterial count. Data points indicate the average bacterial survival for each time point, with error bars reflecting the standard error of the mean (SEM). The dashed line indicates the detection limit, below which the number of bacteria cannot be accurately quantified. (FIG. 6B) shows the impact of increasing concentrations of charged pi-conjugated compounds on the growth (optical density at 600 nm) of MRSA USA300. (FIG. 6C) shows the impact of increasing concentrations of charged pi-conjugated compounds on the growth (optical density at 600 nm) of a representative vancomycin-resistant enterococci (VRE) strain. (FIG. 6D) Time-kill curves of different Gram-positive strains treated with 4×MIC of different charged pi-conjugated compounds. Survival is presented as log(N/N0), where N is the bacterial count at a given time point, and NO is the initial bacterial count. The dashed line denotes the limit of detection of the method. (FIG. 6E) Time-kill curves of different Gram-positive strains treated with 8×MIC of different charged pi-conjugated compounds. Survival is presented as log(N/N0), where N is the bacterial count at a given time point, and NO is the initial bacterial count. The dashed line denotes the limit of detection of the method. (FIG. 6F) shows the time-kill curves of different Gram-positive strains treated with 16×MIC of different charged pi-conjugated compounds. Survival is presented as log(N/N0), where N is the bacterial count at a given time point, and NO is the initial bacterial count. The dashed line denotes the limit of detection of the method.

FIG. 7 provides evidence that charged pi-conjugated compounds effectively eradicate antibiotic-tolerant persister cells. Time-kill plots of persister cells of different Gram-positive bacterial strains treated with different charged pi-conjugated compounds and conventional antibiotics. The graphs show bacterial survival over time after treatment with different charged pi-conjugated compounds at 4× their respective MICs compared to standard antibiotics, linezolid (LNZ), and vancomycin (VAN). Survival is presented as log(N/N₀), where N is the bacterial count at a given time point, and N₀ is the initial bacterial count. Data points reflect the average bacterial survival at each time point, with error bars indicating the standard error of the mean (SEM). The dashed line marks the detection limit, below which the number of bacteria cannot be accurately quantified.

FIG. 8 provides evidence that charged pi-conjugated compounds display variable antibiofilm activity. The efficacy of various charged pi-conjugated compounds at 8×MIC against established biofilms of various Gram-positive strains is shown. The bars show the changes in biofilm biomass measured by the crystal violet assay, indicating the density and adhesion of the biofilm matrix. The superimposed lines show the changes in colony-forming units (CFU), which reveal changes in the number of viable bacteria in the treated biofilms. The results represent the average of three independent replicates, with error bars indicating the standard error of the mean (SEM). Asterisks denote the significance of differences between charged pi-conjugated compounds-treated samples and DMSO-treated samples. *P<0.05, **P<0.01, ***P<0.001, ****P<0.0001.

FIG. 9 shows the antibiofilm activity of charged pi-conjugated compounds or the vehicle in different Gram-positive bacterial strains assessed by quantifying the percentage of metabolically active cells determined from biofilm ATP levels. The results are shown as the average±SEM. Dots are individual data points. Error bars represent the standard deviation. Asterisks denote the significance of differences between charged pi-conjugated compounds-treated samples and DMSO-treated samples. *P<0.05, **P<0.01, ***P<0.001, ****P<0.0001.

FIGS. 10A-10C show the development of resistance and mutation rate of bacterial strains subjected to repeated exposure to charged pi-conjugated compounds. (FIG. 10A) Stepwise development of resistance in B. cereus CECT131123, E. faecalis ATCC 29212, S. aureus ATCC 25923, and S. pneumoniae ATCC 49619. Bacterial strains were repeatedly exposed to charged pi-conjugated compounds or the conventional antibiotics ciprofloxacin (CIP), linezolid (LNZ) and vancomycin (VAN) for over 30 cycles. The y-axis indicates the MIC fold change, reflecting increased resistance over repeated treatment. (FIG. 10B) Mutation rate, represented as the frequency of rifampin-resistant cells, in S. aureus ATCC 25923 subjected to repeated cycles of treatment with charged pi-conjugated compounds or the conventional antibiotic ciprofloxacin (CIP). (FIG. 10C) Single-step resistance assessment of spontaneous mutants. Overnight cultures of S. aureus ATCC 25923 were plated on media containing increasing concentrations of charged pi-conjugated compounds (4×, 8×, or 16×MIC). The table shows the frequency of spontaneous mutants emerging in the presence of the respective charged pi-conjugated compound concentrations, expressed as the ratio of the number of resistant colony-forming units (CFUs) to the total number of CFUs.

FIGS. 11A-11F show the influence of various physicochemical parameters on the susceptibility of S. aureus ATCC 25923 and E. faecalis ATCC 29212 to selected charged pi-conjugated compounds. (FIG. 11A) Time-kill curves of S. aureus and E. faecalis treated with 4× MIC of different charged pi-conjugated compounds at 25° C. and 37° C. (FIG. 11B) MIC values of different charged pi-conjugated compounds against S. aureus and E. faecalis across a pH gradient. (FIG. 11C) Variation in MIC values in the presence of increasing serum concentrations. (FIG. 11D) Effect of varying NaCl concentrations on the susceptibility of the bacterial strains to charged pi-conjugated compounds. (FIG. 11E) Influence of Ca²⁺ concentration on MIC values against both bacterial strains. (FIG. 11F) MIC values in relation to initial bacterial inoculum size, expressed as optical density at 600 nm (OD₆₀₀).

FIGS. 12A-12E show transcriptional changes elicited by treatment with sublethal concentrations of the representative charged pi-conjugated compound BL 248 in S. aureus assessed using RNA sequencing (RNA-seq). (FIG. 12A) Heatmap displaying the expression patterns of transcripts showing the most significant changes upon treatment with BL 248 compared to the DMSO vehicle. The color gradient represents log-transformed expression values, with red indicating upregulation and blue indicating downregulation. (FIG. 12B) Principal Component Analysis (PCA) plot illustrating the clustering patterns of samples treated with BL 248 and DMSO. Principal Component 1 (PC1) explained 94% of the total variance. Each dot represents an individual sample, with teal representing DMSO-treated samples and coral representing BL 248-treated samples. (FIG. 12C) Volcano plot showing the distribution of differentially expressed genes between BL 248 and DMSO treatments. The x-axis represents the log 2-fold change in gene expression, whereas the y-axis displays the log₁₀ transformed p-values. Genes with significant changes in expression are highlighted and labeled. (FIG. 12D) Bar chart depicting the enriched Gene Ontology (GO) biological processes among the transcripts showing the most significant changes upon BL 248 treatment. The x-axis represents the fold enrichment of each biological process, with biological processes considered significantly enriched at a false discovery rate (FDR) of P<0.05. (FIG. 12E) Protein-protein interaction (PPI) network of proteins encoded by transcripts with the most significant changes upon BL 248 treatment. Nodes represent proteins, and edges denote the known interactions between them. Node colors differentiate between proteins associated with the proton-transporting ATP synthase complex (purple), the plasma membrane (blue), and other membranes (green).

FIG. 13 shows the effect of increasing concentrations of various cyanine dyes or the vehicle (1% DMSO) on intracellular, extracellular, and total ATP levels normalized to protein levels in S. aureus. The results are shown as the mean of three biological replicates±standard error of the mean (SEM).

FIG. 14 shows the changes in the membrane potential of S. aureus after treatment with varying concentrations of charged pi-conjugated compounds, or the vehicle control (DMSO) determined using the voltage-sensitive dye DiBAC4 (5). Each plotted curve represents the mean fluorescence value from multiple experiments, and the shaded area around each curve indicates the standard error of the mean (SEM). An increase in DiBAC4 (5) fluorescence is associated with the depolarization of the bacterial membrane, whereas a decrease indicates hyperpolarization.

FIGS. 15A-15C provide data related to the interactions between charged pi-conjugated compounds and conventional antibiotics. (FIG. 15A) Representative checkerboard plates showing the interaction between different charged pi-conjugated compounds and conventional antibiotics with different mechanisms of action. The corresponding Fractional Inhibitory Concentration Index (FICI) is shown in red in the box. The results are shown as a heatmap, with white denoting no growth (0%) and blue denoting growth (100%). Growth was assessed as the absorbance at 600 nm. The results are the average of three independent replicates. FICI values of ≤0.5 denote synergistic interactions, values>0.5 but≤4.0 denote indifferent (or additive) interactions, and values>4.0 denote antagonistic interactions, respectively. (FIG. 15B) Summary plot depicting the FICI for the interactions between charged pi-conjugated compounds and different antibiotics. The results are shown as the average±standard deviation. The dots denote individual values. (FIG. 15C) Time-kill curves of S. aureus treated with 0.5×MIC of individual charged pi-conjugated compounds, 0.5×MIC of antibiotics (gramicidin, GRAMI, or daptomycin, DAP), or a combination of 0.5×MIC charged pi-conjugated compound and 0.5× MIC antibiotic. Survival is presented as log(N/N₀), where N is the bacterial count at a given time point, and N₀ is the initial bacterial count. Data points reflect the average bacterial survival at each time point, with error bars indicating the standard error of the mean (SEM). The dashed line marks the detection limit, below which the number of bacteria cannot be accurately quantified.

FIGS. 16A-16B provide data related to the interaction between different charged pi-conjugated compounds. (FIG. 16A) Representative checkerboard plates showing the interaction between different charged pi-conjugated compounds. The corresponding FICI is shown in red in the box. The results are shown as a heatmap, with white denoting no growth (0%) and blue denoting growth (100%). The results are the average of three independent replicates. (FIG. 16B) Time-kill curves of S. aureus treated with 0.5×MIC of individual charged pi-conjugated compounds or a combination of 0.5×MIC of different charged pi-conjugated compounds. Survival is presented as log(N/N₀), where N is the bacterial count at a given time point, and No is the initial bacterial count. Data points reflect the average bacterial survival at each time point, with error bars indicating the standard error of the mean (SEM). The dashed line marks the detection limit, below which the number of bacteria cannot be accurately quantified.

FIGS. 17A-17B demonstrate the in vitro biocompatibility of charged pi-conjugated compounds with mammalian cell lines. (FIG. 17A) Dose-response curves showing the effect of increasing concentrations of charged pi-conjugated compounds on the viability of two mammalian cell lines (A549, HEK293) assessed using the Presto Blue assay. The data represent the average of at least three independent biological replicates #standard error of the mean (SEM). Solid lines represent the sigmoidal dose-response curve fit for each compound. (FIG. 17B) IC₅₀, i.e., the concentration resulting in a 50% viability reduction of different charged pi-conjugated compounds calculated from dose-response curves and the therapeutic index (TI) calculated from the ratio of the average IC₅₀ for the two cell lines tested and the MIC of each charged pi-conjugated compound in MRSA USA300.

FIG. 18A-18D show the impact of increasing concentrations of charged pi-conjugated compounds on the viability of the different mammalian cell lines assessed using the clonogenic assay. (FIG. 18A) Survival of A549 mammalian cells treated with increasing concentrations of different charged pi-conjugated compounds as assessed with the clonogenic assay. (FIG. 18B) Survival of HEK-Blue™ hNOD1 mammalian cells treated with increasing concentrations of different charged pi-conjugated compounds as assessed with the clonogenic assay. (FIG. 18C) Survival of HEK-Blue™ TLR mammalian cells treated with increasing concentrations of different charged pi-conjugated compounds as assessed with the clonogenic assay. (FIG. 18D) Survival of HepG2 mammalian cells treated with increasing concentrations of different charged pi-conjugated compounds as assessed with the clonogenic assay.

FIG. 19 provides dose-dependent curves of different mammalian cell lines (MH-S, HEK-Blue™ hNOD1, A549) treated with increasing concentrations of different charged pi-conjugated compounds. Survival was assessed by quantifying ATP levels using a luminescence-based assay. Dots denote individual data points. Solid lines represent the sigmoidal dose-response curve fit for each compound.

FIG. 20 allows evaluation of charged pi-conjugated compound biocompatibility in vivo, assessed by monitoring the survival of Galleria mellonella (n=9 individuals) after injection with increasing concentrations of charged pi-conjugated compound or the vehicle.

FIG. 21 provides survival curves of G. mellonella (n=5 individuals) after MRSA infection and subsequent treatment with increasing concentrations of charged pi-conjugated compounds. Uninfected worms and worms treated with the vehicle were also used as controls.

FIGS. 22A-22C show structures that are particularly relevant to the experiments described in Example 3. (FIG. 22A) Exemplary, non-limiting design of structure that can be utilized for cancer treatment. Where Z is a non-carbon atom, “x” is a charge such as cation, anion, radical cation, radical anion, or radical. R1, R2, R3, R4 can be alkyl to alkyl or aryl to aryl substituents. R1-R7 can be H, alkyl, aryl, annulated rings that are aliphatic or aromatic, etc. For cancer treatment, it is important that at least one of the pendant R groups have a moiety that can promote cell-association, such as an amine that will be protonated at physiological pH, which will prefer lipid bilayer association-thereby driving the dye into the lipid bilayer. Alternatively, a lipid, carbohydrate, polysaccharide, peptide, or antibody may be added to promote selective cell-association or even cell entry. (FIG. 22B) More particularly, the charged pi-conjugated compounds of the present disclosure may be cyanine dyes, of which a general structure is provided in FIG. 23B. R1, R2, R3, R4 can be alkyl to alkyl or aryl to aryl substituents. R1-R7 can be H, alkyl, aryl, annulated rings that are aliphatic or aromatic, etc. For the effect described in Example 3, it is important that at least one of the pendant R groups have a moiety that can promote cell-association. Such as an amine that will be protonated at physiological pH, which will prefer lipid bilayer association-thereby driving the dye into the lipid bilayer. (FIG. 22C) Structure of Cyanine 7.5-amine, also referred to herein as Cy7.5-amine, with a protonated amine as in physiological pH. This compound has a pendant ammonium ion for good cell association as described in FIG. 22A and FIG. 22B.

FIG. 23 shows the charged pi-conjugated compounds studied in the experiments described in Example 3.

FIGS. 24A-24F show the effect of contact time in the cytotoxicity of charged pi-conjugated compounds using crystal violet test. The contact time during the incubation of charged pi-conjugated compounds molecules with A375 cells has a strong effect on the cytotoxicity. The cell viability was measured by crystal violet test. (FIG. 24A) Cytotoxicity of molecule Cy7.5-amine at 40 min vs 1 day. (FIG. 24B) Cytotoxicity of molecule Cy5.5-amine at 40 min vs 2 days. (FIG. 24C) Cytotoxicity of molecule GL-261-2 at 50 min vs 2 days. (FIG. 24D) Cytotoxicity of molecule GL-286 at 1 day vs 2 days. (FIG. 24E) Cytotoxicity of molecule BL-142 at 1 day vs 2 days. (FIG. 24F) Cytotoxicity of molecule GL-291-2 at 1 day vs 2 days. Notice that the mild effects at short contact time (~40-50 min) are cytotoxic effects through slow a mechanism, likely apoptosis, since this was measured after exposing the cells for 40-50 min and then 2 days of cell culture. Each data point was quadruplicated in the crystal violet analysis (n=4). The error bars are the standard deviation.

FIG. 25 shows crystal violet tests to calculate the toxicity IC₅₀ without light activation and with long contact time (2 days of incubation). The inhibitory concentration of charged pi-conjugated compounds needed to inhibit the cell growth by 50% (toxicity IC₅₀).

FIG. 26 shows crystal violet tests to calculate the toxicity IC₅₀ without light activation and with long contact time (2 days of incubation). The inhibitory concentration of charged pi-conjugated compounds needed to inhibit the cell growth by 50% (toxicity IC₅₀).

FIG. 27 summarizes the data shown in FIG. 25 and FIG. 26 collected to determine the cytotoxicity of charged pi-conjugated compounds without light activation. The inhibitory concentration of charged pi-conjugated compounds needed to inhibit the cell growth by 50% (toxicity IC₅₀).

FIGS. 28A-28B provide a summary of the evidence of the cytotoxicity of charged pi-conjugated compounds without light. Toxicity was measured using clonogenic assay with 7 hours of incubation (contact time between molecules and A375 cells) and 7 days for colony formation. (FIG. 28A) The lethal concentration of the cyanine molecules to kill 50% of the A375 cells (Tox LC₅₀) without light activation. (FIG. 28B) Chemical structure of exemplary charged pi-conjugated compounds. On average, 300 cells equal to 300 colonies were seeded and analyzed for each data point.

FIG. 29 shows the data demonstrating cytotoxicity of charged pi-conjugated compounds without light. Both properties were measured using clonogenic assay with 7 hours of incubation (contact time between molecules and A375 cells) and 7 days for colony formation. 300 cells were seeded and analyzed for each data point.

FIGS. 30A-30G provide pictures of clonogenic assay for toxicity activity (without light). Toxicity was measured using clonogenic assay with 7 h of incubation (contact time between molecules and A375 cells) and 7 days for colony formation. (FIG. 30A) Molecule GL-261-2. (FIG. 30B) Molecule GL-286. (FIG. 30C) Molecule Cy7-amine. (FIG. 30D) Molecule GL-297-2. (FIG. 30E) Molecule BL-141-2. (FIG. 30F) Molecule BL-204. (FIG. 30G) Molecule BL-141-1. On average, 300 cells were seeded and analyzed in each plate.

FIG. 31A-31D provide pictures of clonogenic assay for toxicity activity (without light). Toxicity was measured using clonogenic assay with 7 h of incubation (contact time between molecules and A375 cells) and 7 days for colony formation. (FIG. 31A) Molecule BL-242. (FIG. 31B) Molecule BL-362-2. (FIG. 31C) Molecule BL-246-1. (FIG. 31D) Molecule BL-142. On average, 300 cells were seeded and analyzed in each plate.

FIG. 32 provides evidence of toxicity of charged pi-conjugated compounds on HEMa (normal melanocytes) versus on A375 melanoma using 1 day of incubation versus 2 days of incubation. BL-204, BL-141-2, BL-304 are aminocyanines. In contrast, TS-1-32 and GL-261-2 are two names for a carboxycyanine compound shown as GL-261-2 in FIG. 23.

FIG. 33 shows the treatment schedule to A375 tumors. Cy7.5-amine was intratumorally injected at the dose of 8 μg (equivalent to 50 μL of 200 μM Cy7.5-amine solution in PBS containing 2.5% DMSO. As control 50 μL of 2.5% DMSO in PBS solution was injected intratumorally. The blue dots indicate the days when the tumors were treated.

FIGS. 34A-34D provide evidence that Cy7.5-amine causes loss of A375 tumors in mice. (FIG. 34A) The intratumoral injection of the control 2.5% DMSO in PBS solution does not cause tumor loss. 10 mice were injected. (FIG. 34B) The intratumoral injection of 200 μM Cy7.5-amine in PBS solution containing 2.5% DMSO has an effect on the loss of tumors (n=10 mice). (FIG. 34C) The intratumoral injection of 200 μM Cy7.5-amine has an effect on the survival on the mice with A375 tumors (n=10 mice). Still after 7 months, 2 out of 10 mice were tumor-free. (FIG. 34D) The Cy7.5-amine is safe to the mice. Cy7.5-amine does not cause change of the mice body weight relative to the control group injected with 2.5% DMSO. There is not loss of body weight during the treatments.

### DESCRIPTION OF ILLUSTRATIVE EMBODIMENTS

The present disclosure relates to the use of several charged pi-conjugated compounds with potent antibacterial activity, specifically against Gram-positive bacteria, including MRSA, VRE, linezolid-resistant S. epidermidis, and Streptococcus strains. These charged pi-conjugated compounds may be further cyanine dyes. This is the first systematic investigation of the antibacterial activity of a library of different charged pi-conjugated compounds. The present studies identified several molecules with potent antibacterial activity via a novel mechanism of action against a broad range of Gram-positive bacterial pathogens, including MRSA, VRE, and Streptococcus strains. The molecules were found to be not only bacteriostatic but also bactericidal. The presently described charged pi-conjugated compounds were also found to impair the growth of bacteria, extending the lag phase of the growth curve and reducing total bacterial biomass. Cells treated with the present charged pi-conjugated compounds displayed a transient increase in ATP levels at sublethal concentrations, followed by a strong decrease in ATP levels at concentrations above the MIC, denoting an impact of the present charged pi-conjugated compounds on microbial metabolism. Treatment with the present charged pi-conjugated compounds also resulted in a concentration-dependent reduction in membrane potential. For some Gram-positive bacterial strains, treatment with the present charged pi-conjugated compounds also resulted in a significant reduction in biofilm viability and biomass. Pre-treatment of cells with the present charged pi-conjugated compounds also potentiated the activity of the conventional antibiotic rifampin.

Biocompatibility assessment in different mammalian cell lines identified several candidate molecules with a therapeutic index equal or superior to (Murray et al., 2022). In vivo toxicity assessment in an invertebrate model revealed overall good biocompatibility. These and more details are described herein.

In another aspect, the present disclosure relates to the use of several charged pi-conjugated compounds with potent anticancer activity. These charged pi-conjugated compounds may be further cyanine dyes. In some embodiments, the direct injection of charged pi-conjugated compounds results in direct and efficient cell death, shrinking of the tumor and final removal by biological mechanisms of cell death and clearance. In some embodiments, the charged pi-conjugated compounds as used herein may be advantageous as chemotherapeutic drugs that do not require addition of targeting addends, such as for example peptides for the specific recognition of cancer cells. In some embodiments, the pi-conjugated charged compounds are highly toxic to cancer cells and safe to surrounding healthy tissue or cells. Therefore, the charged pi-conjugated compounds may be useful as chemotherapeutic drugs or for systemic treatment at lower concentrations than used for similar compounds in therapies known in the art. Moreover, the charged pi-conjugated compounds of the present disclosure have a distinct pharmacological mode of action, which would not be expected based on the known uses of such compounds. The presently disclosed use of charged pi-conjugated compounds for the treatment of cancer without photoactivation or generation of reactive oxygen species (ROS) represents a contribution over the known art. Details on these aspects and more are described in the sections that follow.

### I. COMPOUNDS AND FORMULATIONS THEREOF

### A. Compounds

The compounds of the present disclosure are shown, for example, above, in the summary of the invention section, the Examples section, and in the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

All the charged pi-conjugated compounds of the present disclosure may, in some embodiments, be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such, unless explicitly stated to the contrary, all the charged pi-conjugated compounds of the present disclosure are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting public health by assuring the safety, effectiveness, quality, and security of human and veterinary drugs, vaccines and other biological products, and medical devices.

The present charged pi-conjugated compounds may be used in an amount from about 100 nM to about 10 mM, from about 250 nM to about 5 mM, or from about 500 nM to about 2 mM. The amount of the compound used may be from about 50 nM, 100 nM, 200 nM, 250 nM, 500 nM, 750 nM, 1 μM, 10 μM, 25 μM, 50 μM, 75 μM, 100 μM, 200 μM, 250 μM, 300 μM, 400 μM, 500 μM, 600 μM, 700 μM, 750 μM, 800 μM, 900 μM, 1 mM, 2 mM, 3 mM, 4 mM, 5 mM, 7.5 mM, to about 10 mM, or any range derivable therein.

In some embodiments, the charged pi-conjugated compounds of the present disclosure have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the art, whether for use in the indications stated herein or otherwise.

The charged pi-conjugated compounds of the present disclosure may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended unless the specific stereochemistry or isomeric form is specifically indicated. Compounds may occur as racemates and racemic mixtures, single enantiomers, diastereomeric mixtures, and individual diastereomers. In some embodiments, a single diastereomer is obtained. The chiral centers of the charged pi-conjugated compounds of the present disclosure can have the S or the R configuration. In some embodiments, the present compounds may contain two or more atoms that have a defined stereochemical orientation.

Chemical formulas used to represent the charged pi-conjugated compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended. The charged pi-conjugated compounds also have several resonance hybrid structures. And of the resonance structures of the molecules drawn here would be acceptable.

In addition, atoms making up the charged pi-conjugated compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, the charged pi-conjugated compounds of the present disclosure function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the disclosure contemplates prodrugs of the charged pi-conjugated compounds of the present disclosure as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, the charged pi-conjugated compounds of the present disclosure exist in salt or non-salt form. With regard to the salt form(s), in some embodiments, the particular anion or cation forming a part of any salt form of a compound provided herein is not critical so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. It is contemplated that the present methods include all different polymorphs of the compounds used herein. All solid forms of the charged pi-conjugated compounds provided herein, including any solvates thereof, are within the scope of the present invention.

### B. Formulations

In another aspect, for administration to a patient in need of such treatment, pharmaceutical formulations (also referred to as pharmaceutical preparations, pharmaceutical compositions, pharmaceutical products, medicinal products, medicines, medications, or medicaments) comprise a therapeutically effective amount of a charged pi-conjugated compounds disclosed herein formulated with one or more excipients and/or drug carriers appropriate to the indicated route of administration. In some embodiments, the charged pi-conjugated compounds disclosed herein are formulated in a manner amenable to the treatment of human and/or veterinary patients. In some embodiments, the formulation comprises admixing or combining one or more of the charged pi-conjugated compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the charged pi-conjugated compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., topically, orally, by inhalation, or by injection (e.g., subcutaneous, intravenous, and intraperitoneal). Depending on the route of administration, the charged pi-conjugated compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes or a diluent. Pharmaceutically acceptable diluents include saline and aqueous buffer solutions. Liposomes include water-in-oil-in-water emulsions as well as conventional liposomes.

The charged pi-conjugated compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as glycerol, propylene glycol, liquid polyethylene glycol, and the like), suitable mixtures thereof, and vegetable oils. The proper fluidity can be maintained, for example, by the use of a coating such as lecithin, by the maintenance of the required particle size in the case of dispersion, and by the use of surfactants.

Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols, such as mannitol and sorbitol, in the composition. Prolonged absorption of the injectable compositions can be brought about by including in the composition an agent which delays absorption, for example, aluminum monostearate or gelatin.

The charged pi-conjugated compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the charged pi-conjugated compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes, such as the inside of the mouth. Such administration can be directly to a particular location within the mucosal membrane, such as a tooth, a sore, or an ulcer. Alternatively, if local delivery to the lungs is desired, the therapeutic compound may be administered by inhalation in a dry-powder or aerosol formulation.

In some embodiments, it may be advantageous to formulate parenteral compositions in dosage unit form for ease of administration and uniformity of dosage. Dosage unit form, as used herein, refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the invention is dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

In some embodiments, active compounds are administered at a therapeutically effective dosage sufficient to treat a condition associated with a disease in a patient. For example, the efficacy of a compound can be evaluated in an animal model system that may be predictive of its efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22 (3): 659-661, 2008, which is incorporated herein by reference):

$$HED ( mg / kg ) = Animal dose ( mg / kg ) \times ( Animal {K}_{m} / Human {K}_{m} )$$

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of charged pi-conjugated compounds of the present disclosure or composition comprising a compound of the present disclosure administered to a patient may be determined by physical and physiological factors such as type of animal treated, age, sex, body weight, the severity of the condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day, with a range of 750 mg to 9,000 mg per day.

In some embodiments, the amount of the active compound in the pharmaceutical formulation is from about 2 to about 75 weight percent. In some of these embodiments, the amount is from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein, a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, on a weekly basis, a monthly basis, or any set number of days or weeks there-between. Alternatively, the predetermined routine schedule may involve administration on a twice-daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

### II. INDICATIONS

### A. Infections

In some embodiments, the cell-targeting moiety may target a bacterial cell, a protozoan cell, a virus, a fungal cell, or another type of parasitic cell. In this disclosure, the charged pi-conjugated compounds described herein may be used to reduce the number of pathogen cells and, as such, can potentially be used to treat a variety of diseases or conditions associated with or caused by bacteria, protozoa, viruses, fungi, or other types of parasitic cells. In some embodiments, the charged pi-conjugated compounds described herein are contemplated to permeabilize the cell membrane to allow at least a second therapeutic agent to enter a bacterial cell, a protozoan cell, a virus, a fungal cell, or another type of parasitic cell. In some aspects, it is anticipated that the charged pi-conjugated compounds described herein may be used to treat virtually any malignancy associated with or caused by bacteria, protozoa, viruses, fungi, or other types of parasitic cells.

i. Bacterial Pathogens

There are hundreds of bacterial pathogens from both the Gram-positive and Gram-negative families that cause significant illness and mortality around the world, despite decades of efforts to develop antibiotics. Indeed, antibiotic resistance is a growing challenge in combating bacterial diseases.

One of the bacterial diseases with the highest disease burden is tuberculosis, caused by the bacterium Mycobacterium tuberculosis, which kills about 2 million people a year, mostly in sub-Saharan Africa. Some non-limiting examples of Mycobacterium tuberculosis antigens include recombinant Ag85A, Ag85B, ESAT6, TB10.4, or fragments thereof, including those taught by Ottenhoff and Kaufmann, 2012, which are incorporated herein by reference. Pathogenic bacteria contribute to other globally important diseases, such as pneumonia, which can be caused by bacteria such as Streptococcus, Staphylococcus, Klebsiella, Pseudomonas, and Escherichia, and foodborne illnesses, which can be caused by bacteria such as Shigella, Campylobacter, Salmonella, Listeria, Clostridium, and Vibrio. Pathogenic bacteria also cause infections such as tetanus, typhoid fever, diphtheria, syphilis, and leprosy, as well as urinary tract infections, skin infections, and other types of illnesses.

Conditionally pathogenic bacteria are only pathogenic under certain conditions, such as when a wound facilitates the entry of bacteria into the bloodstream or when the immune system is weakened. For example, Staphylococcus and Streptococcus are part of the normal human flora and usually exist on the skin or in the nose without causing disease, but can potentially cause skin infections, pneumonia, meningitis, and even overwhelming sepsis, a systemic inflammatory response producing shock, massive vasodilation, and death. Some species of bacteria, such as Pseudomonas aeruginosa, Burkholderia cenocepacia, and Mycobacterium avium, are opportunistic pathogens and cause disease, mainly in people suffering from immunosuppression or cystic fibrosis.

Other bacteria invariably cause disease in humans, such as obligate intracellular parasites (e.g., Chlamydia, Ehrlichia, Rickettsia), which can only grow and reproduce inside the cells of other organisms and can cause a variety of illnesses, from asymptomatic infections during the incubation period to more serious diseases including meningitis, sepsis, and abscesses. Rickettsia, for example, can cause typhus or Rocky Mountain spotted fever, while Chlamydia can lead to pneumonia, genital infections, eye infections, and coronary heart disease. Other facultative (not obligate) intracellular parasites include Mycobacterium, which can cause tuberculosis and leprosy; Brucella, which can cause brucellosis; Francisella, which can cause tularemia; Legionella, which can cause Legionnaires' disease; and Listeria, which can cause listeriosis; Bordetella pertussis, which can cause whooping cough; Yersinia pestis, which can cause plague; and Borrelia burgdorferi, which can cause Lyme disease. Cell membranes for these bacteria may be disrupted using the methods described herein.

ii. Viral Pathogens

Viral pathogens are important health concerns. These pathogens include respiratory viruses such as adenoviruses, influenza A and B viruses, measles, parainfluenza virus, respiratory syncytial virus (RSV), rhinoviruses, SARS-COV, MERS-COV, and SARS-COV-2, gastroenteric viruses such as Coxsackie viruses, enteroviruses such as poliovirus and rotavirus, hepatitis viruses such as hepatitis B virus (HBV), hepatitis C virus, and bovine viral diarrhea virus (BVDV), herpesviruses such as human papillomavirus (HPV), herpes simplex virus 1 (HSV-1) and herpes simplex virus 2 (HSV-2), human cytomegalovirus (HCMV), and varicella-zoster virus (VZV), retroviruses such as human immunodeficiency virus type 1 (HIV-1) and human immunodeficiency virus type 2 (HIV-2), as well as dengue virus, hantavirus, hemorrhagic fever viruses, lymphocytic choriomeningitis virus, smallpox virus, norovirus, chikungunya virus, Ebola virus, rabies virus, West Nile virus envelope protein (E) and pre-membrane protein (prM), dengue virus E protein, HIV envelope proteins gp41 and gp120, rotavirus VP6 and enterovirus VP1, human papillomavirus type 1 (HPV-1), or fragments thereof. Some non-limiting viral antigens include hepatitis B virus HBV surface and core antigens, influenza virus haemagglutinin and neuraminidase antigens, West Nile virus envelop protein (E) and pre-membrane protein (prM), dengue virus E protein, Ebola virus glycoprotein, HIV envelope proteins gp41 and gp120, rotavirus VP6 and enterovirus VP1, human papillomavirus type 1 (HPV-1), or fragments thereof. Other HIV antigens can be found in de Taeye, et al., 2016, which is incorporated herein by reference. The cell membranes for any of these viral pathogens may be disrupted using the methods described herein.

iii. Fungal Pathogens

Pathogenic fungi are fungi that cause disease in humans or other organisms. The following are but a few examples.

Candida species are important human pathogens that are best known for causing opportunist infections in immunocompromised hosts, such as transplant patients, people living with HIV and AIDS, and cancer patients. These infections can be difficult to treat and can be very serious. Aspergillus species can cause disease through the production of mycotoxins, induction of allergenic responses, and localized or systemic infections. The type of infection depends on the immune status of the host, and the most common pathogenic species are Aspergillus fumigatus and Aspergillus flavus, but other species can also cause disease. Cryptococcus neoformans is the most common species that can cause a severe form of meningitis and meningo-encephalitis in patients with HIV infection and AIDS. The majority of Cryptococcus species live in the soil and do not cause disease in humans; however, Cryptococcus laurentii and Cryptococcus albidus have been known to occasionally cause moderate-to-severe disease in human patients with compromised immunity. Cryptococcus gattii is endemic to tropical parts of the continent of Africa and Australia and can cause disease in both immunocompromised and immunocompetent people. Histoplasma capsulatum can cause histoplasmosis in humans, dogs, cats, birds, and reptiles, particularly in areas with high levels of bird or bat droppings. Pneumocystis jirovecii (or Pneumocystis carinii) can cause a form of pneumonia in people with weakened immune systems, such as premature children, the elderly, transplant patients, and AIDS patients, as well as in immunocompetent people. Stachybotrys chartarum, or “black mold,” can cause respiratory damage and severe headaches, and it frequently occurs in houses in regions that are chronically damp. Additionally, Coccidioides immitis can cause a severe infection known as Valley Fever which is most prevalent in dry and semiarid regions in North and South America, Paracoccidioides brasiliensis can cause paracoccidioidomycosis, which is the most common systemic mycosis in Latin America, Trichophyton rubrum can cause dermatophytosis, a type of fungal skin infection, and Alternaria alternata can cause allergic reactions such as asthma, hay fever, and rhinitis. Cell membranes from these fungi may be disrupted using the methods described herein.

iv. Parasites

Parasites present a major health issue, particularly in under-developed countries around the world. Significant pathogenic parasites include Entamoeba histolytica, Giardia lamblia, Trichomonas vaginalis, Plasmodium falciparum, Plasmodium malariae, Plasmodium ovale, Plasmodium vivax, Trypanosoma gambiense, Trypanosoma rhodesiense, Trypanosoma cruzi, Ascaris lumbricoides, Trichinella spiralis, Toxoplasma gondii, Leishmania donovani, Leishmania tropica, Leishmania braziliensis, Schistosoma mansoni, Schistosoma japonicum, Schistosoma haematobium, Pneumocystis jiroveci, Wuchereria bancrofti, Ancylostoma duodenale, Necator americanus, Strongyloides stercoralis, Cryptosporidium parvum, Enterobius vermicularis, and Taenia solium. Cell membranes from these parasites may be disrupted using the methods described herein.

v. Cancer and Other Hyperproliferative Diseases and Disorders

While hyperproliferative diseases can be associated with any disease which causes a cell to begin to reproduce uncontrollably, cancer is the common example. One of the key elements of cancer is that the cell's normal apoptotic cycle is interrupted and thus agents that lead to apoptosis of the cell are important therapeutic agents for treating these diseases. In this disclosure, the compounds of the present disclosure have been shown to kill cancer cells and as such can potentially be used to treat a variety of types of cancer lines. In some embodiments, the present methods involve killing a cancer cell via a programmed cell death mechanism, such as apoptosis. In some embodiments, the present methods represent an improvement over known methods in that photoactivation is not required. In some embodiments, the present methods represent an improvement over known methods in that photothermal activation is not required. In some embodiments, the present methods represent an improvement over known methods in that the methods do not involve generation of reactive oxygen species.

In some embodiments, the methods involve selective targeting of cancer cells by charged pi-conjugated compounds. As such, the compounds of the present disclosure may be used to effectively treat cancers such as melanoma. In various aspects, it is anticipated that compounds of the present disclosure may be used to treat virtually any malignancy.

Cancer cells that may be treated with the compounds of the present disclosure according to the embodiments include but are not limited to cells from the bladder, blood, bone, bone marrow, brain, breast, colon, esophagus, gastrointestine, gum, head, kidney, liver, lung, nasopharynx, neck, oral, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### III. CELL TARGETING MOIETIES

In some aspects, the present disclosure provides compounds conjugated directly or through linkers to a cell-targeting moiety. In some embodiments, the conjugation of the compound to a cell-targeting moiety enables the charged pi-conjugated compounds to bind to and penetrate the target cell more effectively, thereby increasing the specificity and efficacy of the compound in treating a disease or disorder, while avoiding non-target cells and reducing the toxicity of the antimicrobial therapy. The target cell can be a microbial pathogen directly or a mammalian cell infected with an intracellular pathogen.

Cell targeting moieties according to the embodiments may be, for example, an antibody, a lipid, a carbohydrate, a polysaccharide, a growth factor, a peptide, an aptamer, a small molecule such as a hormone, an imaging agent, or cofactor, or a cytokine. In some embodiments, the cell-targeting moiety is a functional group that associates with the cell membrane, a carbohydrate or polysaccharide that binds to one or more markers on the cell membrane, a lipid that binds to one or more markers on the cell membrane, a small molecule that binds to one or more markers on the cell membrane, an aptamer that binds to one or more markers on the cell membrane, or a peptide or an antibody that binds to one or more markers on the cell membrane. In some embodiments, the cell-targeting moiety may target a human cell infected by an intracellular pathogen, whereby the cell-targeting moiety recognizes specific markers or molecules that are produced by the pathogen and present on the infected cell surface while avoiding those found on healthy cells. These specific markers include adhesion molecules, cell-surface receptors, surface enzymes, lipopolysaccharides, lipoproteins, peptidoglycans, and other pathogen-specific proteins and/or metabolites. Thus, in some embodiments, the compounds of the present disclosure may be used in conjugates with, for instance, an antibody for a specific antigen that is expressed by an infected cell but not a healthy cell. The conjugates can be formed by covalent, electrostatic, or hydrophobic interactions between the cell-targeting moiety and the antibody, which allows the antibody to target and bind to the infected cell specifically.

In certain embodiments, the cell-targeting group is a functional group, such as a positively charged group like an amine. The positively charged group may be used to associate with the negatively charged groups at the surface of the cell membrane. It is contemplated that this group might be used to associate with other negatively charged groups, such as negatively charged phospholipids, proteins, or nucleic acids.

Additionally, specific cell surface molecules that are overproduced by infected mammalian cells can be used as cell-targeting moieties for antimicrobial therapy with the charged pi-conjugated compounds. These include molecules such as lectins, cell adhesion molecules, and integrins that are overexpressed on the surface of infected cells and can be used as cell-specific targeting moieties in antimicrobial therapy. Some cell surface receptors are also overexpressed by infected mammalian cells, including Toll-like receptors, C-type lectins, and mannose receptors, which can be targeted to deliver the charged pi-conjugated compounds specifically to the infected cells.

Additionally, the cell-targeting moieties that may be used include a cofactor, a sugar, a drug molecule, an imaging agent, or a fluorescent dye. Other molecules, such as cytokines, chemokines, and transcription factors, can also be used as cell-targeting moieties for certain types of infections by binding to specific receptors on the surface of infected cells. For example, cytokines are proteins that are released by cells in response to infection and can bind to receptors on infected cells, triggering a variety of responses such as increased inflammation, increased production of infection-fighting molecules, or increased movement of immune cells to the site of infection. Specific cytokines that can be used as cell-targeting moieties for antibacterial therapy include tumor necrosis factor (TNF), interleukin-1 (IL-1), and interferon-γ (IFN-γ). Chemokines are molecules that are released by cells to attract and recruit other cells to the site of infection. Specific chemokines that can be used as cell-targeting moieties include interleukin-8 (IL-8), monocyte chemoattractant protein-1 (MCP-1), and macrophage inflammatory protein-la (MIP-1α). Transcription factors are molecules that control the expression of certain genes in cells and can be used to target specific genes that are essential for the replication of the pathogen. By binding to these receptors, the cell-targeting moieties can direct the charged pi-conjugated compounds specifically to the infected cells while avoiding uninfected cells. Specific transcription factors that can be used as cell-targeting moieties include NF-κB and AP-1.

Furthermore, in some aspects, the cell-targeting moiety may be a peptide sequence or a cyclic peptide designed to recognize and bind to a specific receptor or molecule on the infected cell surface, thus allowing the charged pi-conjugated compounds to be delivered directly to the infected cells while avoiding uninfected cells. Examples of cell- and tissue-targeting peptides that may be used according to the embodiments include peptides that recognize bacterial flagellin proteins, peptidoglycan, lipopolysaccharide, or lipoprotein molecules. Examples of such targeting peptides are provided, for instance, in U.S. Pat. Nos. 9,072,793, 9,597,407, and U.S. Patent Application No. 2017/0362307, each of which is incorporated herein by reference.

Thus, in some embodiments, cell-targeting moieties are antibodies or avimers. Antibodies and avimers can be generated against virtually any cell surface marker, thus providing a method for targeted delivery of the charged pi-conjugated compounds to virtually any cell population of interest. Methods for generating antibodies that may be used as cell-targeting moieties are detailed below. Methods for generating avimers that bind to a given cell surface marker are detailed in U.S. Patent Publications Nos. 2006/0234299 and 2006/0223114, each incorporated herein by reference.

Additionally, it is contemplated that the compounds described herein may be conjugated to a nanoparticle or other nanomaterial. Some non-limiting examples of nanoparticles include metal nanoparticles such as gold or silver nanoparticles or polymeric nanoparticles such as poly-L-lactic acid or poly(ethylene) glycol polymers. Nanoparticles and nanomaterials which may be conjugated to the instant compounds include those described in U.S. Patent Publications Nos. 2006/0034925, 2006/0115537, 2007/0148095, 2012/0141550, 2013/0138032, and 2014/0024610 and PCT Publication No. 2008/121949, 2011/053435, and 2014/087413, each incorporated herein by reference.

### IV. THERAPIES

### A. Methods of Treatment

In particular, the compositions that may be used in treating a disease or disorder in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., slowing, stopping, reducing or eliminating one or more symptoms or underlying causes of disease). The toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms and other drugs being administered concurrently. In some embodiments, the amount of the charged pi-conjugated compounds used is calculated to be from about 0.01 mg to about 10,000 mg/day. In some embodiments, the amount is from about 1 mg to about 1,000 mg/day. In some embodiments, these dosings may be reduced or increased based on the biological factors of a particular patient, such as increased or decreased metabolic breakdown of the drug or decreased uptake by the digestive tract if administered orally. Additionally, the charged pi-conjugated compounds may be more efficacious, and thus a smaller dose is required to achieve a similar effect. Such a dose is typically administered once a day for a few weeks or until sufficient to achieve clinical benefit.

The therapeutic methods of the disclosure (which include prophylactic treatment) in general include the administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., microbiological analysis, genetic test, enzyme or protein marker, family history, and the like).

### B. Combination Therapies

It is envisioned that the charged pi-conjugated compounds described herein may be used in combination therapies with one or more additional therapies or a compound that mitigates one or more of the side effects experienced by the patient. It is common in the field of medicine to combine therapeutic modalities. The following is a general discussion of therapies that may be used in conjunction with the therapies of the present disclosure.

To treat diseases or disorders using the methods and compositions of the present disclosure, one would generally contact a cell or a subject with a charged pi-conjugated compounds and at least one other therapy. These therapies would be provided in a combined amount effective to achieve a reduction in one or more disease parameters. This process may involve contacting the cells/subjects with both agents/therapies at the same time, e.g., using a single composition or pharmacological formulation that includes both agents, or by contacting the cell/subject with two distinct compositions or formulations, at the same time, wherein one composition includes the compound and the other includes the other agent.

Alternatively, the compounds described herein may precede or follow the other treatment by intervals ranging from minutes to weeks. One would generally ensure that a significant period of time did not expire between the times of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 1-2 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the compound or the other therapy will be desired. Various combinations may be employed, where a compound of the present disclosure is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are also contemplated. A discussion of other potential therapies that may be used in combination with the compounds of the present disclosure is presented elsewhere in this document.

### V. CHEMISTRY BACKGROUND

In some aspects, the charged pi-conjugated compounds of this disclosure can be synthesized using the methods of organic chemistry as described in this application. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

### A. Process Scale-Up

The synthetic methods described herein can be further modified and optimized for preparative, pilot- or large-scale production, either batch or continuous, using the principles and techniques of process chemistry as applied by a person skilled in the art. Such principles and techniques are taught, for example, in Practical Process Research & Development (2000), which is incorporated by reference herein. The synthetic method described herein may be used to produce preparative scale amounts of the compounds described herein.

### B. Chemical Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

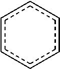

covers, for example,

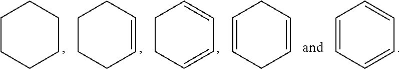

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

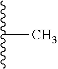

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page.” The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

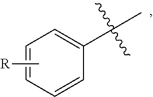

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

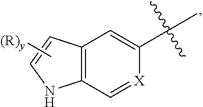

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C═n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C1-4-alkyl”, “alkyl_{(C1-4)}”, and “alkyl_{(C≤4)}” are all synonymous. Except as noted below, every carbon atom is counted to determine whether the group or compound falls within the specified number of carbon atoms. For example, the group dihexylamino is an example of a dialkylamino_{(C12)} group; however, it is not an example of a dialkylamino_{(C6)} group. Likewise, phenylethyl is an example of an aralkyl_{(C=8)} group. When any of the chemical groups or compound classes defined herein is modified by the term “substituted,” any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon-oxygen double bond or a carbon-nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

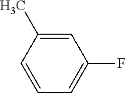

is also taken to refer to

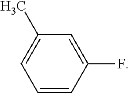

Aromatic compounds may also be depicted using a circle to represent the delocalized nature of the electrons in the fully conjugated cyclic π system, two non-limiting examples of which are shown below:

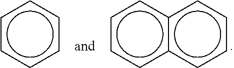

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃) CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

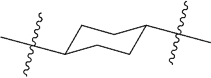 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl.

The term “aryl” refers to a monovalent unsaturated aromatic group with an aromatic carbon atom as the point of attachment, said carbon atom forming part of one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

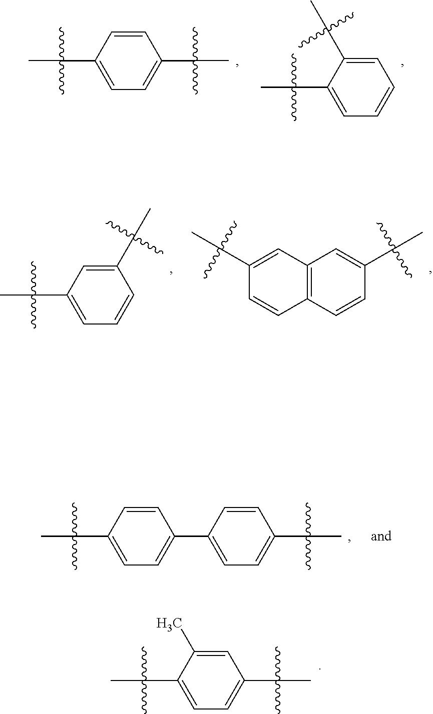

An “arene” refers to the class of compounds having the formula H—R, wherein R is aryl as that term is defined above. Benzene and toluene are non-limiting examples of arenes.

The term “aralkyl” refers to the monovalent group-alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

The term “heteroaralkyl” refers to the monovalent group-alkanediyl-heteroaryl, in which the terms alkanediyl and heteroaryl are each used in a manner consistent with the definitions provided above. Non-limiting examples are: pyridinylmethyl and 2-quinolinyl-ethyl.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group.

The term “heterocycloalkalkyl” refers to the monovalent group -alkanediyl-heterocycloalkyl, in which the terms alkanediyl and heterocycloalkyl are each used in a manner consistent with the definitions provided above. Non-limiting examples are: morpholinylmethyl and piperidinylethyl.

The term “acyl” refers to the group-C(O)R, in which R is a hydrogen, alkyl, cycloalkyl, or aryl as those terms are defined above. The groups, CHO, —C(O)CH₃ (acetyl, Ac), —C(O)CH₂CH₃, —C(O)CH(CH₃)₂, —C(O)CH(CH₂)₂, C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group-OR, in which R is an alkyl, as that term is defined above. Non-limiting examples include: —OCH₃ (methoxy), 13 OCH₂CH₃ (ethoxy), —OCH₂CH₂CH₃, —OCH(CH₃)₂ (isopropoxy), or —OC(CH₃)₃ (tert-butoxy). The terms “cycloalkoxy,” “alkenyloxy,” “alkynyloxy,” “aryloxy,” “aralkoxy,” “heteroaryloxy,” “heterocycloalkoxy,” and “acyloxy,” when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, C(O)NH₂, C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups:—CH₂OH, —CH₂Cl, —CF₃, —CH₂CN, —CH₂C(O)OH, —CH₂C(O) OCH₃, —CH₂C(O)NH₂, —CH₂C(O) CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O) OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients. Unless otherwise noted, the term “about” is used to indicate a value of +10% of the reported value, preferably a value of +5% of the reported value. It is to be understood that, whenever the term “about” is used, a specific reference to the exact numerical value indicated is also included.”

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective” or “sufficient”, as such terms are used in the specification and/or claims, means adequate to accomplish a desired, expected, or intended result. “Effective amount,” “therapeutically effective amount,” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of anti-adherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e., an enzyme, cell, cell receptor or microorganism) by half. The term “EC₅₀” refers to an amount that is an effective concentration that results in a half-maximal response.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants, and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases.

Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient of the present invention. The prodrug itself may or may not have activity in its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus, or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

As used herein, the term “catalyst” refers to any compound or composition which facilitates progression of a reaction (i.e., increases the rate of a reaction) without being consumed by the reaction. A catalyst of the present disclosure may facilitate increased yield of a reaction at favorable reaction conditions over corresponding methods known in the art. In some embodiments, a catalyst used in presently disclosed methods is a homogeneous catalyst. In some embodiments, a catalyst used in presently disclosed methods is a heterogeneous catalyst. Particular catalysts for use in the presently disclosed methods are widely known in the art and commercially available. For example, catalysts for use in the presently disclosed methods may be found by the artisan in March's Advanced Organic Chemistry: Reactions, Mechanisms, and Structure. A non-limiting example of a catalyst that may be used in the presently disclosed methods is tetrakis(triphenylphosphine) palladium, also referenced herein as Pd(PPh₃).

As used herein, the term “base” refers to any compound or composition which, in a reaction with another compound or composition, acts as an Arrenhius base, a Bronsted base, or a Lewis base, as understood by a person of skill in the art. In some embodiments, a base according to the present disclosure reacts with another compound or composition to accept or remove a proton (H⁺) from the other compound or composition. In some embodiments, the base is an organic base. In some embodiments, the base is an inorganic base. A base used according to the present disclosure may be selected due to the pKa of the base, or for any other property possessed by the base that is deemed favorable by the artisan. Particular bases for use in the presently disclosed methods are widely known in the art and may be found by the artisan in texts or literature, such as March's Advanced Organic Chemistry: Reactions, Mechanisms, and Structure. Nonlimiting examples of bases that may be used in the presently disclosed methods include K₃PO₄, K₂HPO₄, KF, K₂CO₃, NaOtBu, NaOAc, CsF, or Cs₂CO₃.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### VI. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Synthesis of Charged Pi-Conjugated Compounds

i. Synthesis of Charged Pi-Conjugated Compounds and Characterization of Charged Pi-Conjugated Compounds

The charged pi-conjugated compounds were synthesized and then characterized with NMR spectroscopy, High Resolution Mass Spectroscopy, and Fourier-transform infrared spectroscopy. The chemical shift information and observed mass data is given within the synthetic description.

General information: All glassware was oven-dried overnight prior to use. All reactions were carried out under an N₂ atmosphere unless otherwise noted. All other chemicals were purchased from commercial suppliers and used without further purification.

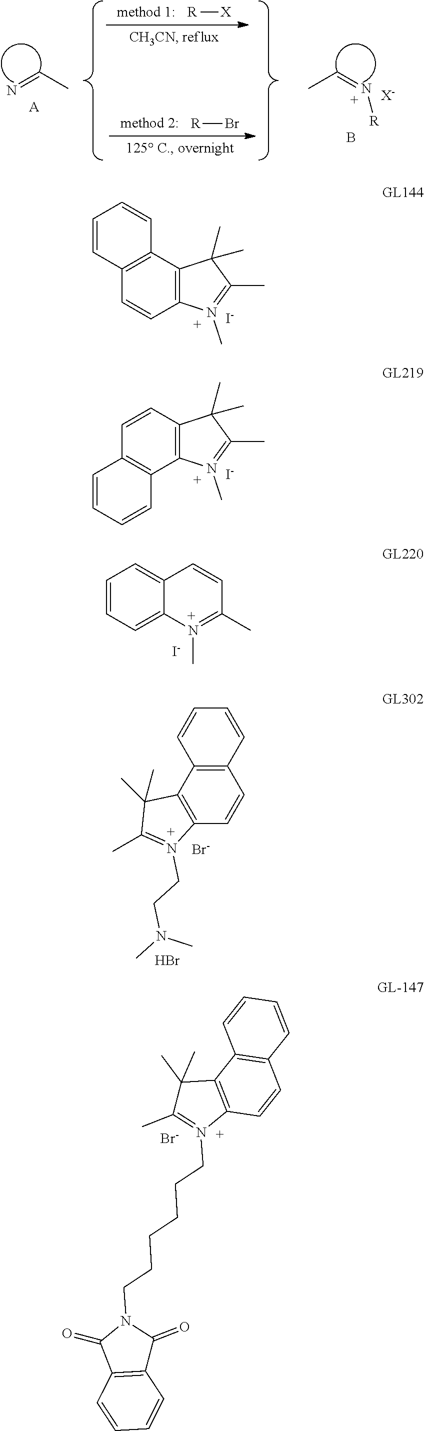

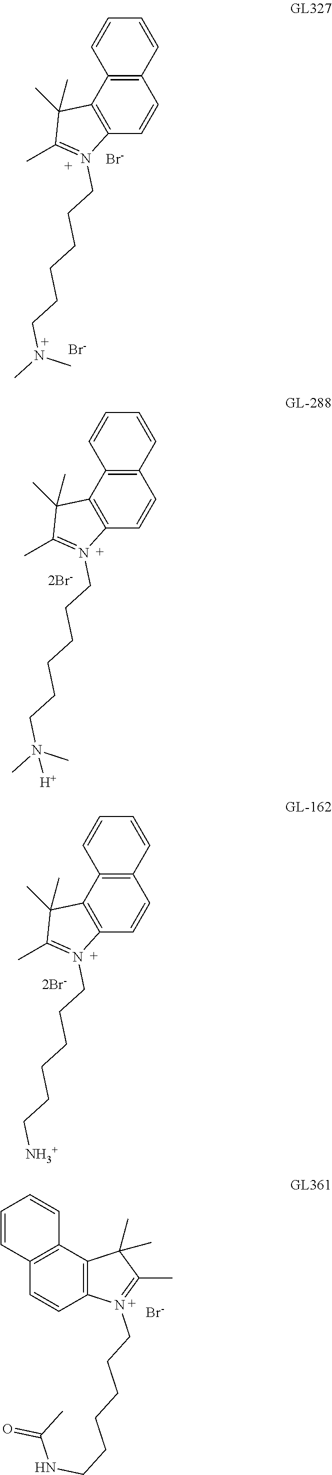

### General Procedure for the Synthesis of Heterocyclic Salt:

Method 1: Compound A (1 equiv.) and R—X (X=halogen or other leaving group such as OTs, OMs, OTf, and so throughout) (1.2-1.5 equiv.) were mixed in CH₃CN in a screwed-capped vial. The mixture was stirred and heated to reflux overnight. The solid was filtered and washed with ether and dried in vacuum.

Method 2: Compound A (1 equiv.) and R—Br (1.0 equiv.) were mixed in an 8 mL screwed-capped vial. The mixture was stirred and heated to 125° C. overnight. The blank solid was washed with ethyl acetate ether and recrystallized with methanol/ether.

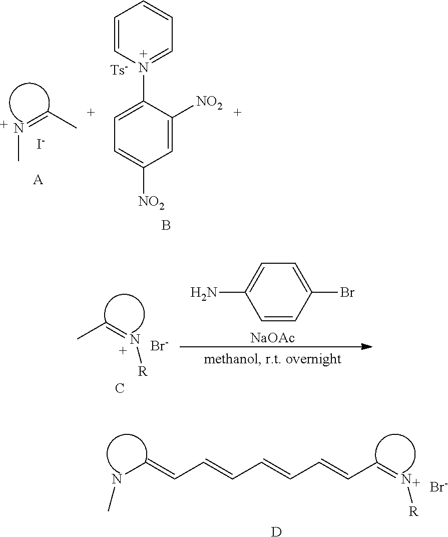

General procedure: The corresponding pyridinium salt B (1 equiv.) and 4-bromoaniline (1 equiv.) were dissolved in methanol (4 mL) in an 8 mL vial, and the mixture was stirred at room temperature for 30 min. Next, a heterocyclic salt A (1 equiv.), C (1 equiv.) and sodium acetate (3 equiv.) were added. The reaction mixture was stirred for an additional overnight at room temperature. The crude product D was purified by flash column chromatography (silica gel, dichloromethane/methanol).

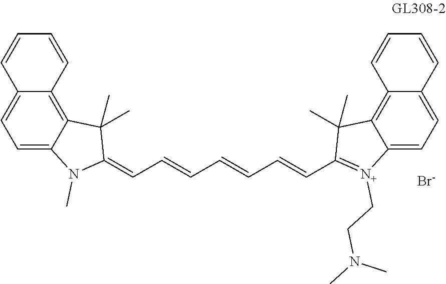 [SMILES: CN(C)CC[N+]1=C(/C=C/C=C/C=C/C=C2/N(C)c3ccc4ccccc4c3C2(C)C)C(C)(C)c2c1ccc1ccccc21 ; [Br-]]

The corresponding pyridinium salt 175 (206 mg, 0.48 mmol) and 4-bromoaniline (103 mg, 0.3 mmol) were dissolved in methanol (4 mL, 7 mL/mmol), and the mixture was stirred at room temperature for 30 min. Next, a heterocyclic salt 144 (176 mg, 0.5 mmol), 302 (286 mg, 0.65 mmol) and sodium acetate (246 mg) were added, and the reaction mixture was stirred for an additional 16 h at room temperature. The crude product was purified by flash column chromatography (silica gel, dichloromethane/methanol, 20:1, then 10:1), affording 58 mg of green solid GL308-2. Yield: 19%, 1H NMR (600 μMHz, Methanol-d4) δ 8.27 (d, J=8.17 Hz, 1H), 8.23 (d, J=8.17 Hz, 1H), 8.11 (t, 1H), 8.06-7.98 (m, 5H), 7.69-7.63 (m, 4H), 7.55 (d, J=8.80 Hz, 1H), 7.52 (t, 1H), 7.48 (t, 1H), 6.67-6.58 (m, 2H), 6.42 (d, J=13.82 Hz, 1H), 6.27 (d, J=13.82 Hz, 1H), 4.29 (t, 2H), 3.79 (s, 3H), 2.77 (t, 2H), 2.43 (s, 6H), 2.02 (d, J=4.66 Hz, 12H). ¹³C NMR (150 μMHz, Methanol-d4) δ 174.95, 171.89, 171.50, 149.47, 140.24, 139.83, 133.86, 132.70, 132.20, 131.76, 130.37, 130.24, 129.74, 129.69, 128.20, 127.95, 127.41, 127.24, 125.96, 125.61, 124.85, 124.32, 122.01, 121.84, 110.66, 110.25, 104.22, 102.16, 55.27, 51.09, 50.45, 48.16, 44.52, 41.59, 30.71, 26.27, 25.97. HRMS (ESI) for C₄₀H₄₄N₃⁺ [M-Br]⁺: 566.3530. Found: 566.3534.

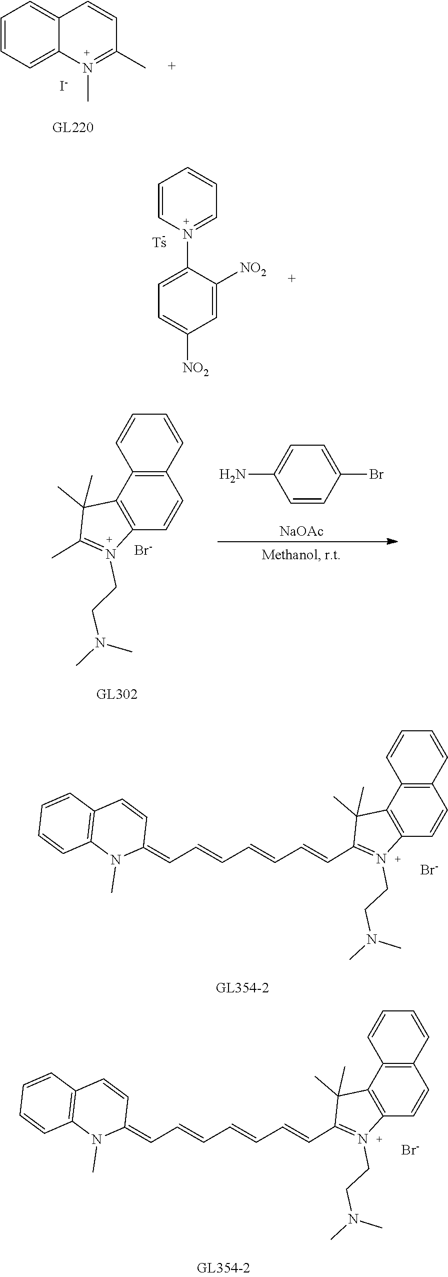

The corresponding pyridinium salt GL175 (209 mg, 0.5 mmol) and 4-bromoaniline (103 mg, 0.65 mmol) were dissolved in pyridine (4 mL, 7 mL/mmol), and the mixture was stirred at room temperature for 30 min. Next, a heterocyclic salt GL220 (143 mg, 0.5 mmol), GL302 (286 mg, 0.65 mmol) and sodium acetate (246 mg) were added, and the reaction mixture was stirred for an additional 16 h at room temperature. The crude product was purified by flash column chromatography (silica gel, dichloromethane/methanol, 20:1, 10:1). 80 mg of green solid was obtained. Yield: 27.5%, 1H NMR (600 μMHz, Methanol-d4) δ 8.48 (d, J=9.20 Hz, 1H), 8.23 (s, J=9.15 Hz, 2H), 8.13-8.00 (m, 4H), 7.87 (d, J=8.52 Hz, 2H), 7.75 (t, 1H), 7.62 (t, 1H), 7.52 (t, 1H), 7.33 (m, 3H), 6.96 (d, J=13.83 Hz, 1H), 6.69 (t, 1H), 6.42 (t, 1H), 5.84 (d, J=12.51 Hz, 1H), 4.31 (s, 3H), 4.08 (t, 2H), 2.70 (t, 2H), 2.45 (s, 6H), 1.95 (s, 6H), ¹³C NMR (150 μMHz, Methanol-d4) δ 164.55, 155.60, 150.45, 141.82, 140.81, 140.41, 139.80, 133.82, 130.69, 129.88, 129.72, 129.58, 129.55, 128.69, 127.33, 126.82, 126.70, 126.28, 124.68, 122.86, 121.44, 119.68, 117.50, 114.24, 109.31, 98.05, 54.70, 48.70, 48.05, 47.88, 44.45, 40.41, 37.20, 26.46. HRMS (ESI) for C₃₅H₃₈N₃⁺ [M-Br]⁺: 500.3060. Found: 500.3087.

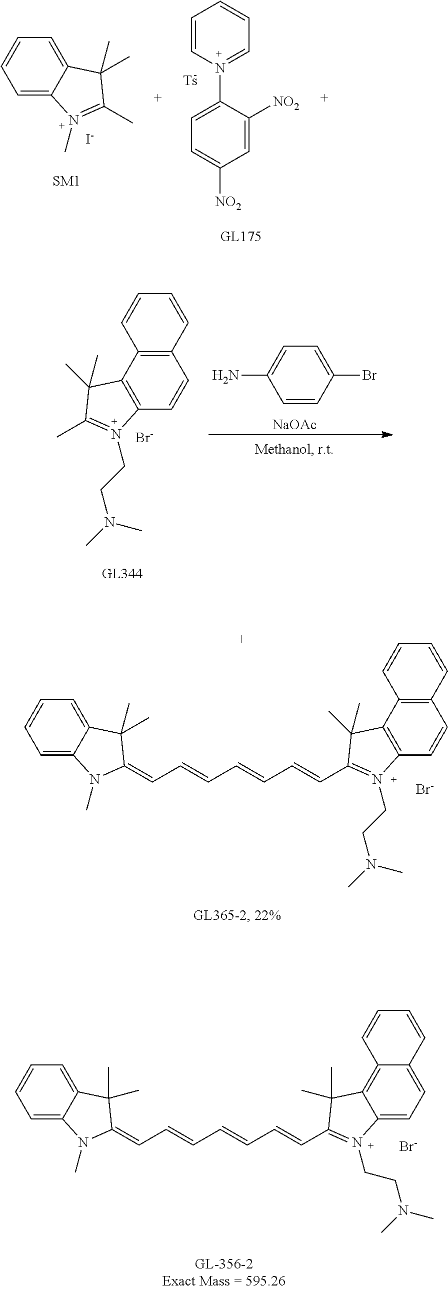

The corresponding pyridinium salt GL175 (237 mg, 0.5 mmol) and 4-bromoaniline (103 mg, 0.6 mmol) were dissolved in methanol (4 mL, 7 mL/mmol), and the mixture was stirred at room temperature for 30 min. Next, a heterocyclic salt GL176-1 (150 mg, 0.5 mmol), GL302 (286 mg, 0.65 mmol) and sodium acetate (246 mg) were added, and the reaction mixture was stirred for an additional 16 h at room temperature. The crude product was purified by flash column chromatography (silica gel, dichloromethane/methanol, 20:1, 10:1), affording 54 mg of green solid in 18.1% yield. 1H NMR (600 μMHz, Methanol-d4) δ 8.24 (d, J=8.43 Hz, 1H), 8.04-7.99 (m, 4H), 7.66-7.63 (m, 2H), 7.58 (d, J=8.95 Hz, 1H), 7.51 (m, 2H), 7.43 (m, 1H), 7.32-7.27 (m, 2H), 6.61 (t, 2H), 6.33 (m, 2H), 4.33 (t, 2H), 3.64 (s, 3H), 2.80 (t, 2H), 2.45 (s, 6H), 1.99 (s, 6H), 1.73 (s, 6H). ¹³C NMR (150 μMHz, Methanol-d4) δ 142.95, 141.05, 139.67, 133.13, 131.97, 131.31, 130.31, 129.70, 128.94, 128.67, 128.36, 128.13, 127.31, 125.79, 124.79, 124.64, 124.53, 121.89, 121.26, 110.48, 110.40, 107.96, 103.83, 102.86, 70.74, 55.28, 50.70, 48.98, 44.48, 41.71, 30.20, 26.51, 26.25. HRMS (ESI) for C₃₆H₄₂N₃⁺ [M-Br]⁺: 516.3373. Found: 516.3380.

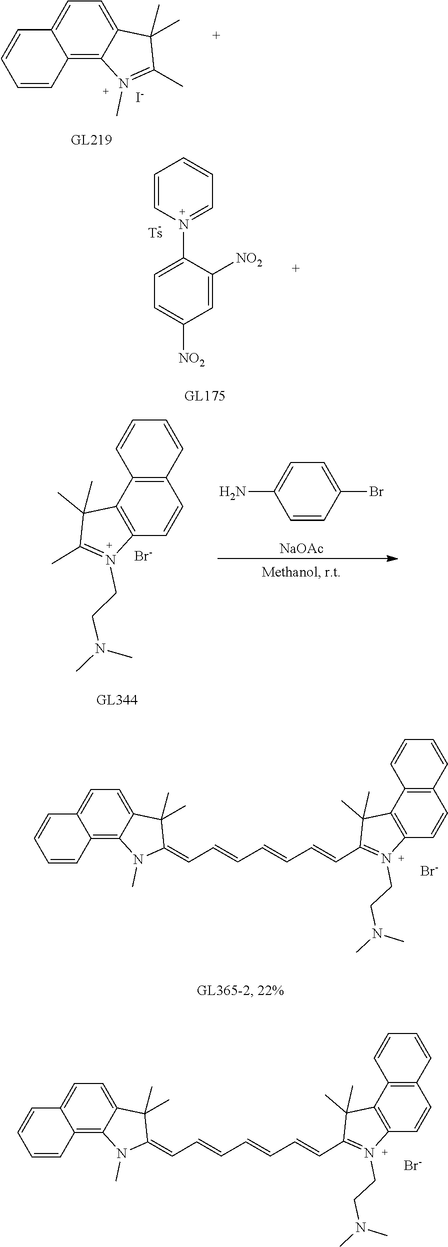

The corresponding pyridinium salt GL175 (103 mg, 0.24 mmol) and 4-bromoaniline (49 mg, 0.29 mmol) were dissolved in methanol (4 mL), and the mixture was stirred at room temperature for 30 min. Next, a heterocyclic salt GL219 (105 mg, 0.3 mmol), GL344 (132 mg, 0.3 mmol) and sodium acetate (116 mg) were added, and the reaction mixture was stirred for an additional 16 h at room temperature. The crude product was purified by flash column chromatography (silica gel, dichloromethane/methanol, 20:1, then 10:1), affording GL365-2 as a green solid in 22% yield. ¹H NMR (600 μMHz, Methanol-d4) δ 8.62 (d, J=8.83 Hz, 1H), 8.23 (d, J=8.83 Hz, 1H), 8.08-7.98 (m, 4H), 7.88-7.82 (m, 2H), 7.71-7.63 (m, 3H), 7.58 (m, 2H), 7.48 (t, 1H), 6.69-6.60 (m, 2H), 6.51 (d, J=13.55 Hz, 1H), 6.31 (d, J=13.55 Hz, 1H), 4.34 (t, 2H), 4.22 (s, 3H), 2.84 (t, 2H), 2.49 (s, 6H), 2.01 (s, 6H), 1.78 (s, 6H). ¹³C NMR (150 μMHz, Methanol-d4) δ 174.57, 139.71, 139.02, 137.44, 135.06, 131.85, 130.30, 129.70, 129.40, 128.17, 127.29, 126.73, 126.57, 125.71, 124.44, 121.87, 121.64, 121.30, 118.98, 110.30, 55.13, 53.40, 50.58, 49.00, 44.39, 43.06, 41.49, 36.15, 28.93, 26.38, 26.24, 19.84. HRMS (ESI) for C₃₅H₃₈N₃⁺ [M-Br]⁺: 500.3060. Found: 500.3087.

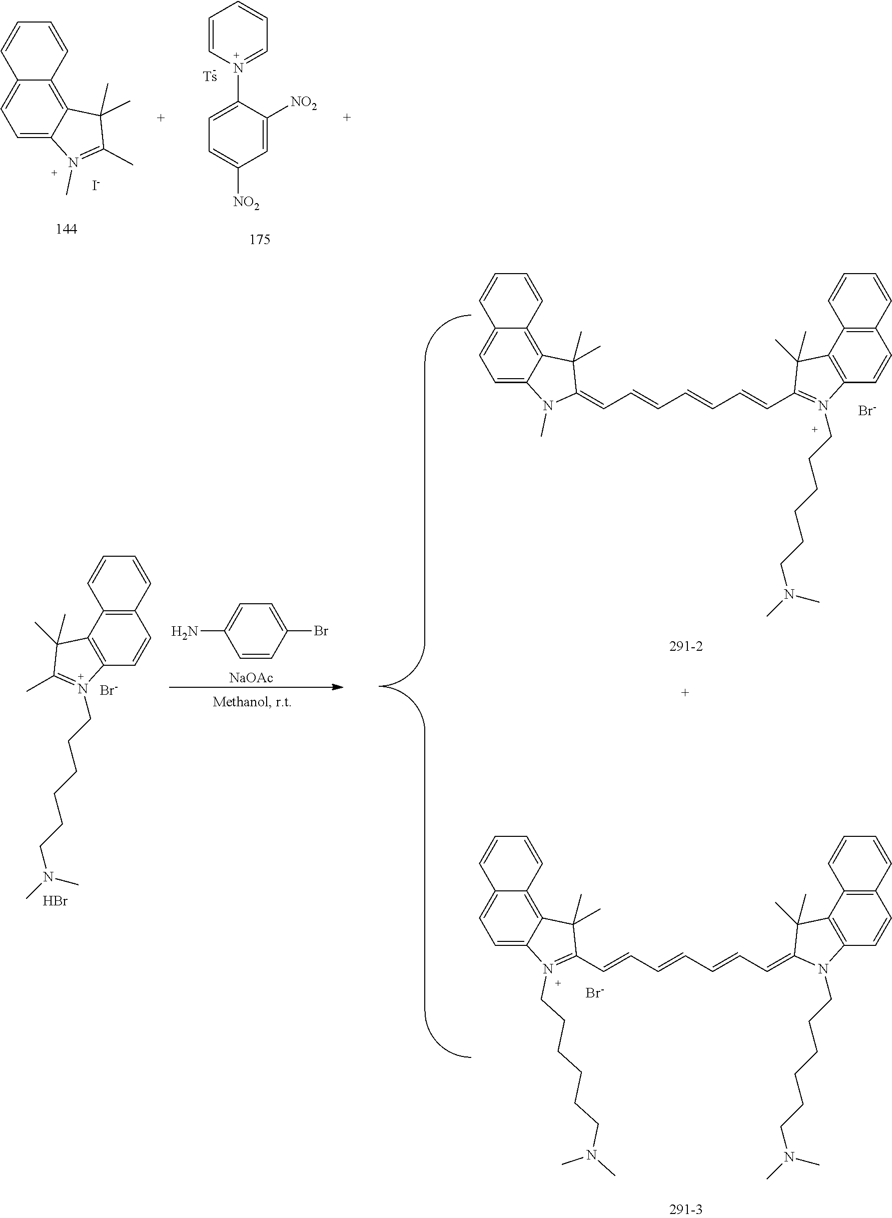

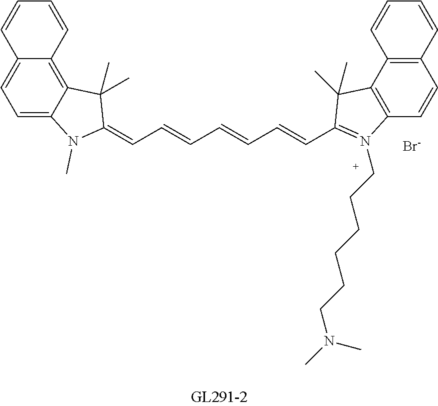

The corresponding pyridinium salt 175 (206 mg, 0.5 mmol) and 4-bromoaniline (103 mg, 0.6 mmol) were dissolved in methanol (4 mL, 7 mL/mmol), and the mixture was stirred at room temperature for 30 min. Next, a heterocyclic salt 144 (176 mg, 0.5 mmol), 288 (252 mg, 0.65 mmol) and sodium acetate (246 mg) were added, and the reaction mixture was stirred for an additional 16 h at room temperature. The crude product was purified by flash column chromatography (silica gel, dichloromethane/methanol, 20:1, 10:1, 8:1), affording GL291-2 as green solid in 19% yield. 1H NMR (600 μMHz, Methanol-d4) δ 8.13 (m, 1H), 7.91 (m, 3H), 7.54-7.48 (m, 2H), 7.37 (m, 1H), 6.52 (m, 1H), 6.24 (m, 1H), 3.38 (m, 2H), 3.02 (m, 2H), 2.79 (m, 12H), 1.89 (m, 6H), 1.81 (m, 2H), 1.64 (m, 2H), 1.42 (m, 2H), 1.33 (m, 2H). ¹³C NMR (150 MHz, Methanol-d4) δ 140.36, 139.80, 136.85, 133.37, 133.30, 131.899, 131.86, 130.91, 130.30, 130.27, 129.71, 129.68, 128.28, 128.13, 128.02, 127.31, 127.21, 124.58, 124.51, 122.99, 121.95, 121.92, 110.68, 110.52, 65.50, 57.55, 53.42, 42.17, 30.60, 27.83, 26.28, 26.12, 24.24, 20.65, 14.05. HRMS (ESI) for C₄₅H₅₂N₃⁺ [M-Br]⁺: 622.4156. Found: 622.4162.

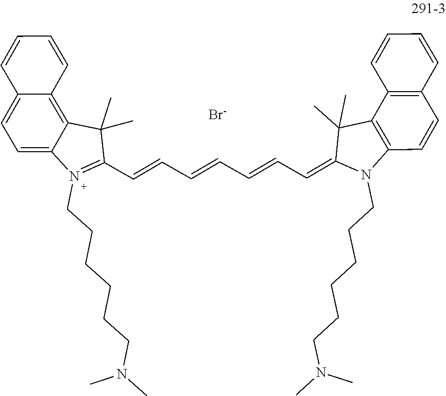

GL291-3 was afforded as a green solid in 10% yield. 1H NMR (600 μMHz, Methanol-d4) δ 8.26 (d, J=8.54 Hz, 2H), 8.08 (t, 2H), 8.03-8.00 (m, 3H), 7.97-7.94 (m, 2H), 7.78-7.77 (m, 2H), 7.66 (m, 2H), 7.60 (d, J=8.64 Hz, 2H), 7.51 (t, 2H), 6.64 (t, 2H), 6.37 (d, J=13.71 Hz, 2H), 4.25 (t, 4H), 3.03 (m, 4H), 2.80 (s, 12H), 2.03 (s, 12H), 1.93 (m, 4H), 1.73 (m, 4H), 1.59 (m, 4H), 1.50 (m, 4H). HRMS (ESI) for C₅₁H₆₇N₃⁺ [M-Br]⁺: 735.5360. Found: 735.5368.

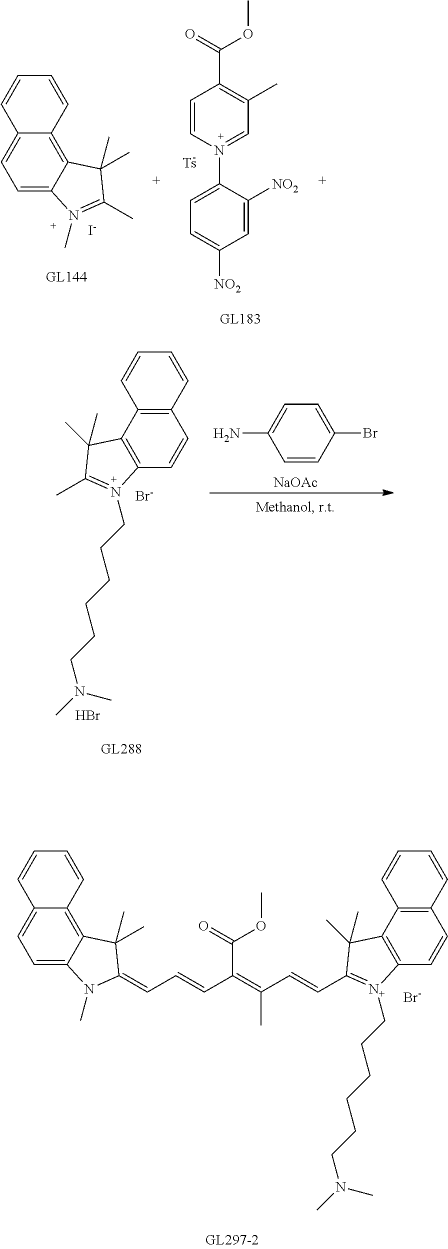

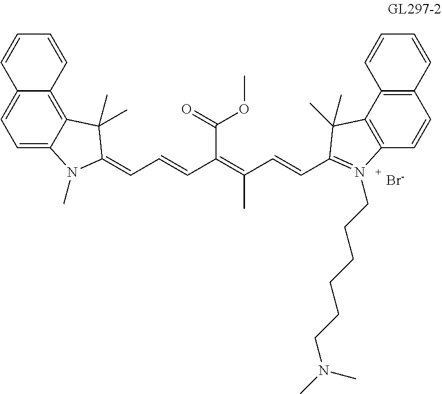

The corresponding pyridinium salt 183 (237 mg, 0.5 mmol) and 4-bromoaniline (103 mg, 0.6 mmol) were dissolved in methanol (4 mL, 7 mL/mmol), and the mixture was stirred at room temperature for 30 min. Next, a heterocyclic salt 144 (176 mg, 0.5 mmol), 288 (322 mg, 0.65 mmol) and sodium acetate (246 mg) were added, and the reaction mixture was stirred for an additional 16 h at room temperature. The crude product was purified by flash column chromatography (silica gel, dichloromethane/methanol, 30:1, 20:1, 10:1), affording GL297-2 as green solid in 22% yield. The product was found in LCMS at M: 694.4 and [M²⁺]/2: 347.8. ¹H NMR (600 μMHz, Methanol-d4) δ 8.28 (m, 2H), 8.08-8.01 (m, 4H), 7.86-7.76 (m, 2H), 7.70-7.62 (m, 4H), 7.55-7.51 (m, 2H), 6.81-6.72 (m, 1H), 6.61-6.57 (m, 1H), 6.41-6.34 (m, 1H), 4.34-4.28 (m, 2H), 4.19 (s, 3H), 3.83 (d, J=6.96 Hz, 3H), 2.90 (m, 2H), 2.68 (m, 6H), 2.21 (d, J=12.22 Hz, 3H), 1.97 (s, 12H), 1.95 (m, 2H), 1.70 (m, 2H), 1.61 (m, 2H), 1.50 (m, 2H).

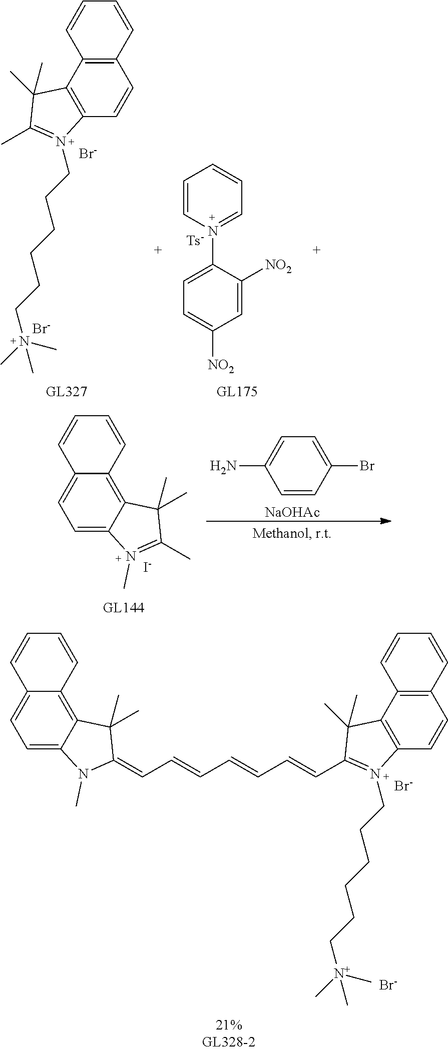

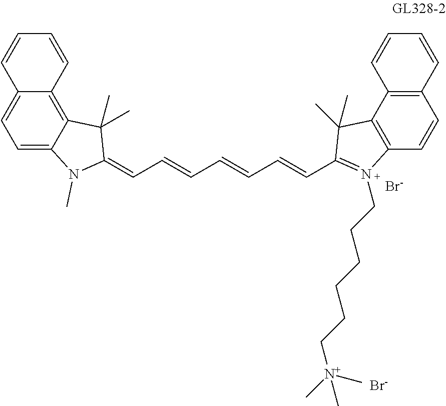

144 (105 mg, 0.3 mmol), 175 (103 mg, 0.24 mmol), 328 (154 mg, 0.3 mmol) and 4-bromoaniline (49 mg), NaOAc (116 mg) and methanol (4 mL) were mixed. The mixture was stirred at room temperature overnight. After that, the methanol was removed in a rotary evaporator. Then, the crude product was purified by flash column chromatography (silica gel, dichloromethane/methanol, 20:1 to 10:1), affording GL328-2 as a green solid in 21% yield. 1H NMR (600 μMHz, Methanol-d4) δ 8.26-8.24 (m, 2H), 8.11-7.99 (m, 6H), 7.72-7.63 (m, 3H), 7.61 (t, 2H), 7.49 (m, 2H), 6.65 (m, 2H), 6.36 (d, J=13.67 Hz, 2H), 4.25 (t, 2H), 3.76 (s, 3H), 3.41-3.38 (m, 2H), 3.15 (s, 9H), 2.01 (d, J=1.89 Hz, 12H), 1.96-1.91 (m, 2H), 1.87-1.82 (m, 2H), 1.66-1.61 (m, 2H), 1.54-1.48 (m, 2H). ¹³C NMR (150 μMHz, Methanol-d4) δ 140.35, 139.83, 133.45, 133.24, 132.05, 131.85, 130.29, 129.92, 129.72, 129.69, 128.15, 128.02, 127.99, 127.33, 127.32, 124.63, 124.51, 123.99, 121.95, 121.90, 110.61, 110.52, 66.33, 62.91, 52.23, 52.20, 52.18, 50.84, 50.67, 43.46, 30.55, 29.32, 27.21, 26.27, 26.09, 26.05, 25.79, 22.48. LCMS (ESI) for C₄₇H₅₆N₃²⁺ [M-Br]²⁺: 318.7. Found: 318.9.

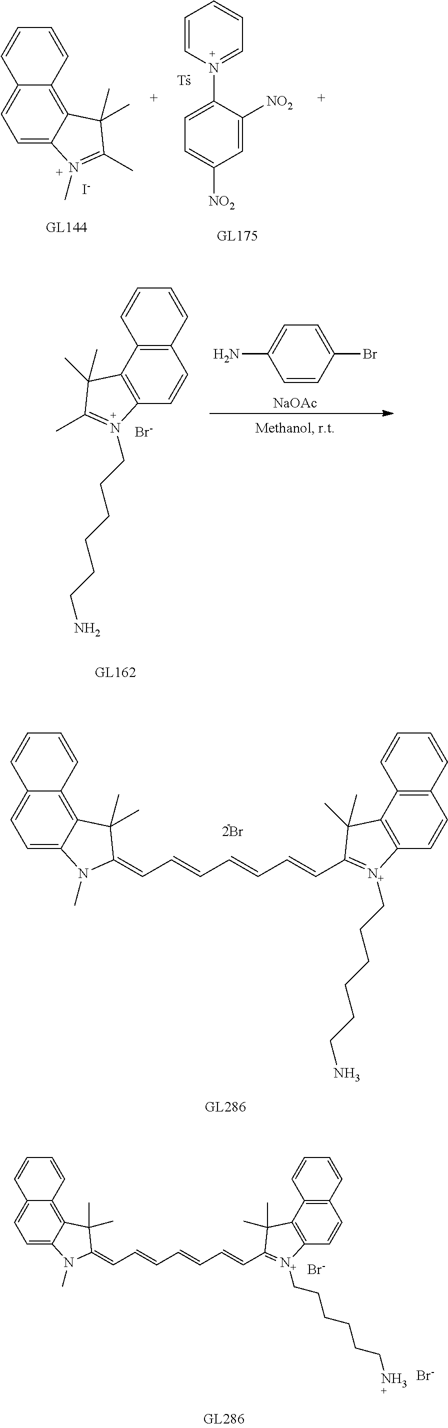

The corresponding pyridinium salt 175 (206 mg, 0.5 mmol) and 4-bromoaniline (103 mg, 0.6 mmol) were dissolved in methanol (4 mL, 7 mL/mmol), and the mixture was stirred at room temperature for 30 min. Next, a heterocyclic salt 144 (176 mg, 0.5 mmol), 162 (252 mg, 0.65 mmol) and sodium acetate (246 mg) were added, and the reaction mixture was stirred for an additional 16 h at room temperature. Afterward, Et₂O (21 mL/mmol) was added, and the mixture was placed to the freezer (−16° C.). The resulting precipitate was filtered, washed with water (2×10 mL/mmol), Et₂O (2× 10 mL/mmol), and air-dried. Then, the crude product was purified by flash column chromatography (silica gel, dichloromethane/methanol, 20:1 to 10:1), affording GL286 as a green solid in 18% yield. ¹H NMR (600 μMHz, Methanol-d4) δ 8.15-8.12 (m, 2H), 7.99-7.87 (m, 6H), 7.56-7.44 (m, 5H), 7.40-7.36 (m, 2H), 6.51 (t, 2H), 6.24-6.20 (m, 2H), 4.11 (t, 2H), 3.64 (s, 3H), 2.82 (t, 2H), 2.26 (s, 2H), 1.90 (s, 12H), 1.81 (m, 2H), 1.58 (m, 2H), 1.43 (m, 2H). HRMS (ESI) for C₄₂H₄₈N₃⁺ [M-Br—HBr]⁺: 594.3843. Found: 594.3846.

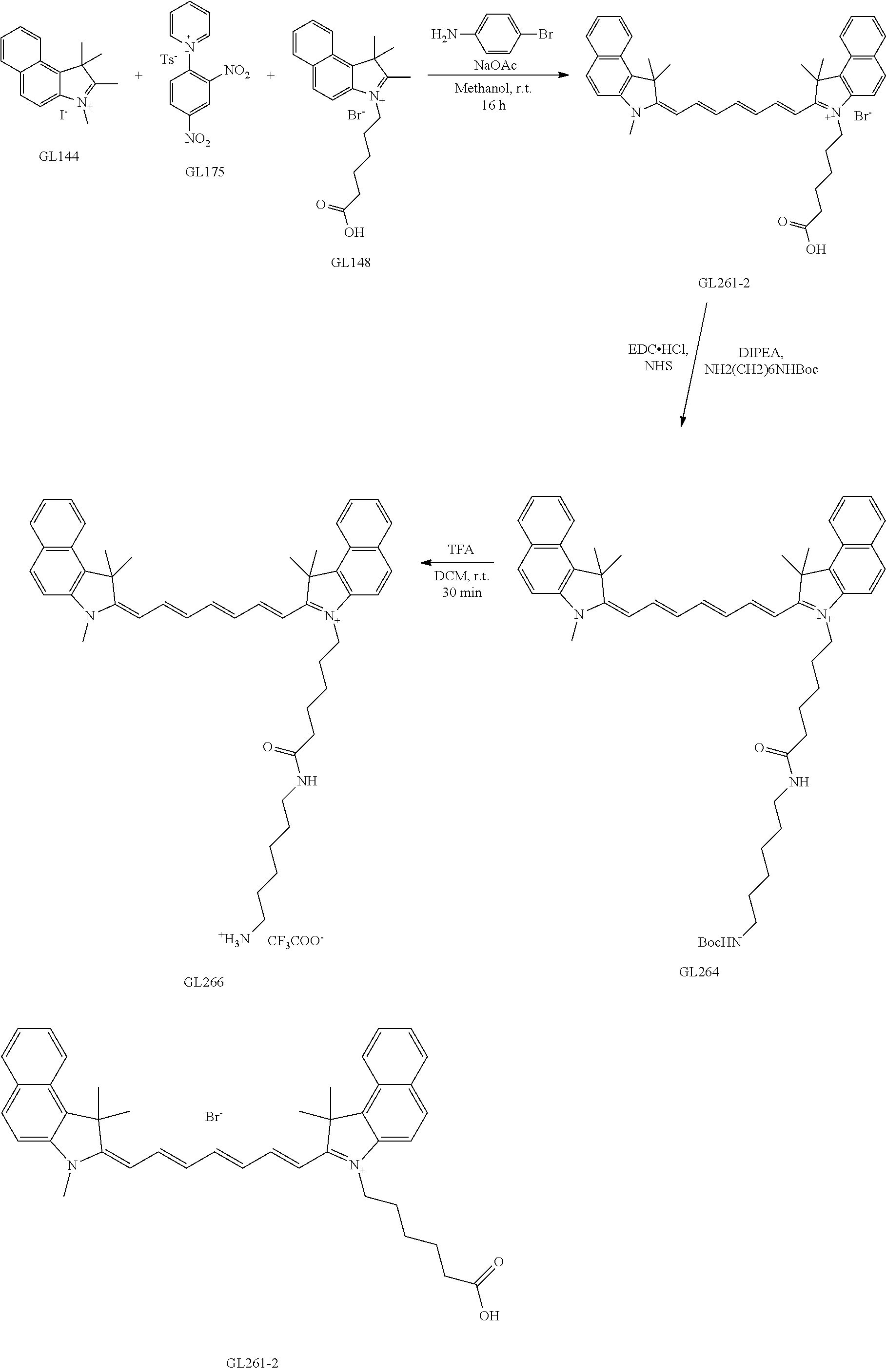

GL144 (210 mg, 0.6 mmol), GL175 (208 mg, 0.5 mmol), GL148 (265 mg, 0.6 mmol) and 4-bromoaniline (100 mg) and NaOAc (232 mg) were dissolved in methanol (4 mL), and the mixture was stirred at room temperature for overnight (16 h). The crude product was purified by flash column chromatography (silica gel, dichloromethane/methanol, 20:1 to 10:1), affording compound GL261 (87 mg, yield: 16%). 1H NMR (600 μMHz, Methanol-d4) δ 8.24 (d, J=8.86 Hz, 2H), 8.09-7.99 (m, 6H), 7.66 (t, 2H), 7.59 (t, 2H), 7.49 (t, 2H), 6.62 (t, 2H), 6.33 (m, 2H), 4.23 (t, 2H), 3.74 (s, 3H), 2.34 (t, 2H), 2.01 (s, 12H), 1.93 (m, 2H), 1.74 (m, 2H), 1.56 (m, 2H). HRMS (ESI) for C₄₂H₄₅N₂O₂⁺ [M-Br]⁺: 609.3476. Found: 609.3481.

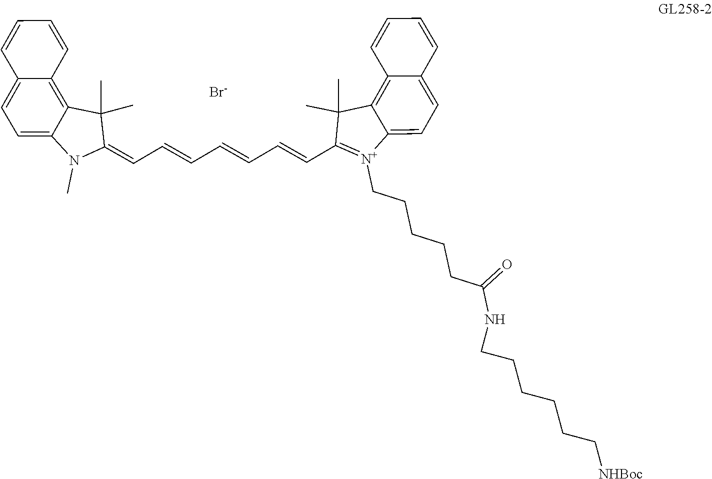 [SMILES: CN1/C(=C/C=C/C=C/C=C/C2=[N+](CCCCCC(=O)NCCCCCCNC(=O)OC(C)(C)C)c3ccc4ccccc4c3C2(C)C)C(C)(C)c2c1ccc1ccccc21 ; [Br-]]

Compound GL261 (87 mg, 0.13 mmol), 1-(3-dimethylaminopropyl)-3-ethylcarbodiimide hydrochloride (EDC·HCl, 29 mg, 0.15 mmol) and N-hydroxysuccinimide (NHS, 20 mg, 0.16 mmol) were dissolved in 5 mL DMF and the resulting mixture was stirred at room temperature under nitrogen atmosphere for 24 h in the dark. Then NH₂ (CH₂)₆NHBoc (41 mg, 0.19 mmol) and N, N-diisopropylethylamine (DIPEA, 49 mg, 0.38 mmol) were added to the reaction solution and the mixture was stirred for an additional 6 h. The crude product was purified by silica gel column chromatography with gradient elution (dichloromethane/methane of 25:1 to 15:1) to afford a purplish red solid (80 mg, 69%). ¹H NMR (600 μMHz, Methanol-d4) δ 8.13 (d, J=7.92 Hz, 2H), 7.98-7.88 (m, 6H), 7.54 (t, 2H), 7.49-7.45 (m, 3H), 7.38 (t, 2H), 6.50 (t, 2H), 6.24 (m, 2H), 4.11 (t, 2H), 3.63 (s, 3H), 2.98 (t, 2H), 2.90 (t, 2H), 2.10 (t, 2H), 1.90 (s, 12H), 1.78 (m, 2H), 1.61 (m, 2H), 1.38 (m, 2H), 1.30 (m, 14H), 1.17 (m, 6H).

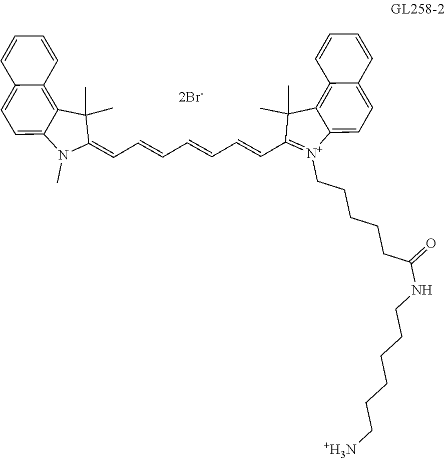 [SMILES: CN1/C(=C/C=C/C=C/C=C/C2=[N+](CCCCCC(=O)NCCCCCC[NH3+])c3ccc4ccccc4c3C2(C)C)C(C)(C)c2c1ccc1ccccc21 ; "2Br-"]

In an 8 ml vial, 80 mg of Boc compound was dissolved in 10 mL DCM, and 1 ml of TFA was added dropwise. The solution was stirred for 30 min, then, the excess of TFA was removed by vacuum, affording GL258-2 as a green solid. 1H NMR (600 μMHz, Methanol-d4) δ 8.13 (d, J=8.16 Hz, 2H), 7.98-7.85 (m, 6H), 7.57-7.45 (m, 5H), 7.38 (m, 2H), 6.50 (t, 2H), 6.23 (t, 2H), 4.10 (t, 2H), 3.63 (s, 3H), 3.01 (t, 2H), 2.78 (t, 2H), 2.02 (t, 2H), 1.89 (s, 12H), 1.77 (m, 2H), 1.60 (m, 2H), 1.52 (m, 2H), 1.42-1.32 (m, 4H), 1.29-1.19 (m, 4H).

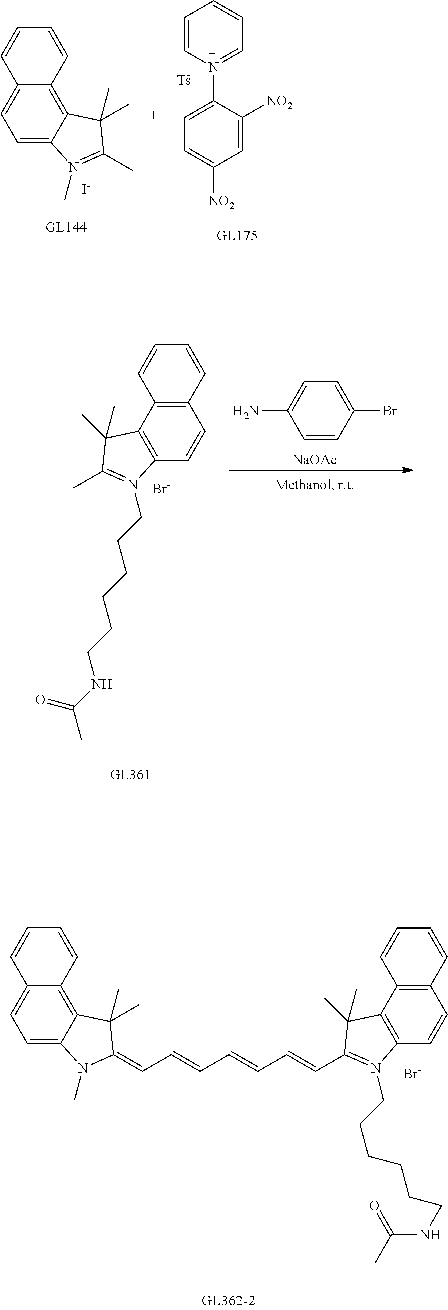

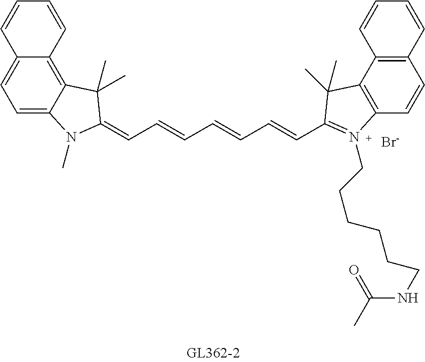

The corresponding pyridinium salt GL175 (103 mg, 0.24 mmol) and 4-bromoaniline (49 mg, 0.29 mmol) were dissolved in methanol (4 mL), and the mixture was stirred at room temperature for 30 min. Next, a heterocyclic salt GL144 (105 mg, 0.3 mmol), GL361 (129 mg, 0.3 mmol) and sodium acetate (116 mg) were added, and the reaction mixture was stirred for an additional 16 h at room temperature. The crude product was purified by flash column chromatography (silica gel, dichloromethane/methanol, 20:1, 10:1), affording GL362-2 as a green solid in 23% yield. ¹H NMR (600 μMHz, Methanol-d4) δ 8.24 (d, J=8.51 Hz, 2H), 8.09-7.99 (m, 6H), 7.70-7.57 (m, 5H), 7.49 (t, 2H), 6.62 (t, 2H), 6.34 (d, J=14.40 Hz, 2H), 4.22 (t, 2H), 3.74 (s, 3H), 3.18 (t, 2H), 2.01 (s, 12H), 1.93 (s, 3H), 1.88 (m, 2H), 1.55 (m, 4H), 1.46 (m, 2H). ¹³C NMR (150 μMHz, Methanol-d4) δ 171.84, 140.40, 139.78, 133.39, 133.28, 131.98, 131.92, 130.32, 130.26, 129.71, 128.11, 128.05, 127.31, 125.57, 124.56, 121.92, 110.61, 110.47, 53.40, 50.76, 50.73, 48.47, 48.22, 43.60, 38.88, 30.44, 28.82, 27.28, 26.26, 26.23, 26.11, 26.08, 21.16. HRMS (ESI) for C₄₄H₅₀N₃O⁺ [M-Br]⁺: 636.3948. Found: 636.3954.

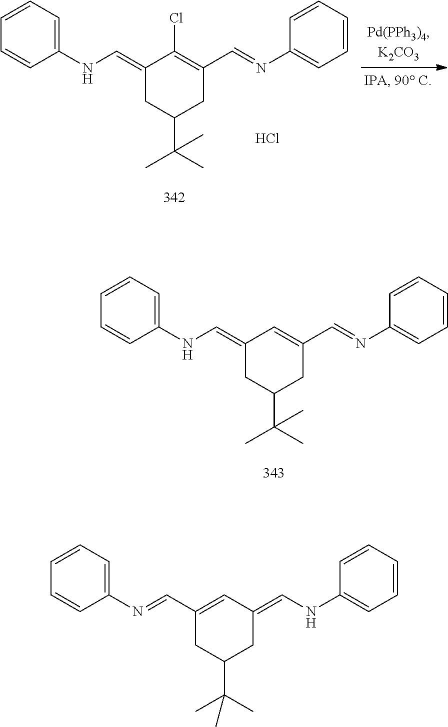 [SMILES: CC(C)(C)C1CC(/C=N/c2ccccc2)=C(Cl)C(=C/Nc2ccccc2)/C1 ; Cl ; CC(C)(C)C1CC(/C=N/c2ccccc2)=CC(=C/Nc2ccccc2)/C1 ; CC(C)(C)C1CC(/C=N/c2ccccc2)=CC(=C/Nc2ccccc2)/C1]

GL342 (830 mg, 2 mmol), Pd(PPh₃)₄ (250 mg, 0.2 mmol), and K₂CO₃ (1.1 mg, 6 mmol) were mixed in 15 mL of isopropanol, and then stirred and heated at 90° C. for overnight. The dark solution was changed to yellow. After cooling down to room temperature, the solvents were removed by rotary evaporation. The crude product was purified by flash silica gel column (Hexane and ethyl acetate 10:1), affording the orange solid GL343. LCMS (ESI) for C₂₄H₂₈N₂ [M+H]⁺: 345.23. Found: 345.3.

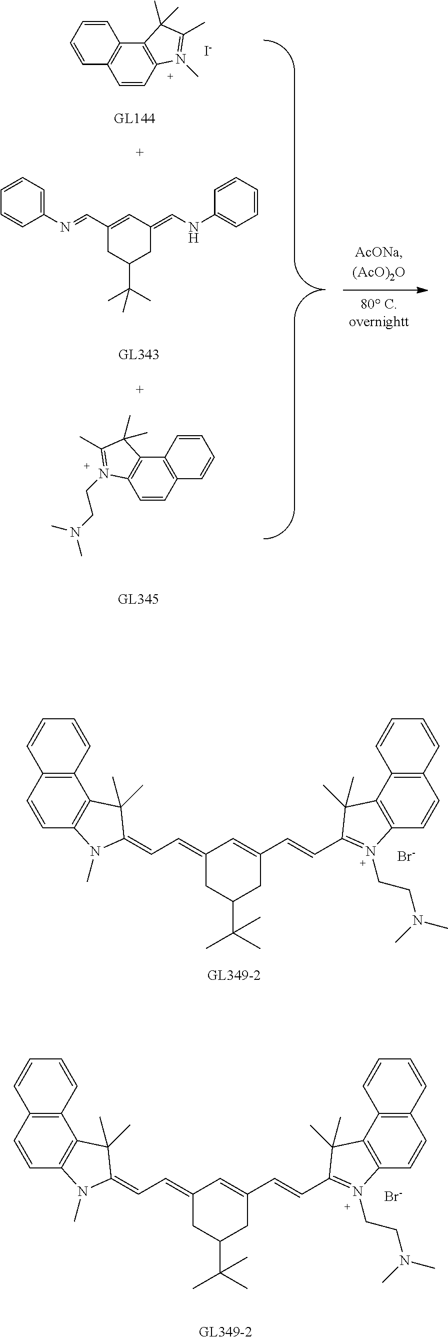

GL144 (176 mg, 0.5 mmol), GL343 (172 mg, 0.5 mmol), compound GL345 (286 mg, 0.65 mmol) and sodium acetate (246 mg, 3 mmol) and acetic anhydride (4 ml) were stirred and heated at 80° C. for overnight. The dark solution was cooled down and poured into ether. The green solid was filtered and collected. The crude product was purified by silica gel column (DCM and Methanol 10:1), obtaining GL349-2 as a green solid (yield: 10%). 1H NMR (600 MHz, Methanol-d4) δ 8.27 (d, J=8.82 Hz, 1H), 8.23 (d, J=8.82 Hz, 1H), 8.07-7.98 (m, 5H), 7.85 (d, J=13.24 Hz, 1H), 7.69-7.59 (m, 3H), 7.56-7.46 (m, 4H), 6.32 (d, J=14.42 Hz, 1H), 6.23 (d, J=14.22 Hz, 1H), 4.36 (t, 2H), 3.82 (s, 3H), 2.92 (m, 2H), 2.84 (t, 2H), 2.50 (s, 6H), 2.05 (m, 12H), 2.01 (s, 4H), 1.15 (s, 9H). ¹³C NMR (150 μMHz, Methanol-d4) δ 174.35, 149.04, 140.27, 139.76, 132.16, 131.73, 130.34, 130.25, 129.75, 128.24, 127.94, 127.39, 127.22, 124.79, 124.22, 122.02, 121.84, 110.62, 110.07, 100.05, 98.09, 54.94, 51.06, 50.32, 44.40, 43.67, 41.11, 32.06, 30.72, 26.55, 26.29, 26.14, 26.06, 25.92. 19.64. HRMS (ESI) for C₄₇H₅₆N₃⁺ [M-Br]⁺: 662.4469. Found: 662.4474.

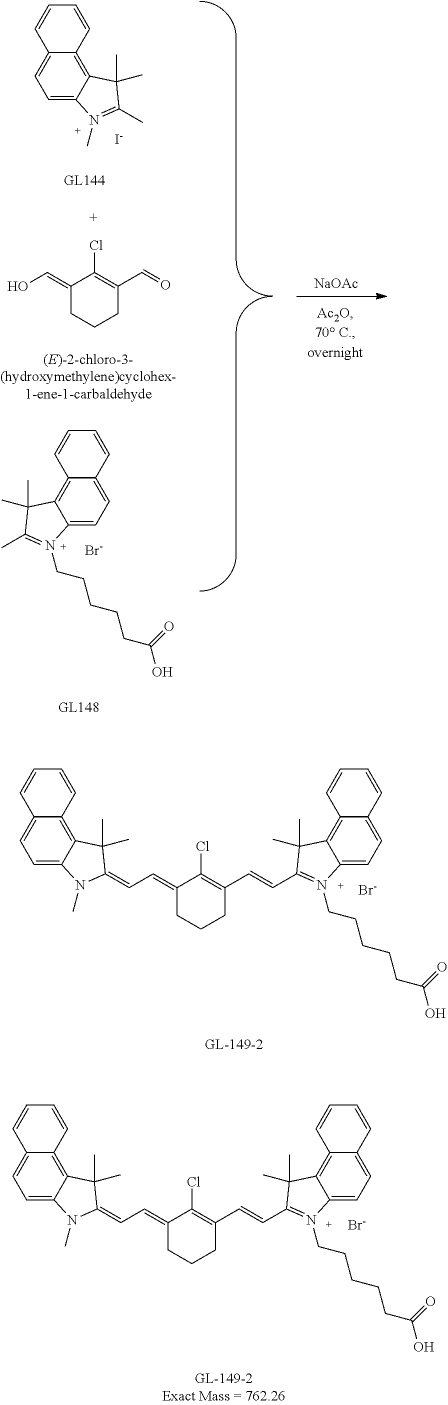

GL144 (176 mg, 0.5 mmol), GL148 (202.2 mg, 0.5 mmol), (E)-2-chloro-3-(hydroxymethylene)cyclohex-1-ene-1-carbaldehyde (86.3 mg, 0.5 mmol) and sodium acetate (86 mg, 1.05 mmol) and Acetic anhydride (5 ml) were stirred and heated at 70° C. for overnight. The dark solution was cooled down and poured into ether. The green solid was filtered and collected. The crude product was purified by silica gel column (DCM and Methanol 10:1), obtaining GL149-2 as a green solid (yield: 26.5%). 1H NMR (600 μMHz, Methanol-d4) δ 8.58 (t, 1H), 8.30 (d, J=7.61 Hz, 1H), 8.07-8.02 (m, 2H), 7.69-7.63 (m, 2H), 7.53 (t, 1H), 6.35 (d, J=12.17 Hz, 1H), 4.33 (t, 1H), 3.83 (s, 3H), 2.80 (s, 2H), 2.34 (t, 1H), 2.12 (s, 6H), 2.04 (m, 2H), 1.96 (m, 2H), 1.75 (m, 2H), 1.57 (m, 2H). ¹³C NMR (150 μMHz, Methanol-d4) δ 176.40, 174.90, 174.24, 173.96, 149.19, 149.11, 143.33, 143.18, 143.05, 142.97, 140.32, 139.71, 133.83, 133.81, 132.23, 132.15, 130.49, 130.44, 129.76, 129.34, 128.05, 127.95, 127.44, 127.43, 126.61, 126.57, 126.49, 126.43, 124.92, 124.86, 124.83, 122.04, 110.75, 110.64, 100.80, 100.35, 51.06, 48.16, 43.83, 33.65, 33.07, 30.72, 26.99, 26.48, 26.38, 26.02, 25.96, 25.92, 24.46, 24.24, 20.79. HRMS (ESI) for C₄₅H₄₈ClN₂O₂⁺ [M-Br]⁺: 683.3390. Found: 683.3312.

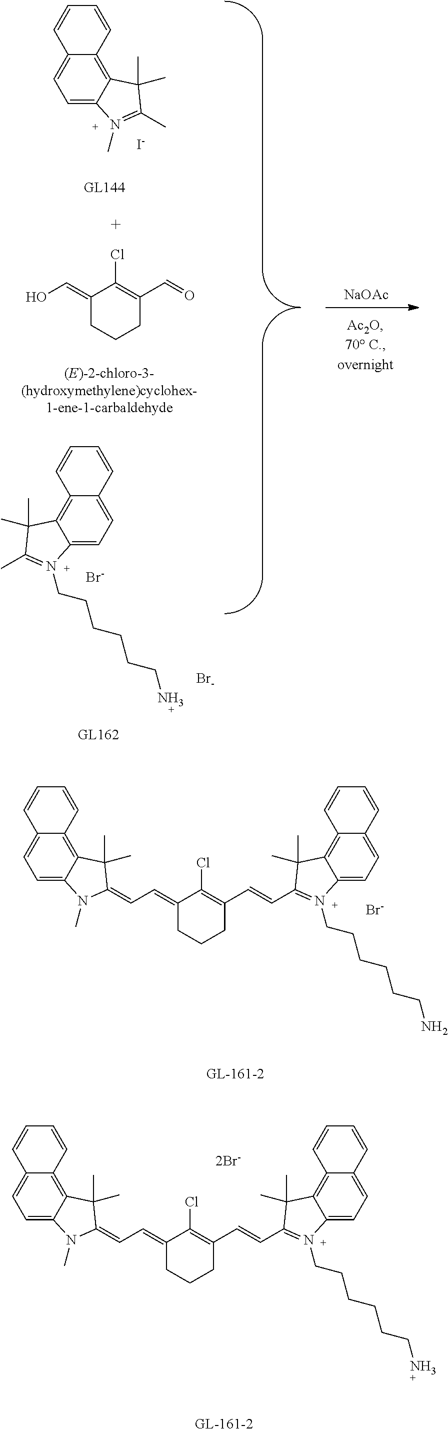

GL144 (176 mg, 0.5 mmol), GL162 (223.5 mg, 0.5 mmol), (E)-2-chloro-3-(hydroxymethylene)cyclohex-1-ene-1-carbaldehyde (86.3 mg, 0.5 mmol) and sodium acetate 5 (86 mg, 1.05 mmol) and Acetic anhydride (3 ml) were stirred and heated at 70° C. for overnight. The dark solution was cooled down and poured into ether. The green solid was filtered and collected. The crude product was purified by silica gel column (DCM and Methanol 10:1), obtaining GL161-2 as a green solid (yield: 26.5%). 1H NMR (600 μMHz, Methanol-d4) 8.62-8.56 (m, 2H), 8.30 (m, 2H), 8.08-8.02 (m, 4H), 7.70-7.63 (m, 4H), 7.53 (m, 2H), 6.35 (m, 2H), 4.32 (t, 2H), 3.83 (s, 3H), 3.18 (t, 2H), 2.80 (m, 4H), 2.06 (d, 12H), 2.03 (m, 2H), 1.95 (m, 2H), 1.55 (m, 4H), 1.48 (m, 2H).

TABLE 1
Condition optimizations of charged pi-conjugated compounds key
intermediate 2

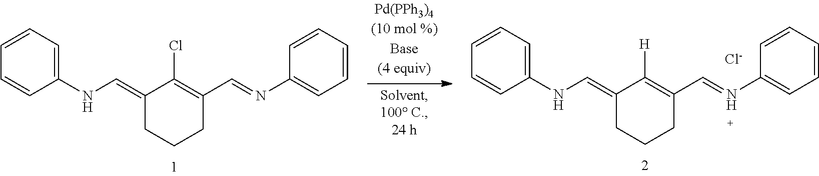 [SMILES: ClC1=C(/C=N/c2ccccc2)CCC/C1=C\Nc1ccccc1 ; C1=C(/C=N/c2ccccc2)CCC/C1=C\Nc1ccccc1 ; [Cl-]]

| Entry^{[a]} | Base | Solvent | Yield (%)^{[b]} |
|---|---|---|---|
| 1 | K₃PO₄ | DMF/H₂O (5:1) | 31 |
| 2 | K₂HPO₄ | DMF/H₂O (5:1) | 30 |
| 3 | KF | DMF/H₂O (5:1) | 25 |
| 4 | K₂CO₃ | DMF/H₂O (5:1) | 32 |
| 5 | NaOtBu | DMF/H₂O (5:1) | 0 |
| 6 | NaOAc | DMF/H₂O (5:1) | 0 |
| 7 | CsF | DMF/H₂O (5:1) | 3 |
| 8 | Cs₂CO₃ | DMF/H₂O (5:1) | 10 |
| 9 | K₂CO₃ | DMF/H₂O/IPA | 35 |
| | | (5:1:1) | |
| 10 | K₂CO₃ | EtOH | 37 |
| 11 | K₂CO₃ | IPA | 66 (61)^{[c]} |

^{[a]}The reactions were carried out under the following conditions: 1) 1 (0.25 mmol), Pd(PPh₃)₄ (10 mol %), Base (4 equiv.) in solvent at 100° C. for 24 h;
^{[b]}Yields were determined by HPLC on a C18 reverse-phase column;
^{[c]}Isolated yield.

General procedure: A screw-capped vial was added with 1 (0.25 mmol), Pd(PPh₃)₄ (0.025 mmol, 10 mol %), Base (1 mmol, 4 equiv.). The vial was sealed with a PTFE septum and then evacuated and backfilled with N₂ three times, followed by the addition of solvent via syringe and vigorous stirring. The sealed reaction was heated to 100° C. in an oil bath heating for 24 h; then the reaction was cooled to room temperature and concentrated under reduced pressure, followed by 6M HCl at 0° C. and stirred for 10 mins at room temperature. The precipitate was filtered, washed with H₂O, Et₂O and acetone, and dried in vacuo to provide compound 2 as a dark red solid.

N—((E)-(3-((E)-(phenylimino)methyl) cyclohex-2-en-1-ylidene)methyl) aniline (2): ¹H-NMR (600 μMHz, CD₃OD)_{δ 8.14} (s, 2H), 7.79 (s, 1H), 7.49-7.39 (m, 8H), 7.28-7.23 (m, 2H), 2.58 (t, J=6.2 Hz, 4H), 1.99-1.93 (m, 2H). ¹³C NMR (151 μMHz, CD₃OD) δ 163.58, 153.18, 140.74, 130.98, 127.16, 119.97, 119.18, 22.96, 21.58. HRMS (ESI) calculated for [M+H, C₂₀H₂₁N₂]⁺: 289.1626, found: 289.1707.

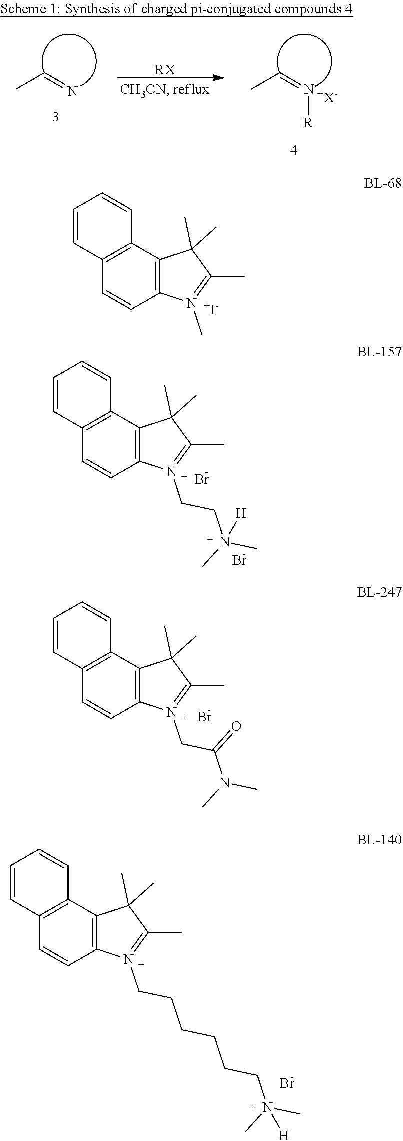

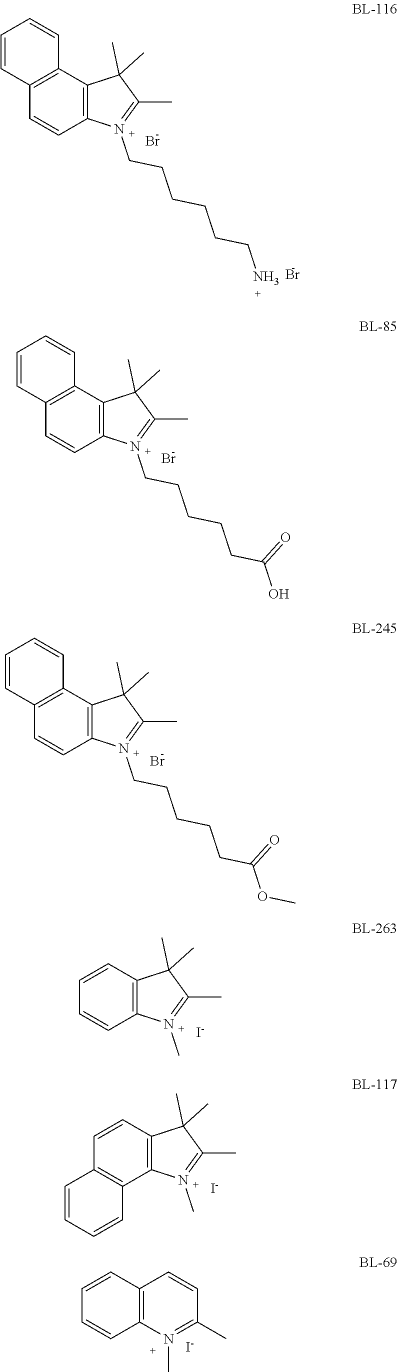

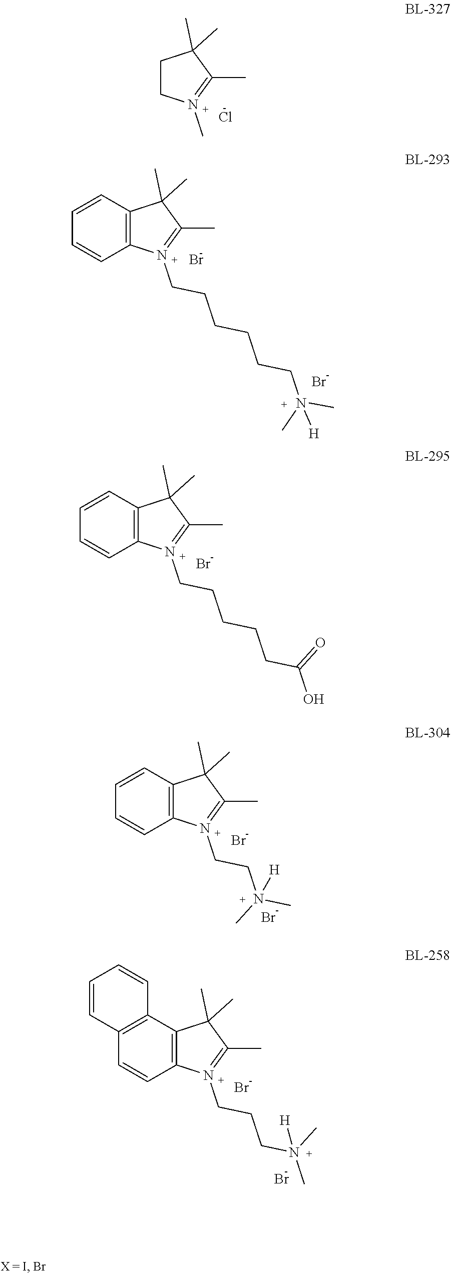

General procedure: To a screwed-capped vial charged with compound 3 (1 equiv.) and alkyl halides (1.5 equiv.) were heated to reflux in CH₃CN until compound 3 consumed all. Subsequently, the mixture was cooled to room temperature; then, diethyl ether was added to precipitate the product. That product was collected by filtration and washed with diethyl ether to obtain compound 4.

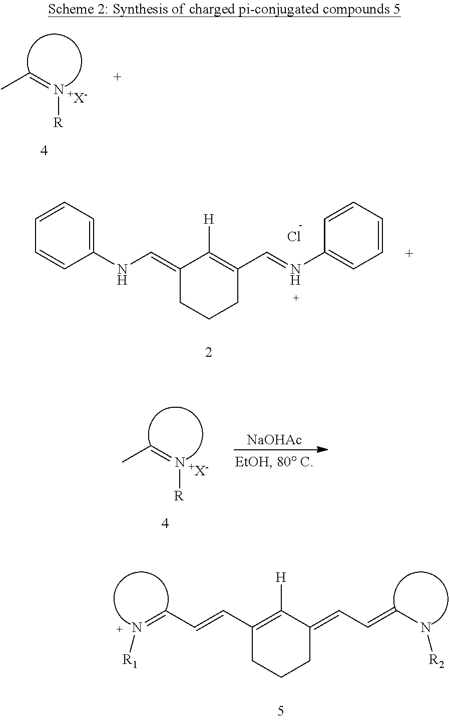

General Synthetic Procedure: All glassware was oven-dried overnight prior to use. All reactions were carried out under an N₂ atmosphere unless otherwise noted. All other chemicals were purchased from commercial suppliers and used without further purification.

General procedure: To a screwed-capped vial charged with compound 3 (1 equiv.) and alkyl halides (1.5 equiv.) were heated to reflux in CH₃CN until compound 3 consumed all. Subsequently, the mixture was cooled to room temperature; then, diethyl ether was added to precipitate the product. That product was collected by filtration and washed with diethyl ether to obtain compound 4.

Procedure b: A screw-capped vial was added with compound 4 (1 equiv.), 2 (1 equiv.), 4 (1 equiv.) and NaOAc (3 equiv.) and dissolved in absolute ethanol. The mixture was heated at 80° C. overnight under N₂ atmosphere. The final product was purified by silica column chromatography to obtain compound 5.

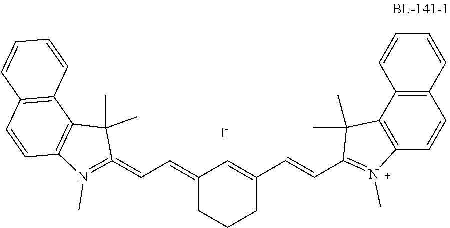

1,1,3-trimethyl-2-((E)-2-((E)-3-((E)-2-(1,1,3-trimethyl-1,3-dihydro-2H-benzo[e]indol-2-ylidene)ethylidene)cyclohex-1-en-1-yl) vinyl)-1H-benzo[e]indol-3-ium (BL-141-1): Prepared according to the general procedure from BL-68 (176 mg, 0.5 mmol, 1 equiv.) and 2 (81 mg, 0.25 mmol, 1 equiv.). Yield: 88% yield (149 mg). ¹H-NMR (600 μMHz, CD₃OD) δ 8.24-8.21 (m, 2H), 8.00 (d, J=8.8 Hz, 2H), 7.97 (d, J=8.6 Hz, 2H), 7.87 (d, J=14.2 Hz, 2H), 7.64-7.60 (m, 2H), 7.57 (d, J=8.8 Hz, 2H), 7.53 (s, 1H), 7.48-7.44 (m, 2H), 6.19 (d, J=14.1 Hz, 2H), 3.72 (s, 6H), 2.61 (t, J=6.1 Hz, 4H), 2.03-1.95 (m, 14H). HRMS (ESI) calculated for [M, C₄₀H₄₁N₂]⁺: 549.3264, found: 549.3275.

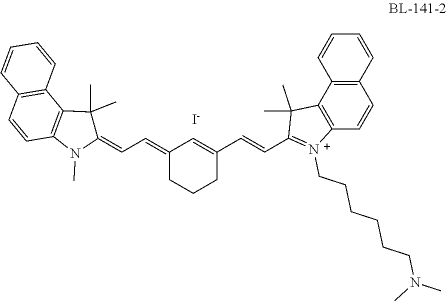 [SMILES: CN(C)CCCCCC[N+]1=C(/C=C/C2=CC(=C/C=C3/N(C)c4ccc5ccccc5c4C3(C)C)/CCC2)C(C)(C)c2c1ccc1ccccc21 ; [I-]]

3-(6-(dimethylamino) hexyl)-1,1-dimethyl-2-((E)-2-((E)-3-((E)-2-(1,1,3-trimethyl-1,3-dihydro-2H-benzo[e]indol-2-ylidene)ethylidene)cyclohex-1-en-1-yl) vinyl)-1H-benzo[e]indol-3-ium (BL-141-2): Prepared according to the general procedure from BL-68 (176 mg, 0.5 mmol, 1 equiv.), 2 (162 mg, 0.5 mmol, 1 equiv.) and BL-140 (248 mg, 0.5 mmol, 1 equiv.). Yield: 14% yield (45 mg). ¹H-NMR (600 μMHz, CD₃OD) δ 8.27-8.21 (m, 2H), 8.05-7.96 (m, 4H), 7.93-7.89 (m, 1H), 7.85 (d, J=14.0 Hz, 1H), 7.67-7.61 (m, 2H), 7.60 (d, J=8.8 Hz, 1H), 7.57 (d, J=8.8 Hz, 1H), 7.52 (s, 1H), 7.50-7.45 (m, 2H), 6.21 (dd, J=23.7, 14.1 Hz, 2H), 4.24 (t, J=7.4 Hz, 2H), 3.75 (s, 3H), 3.04 (t, J=9.0 Hz, 2H), 2.80 (s, 6H), 2.63-2.60 (m, 4H), 2.06-1.90 (m, 14H), 1.75-1.70 (m, 2H), 1.59-1.54 (m, 2H), 1.51-1.47 (m, 2H). HRMS (ESI) calculated for [M, C₄₇H₅₆N₃]⁺: 662.4469, found: 662.4476.

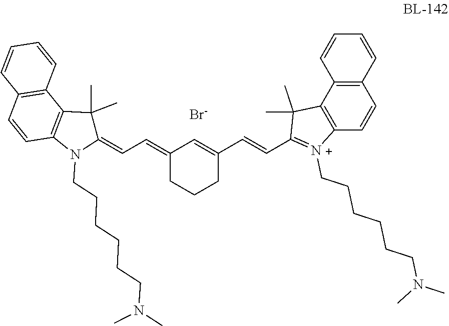 [SMILES: CN(C)CCCCCCN1/C(=C/C=C2C=C(/C=C/C3=[N+](CCCCCCN(C)C)c4ccc5ccccc5c4C3(C)C)CCC/2)C(C)(C)c2c1ccc1ccccc21 ; [Br-]]

3-(6-(dimethylamino) hexyl)-2-((E)-2-((E)-3-((E)-2-(3-(6-(dimethylamino) hexyl)-1,1-dimethyl-1,3-dihydro-2H-benzo[e]indol-2-ylidene)ethylidene)cyclohex-1-en-1-yl) vinyl)-1,1-dimethyl-1H-benzo[e]indol-3-ium (BL-142): Prepared according to the general procedure from 2 (81 mg, 0.25 mmol, 1 equiv.) and BL-140 (248 mg, 0.5 mmol, 2 equiv.). Yield: 20% yield (40 mg). ¹H-NMR (600 μMHz, CD₃OD) δ 8.25-8.22 (m, 2H), 8.03-7.96 (m, 4H), 7.89 (d, J=14.1 Hz, 2H), 7.66-7.62 (m, 2H), 7.60 (d, J=8.8 Hz, 2H), 7.56 (s, 1H), 7.50-7.45 (m, 2H), 6.24 (d, J=14.1 Hz, 2H), 4.27 (t, J=7.4 Hz, 4H), 4.27 (t, J=7.4 Hz, 4H), 2.82 (s, 12H), 2.63 (t, J=6.2 Hz, 4H), 2.05-1.96 (m, 12H), 1.95-1.90 (m, 6H), 1.76-1.72 (m, 4H), 1.60-1.55 (m, 4H), 1.52-1.47 (m, 4H). HRMS (ESI) calculated for [M, C₅₄H₇₁N₄]⁺: 775.5673, found: 775.5662.

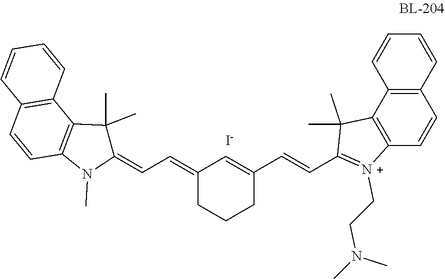 [SMILES: CN(C)CC[N+]1=C(/C=C/C2=CC(=C/C=C3/N(C)c4ccc5ccccc5c4C3(C)C)/CCC2)C(C)(C)c2c1ccc1ccccc21 ; [I-]]

3-(2-(dimethylamino)ethyl)-1,1-dimethyl-2-((E)-2-((E)-3-((E)-2-(1,1,3-trimethyl-1,3-dihydro-2/I-benzo[e]indol-2-ylidene)ethylidene)cyclohex-1-en-1-yl) vinyl)-1H-benzo[e]indol-3-ium (BL-204): Prepared according to the general procedure from BL-68 (70 mg, 0.2 mmol, 1 equiv.), 2 (65 mg, 0.2 mmol, 1 equiv.) and BL157 (107 mg, 0.2 mmol, 1 equiv.). Yield: 18% yield (26 mg). ¹H-NMR (600 μMHz, CD₃OD) δ 8.25 (d, J=8.6 Hz, 1H), 8.22 (d, J=8.7 Hz, 1H), 8.05-7.90 (m, 5H), 7.82 (d, J=13.9 Hz, 1H), 7.67-7.59 (m, 3H), 7.56-7.44 (m, 4H), 6.28 (d, J=14.2 Hz, 1H), 6.21 (d, J=14.1 Hz, 1H), 4.30 (t, J=7.4 Hz, 2H), 3.77 (s, 3H), 2.77 (t, J=7.4 Hz, 2H), 2.66-2.59 (m, 4H), 2.43 (s, 6H), 2.06-1.95 (m, 14H). HRMS (ESI) calculated for [M, C₄₃H₄₈N₃]⁺: 606.3843, found: 606.3847.

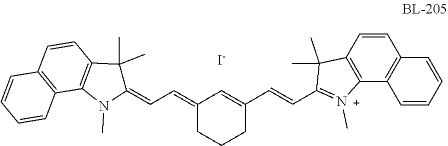 [SMILES: CN1/C(=C/C=C2C=C(/C=C/C3=[N+](C)c4c(ccc5ccccc45)C3(C)C)CCC/2)C(C)(C)c2ccc3ccccc3c21 ; [I-]]

1,3,3-trimethyl-2-((E)-2-((E)-3-((E)-2-(1,3,3-trimethyl-1,3-dihydro-2H-benzo[g]indol-2-ylidene)ethylidene)cyclohex-1-en-1-yl) vinyl)-3H-benzo[g]indol-1-ium (BL-205): Prepared according to the general procedure from BL-117 (70 mg, 0.2 mmol, 2 equiv.) and 2 (33 mg, 0.1 mmol, 1 equiv.). Yield: 74% yield (50 mg). ¹H-NMR (600 μMHz, CD₃OD) δ 8.59 (d, J=8.7 Hz, 2H), 7.99 (d, J=8.2 Hz, 2H), 7.82 (d, J=8.9 Hz, 4H), 7.67-7.59 (m, 4H), 7.58-7.52 (m, 2H), 7.50 (s, 1H), 6.33 (d, J=14.0 Hz, 2H), 4.18 (s, 6H), 2.63 (t, J=6.2 Hz, 4H), 2.02-1.96 (m, 2H), 1.77 (s, 12H). HRMS (ESI) calculated for [M, C₄₀H₄₁N₂]⁺: 549.3264, found: 549.3247.

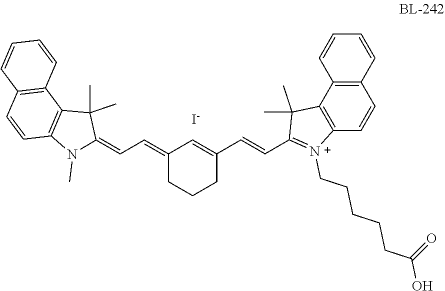 [SMILES: CN1/C(=C/C=C2C=C(/C=C/C3=[N+](CCCCCC(=O)O)c4ccc5ccccc5c4C3(C)C)CCC/2)C(C)(C)c2c1ccc1ccccc21 ; [I-]]

3-(5-carboxypentyl)-1,1-dimethyl-2-((E)-2-((E)-3-((E)-2-(1,1,3-trimethyl-1,3-dihydro-2H-benzo[e]indol-2-ylidene)ethylidene)cyclohex-1-en-1-yl) vinyl)-1H-benzo[e]indol-3-ium (BL-242): Prepared according to the general procedure from BL-68 (117 mg, 0.33 mmol, 1 equiv.), 2 (107 mg, 0.33 mmol, 1 equiv.) and BL-85 (134 mg, 0.33 mmol, 1 equiv.). Yield: 18% yield (130 mg). ¹H-NMR (600 μMHz, CD₃OD) δ 8.12 (d, J=8.5 Hz, 2H), 7.91-7.85 (m, 4H), 7.84-7.69 (m, 2H), 7.55-7.51 (m, 2H), 7.50-7.32 (m, 5H), 6.21-5.95 (m, 2H), 4.20-4.06 (m, 2H), 3.63 (s, 3H), 2.68-2.30 (m, 4H), 2.23 (t, J=7.3 Hz, 2H), 1.99-1.84 (m, 14H), 1.83-1.77 (m, 2H), 1.65-1.60 (m, 2H), 1.48-1.41 (m, 2H); ¹³C-NMR (151 μMHz, CD₃OD) δ 177.49, 174.88, 173.98, 163.31, 163.08, 162.84, 149.06, 148.67, 141.83, 141.22, 134.55, 133.35, 133.29, 132.30, 131.66, 131.61, 131.10, 129.68, 129.53, 129.46, 128.66, 125.84, 124.39, 123.33, 113.51, 111.95, 111.83, 100.54, 100.28, 54.81, 52.05, 44.83, 34.81, 31.83, 28.22, 27.60, 27.50, 27.41, 25.78, 25.02, 22.78, 22.07. HRMS (ESI) calculated for [M, C₄₅H₄₉N₂O₂]⁺: 649.3789, found: 649.3793.

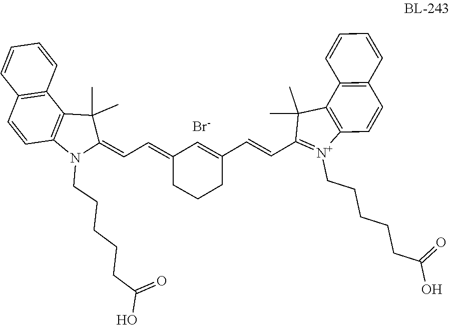 [SMILES: CC1(C)C(/C=C/C2=CC(=C/C=C3/N(CCCCCC(=O)O)c4ccc5ccccc5c4C3(C)C)/CCC2)=[N+](CCCCCC(=O)O)c2ccc3ccccc3c21 ; [Br-]]

3-(5-carboxypentyl)-2-((E)-2-((E)-3-((E)-2-(3-(5-carboxypentyl)-1,1-dimethyl-1,3-dihydro-2H-benzo[e]indol-2-ylidene)ethylidene)cyclohex-1-en-1-yl) vinyl)-1,1-dimethyl-1H-benzo[e]indol-3-ium (BL-243): Prepared according to the general procedure from 2 (33 mg, 0.1 mmol, 1 equiv.) and BL-85 (81 mg, 0.2 mmol, 2 equiv.). Yield: 45% yield (39 mg). ¹H-NMR (600 μMHz, CD₃OD) δ 8.23 (d, J=8.5 Hz, 2H), 8.00 (d, J=8.9 Hz, 2H), 7.98 (d, J=8.0 Hz, 2H), 7.86 (d, J=14.0 Hz, 2H), 7.65-7.61 (m, 2H), 7.57 (d, J=8.8 Hz, 2H), 7.50-7.44 (m, 3H), 6.22 (d, J=14.1 Hz, 2H), 4.23 (t, J=7.5 Hz, 4H), 2.61 (t, J=6.2 Hz, 4H), 2.25 (t, J=7.4 Hz, 4H), 2.03-1.96 (m, 12H), 1.93-1.87 (m, 4H), 1.75-1.70 (m, 4H), 1.56-1.50 (m, 4H). HRMS (ESI) calculated for [M, C₅₀H₅₇N₂O₄]⁺: 749.4313, found: 749.4312.

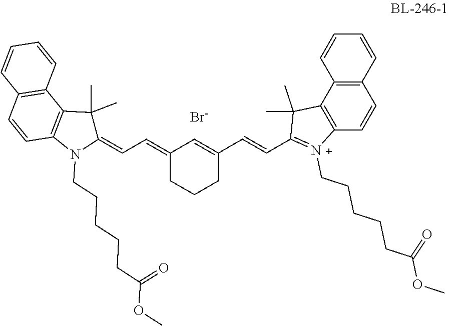 [SMILES: COC(=O)CCCCCN1/C(=C/C=C2C=C(/C=C/C3=[N+](CCCCCC(=O)OC)c4ccc5ccccc5c4C3(C)C)CCC/2)C(C)(C)c2c1ccc1ccccc21 ; [Br-]]

3-(6-methoxy-6-oxohexyl)-2-((E)-2-((E)-3-((E)-2-(3-(6-methoxy-6-oxohexyl)-1,1-dimethyl-1,3-dihydro-2H-benzo[e]indol-2-ylidene)ethylidene)cyclohex-1-en-1-yl) vinyl)-1,1-dimethyl-1H-benzo[e]indol-3-ium (BL-246-1): Prepared according to the general procedure from 2 (65 mg, 0.2 mmol, 1 equiv.) and BL-245 (167 mg, 0.4 mmol, 2 equiv.). Yield: 18% yield (30 mg). ¹H-NMR (600 μMHz, CD₃OD) § 8.23 (d, J=8.5 Hz, 2H), 8.01 (d, J=8.9 Hz, 2H), 7.99 (d, J=8.1 Hz, 2H), 7.87 (d, J=14.1 Hz, 2H), 7.65-7.62 (m, 2H), 7.57 (d, J=8.8 Hz, 2H), 7.51-7.46 (m, 3H), 6.23 (d, J=14.1 Hz, 2H), 4.27-4.19 (m, 4H), 3.69 (s, 3H), 3.62 (s, 3H), 2.62 (t, J=6.2 Hz, 4H), 2.36 (dt, J=11.5, 7.3 Hz, 4H), 2.06-1.96 (m, 12H), 1.93-1.88 (m, 4H), 1.76-1.70 (m, 4H), 1.56-1.50 (m, 4H). HRMS (ESI) calculated for [M, C₅₂H₆₁N₂O₄]⁺: 777.4626, found: 777.4626.

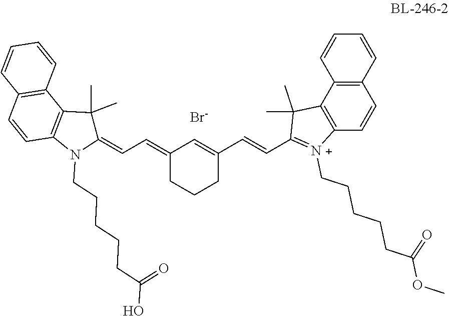 [SMILES: COC(=O)CCCCC[N+]1=C(/C=C/C2=CC(=C/C=C3/N(CCCCCC(=O)O)c4ccc5ccccc5c4C3(C)C)/CCC2)C(C)(C)c2c1ccc1ccccc21 ; [Br-]]

2-((E)-2-((E)-3-((E)-2-(3-(5-carboxypentyl)-1,1-dimethyl-1,3-dihydro-2H-benzo[e]indol-2-ylidene)ethylidene)cyclohex-1-en-1-yl) vinyl)-3-(6-methoxy-6-oxohexyl)-1,1-dimethyl-1H-benzo[e]indol-3-ium (BL-246-2): Prepared according to the general procedure from BL-85 (81 mg, 0.2 mmol, 1 equiv.)., 2 (65 mg, 0.2 mmol, 1 equiv.) and BL-245 (83 mg, 0.2 mmol, 1 equiv.). Yield: 17% yield (28 mg). ¹H-NMR (600 μMHz, CD₃OD) δ 8.23 (d, J=8.5 Hz, 2H), 8.21-7.96 (m, 4H), 7.87 (dd, J=14.1, 9.3 Hz, 2H), 7.65-7.61 (m, 2H), 7.59-7.54 (m, 2H), 7.51-7.45 (m, 3H), 6.22 (t, J=14.0 Hz, 2H), 4.27-4.18 (m, 4H), 3.70-3.61 (m, 3H), 2.61 (t, J=6.4 Hz, 4H), 2.36 (dt, J=11.5, 7.2 Hz, 2H), 2.29 (t, J=7.3 Hz, 2H), 2.03-2.00 (m, 8H), 1.97 (s, 6H), 1.94-1.87 (m, 4H), 1.76-1.68 (m, 4H), 1.57-1.50 (m, 4H); HRMS (ESI) calculated for [M, C₅₁H₅₉N₂O₄]⁺: 763.4469, found: 763.4455.

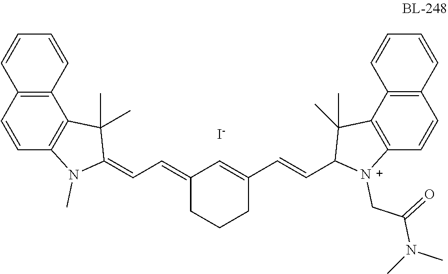 [SMILES: CN(C)C(=O)C[NH+]1c2ccc3ccccc3c2C(C)(C)C1/C=C/C1=CC(=C/C=C2/N(C)c3ccc4ccccc4c3C2(C)C)/CCC1 ; [I-]]

2-((E)-2-((E)-3-(2-(3-(3-(dimethylamino)-3-oxopropyl)-1,1-dimethyl-2,3-dihydro-1H-benzo[e]indol-2-yl)ethylidene)cyclohex-1-en-1-yl) vinyl)-1,1,3-trimethyl-1H-benzo[e]indol-3-ium (BL-248): Prepared according to the general procedure from BL-65 (70 mg, 0.2 mmol, 1 equiv.), 2 (65 mg, 0.2 mmol, 1 equiv.) and BL-65 (75 mg, 0.2 mmol, 1 equiv.). Yield: 20% yield (28 mg). ¹H-NMR (600 μMHz, CD₃OD) § 8.16-8.11 (m, 2H), 7.94-7.88 (m, 2H), 7.86-7.81 (m, 3H), 7.69 (d, J=13.8 Hz, 1H), 7.56-7.49 (m, 3H), 7.43-7.38 (m, 2H), 7.35-7.32 (m, 1H), 7.30 (d, J=8.8 Hz, 1H), 6.17 (d, J=14.3 Hz, 1H), 5.88 (d, J=13.7 Hz, 1H), 5.08 (s, 2H), 3.67 (s, 3H), 3.22 (s, 3H), 2.94 (s, 3H), 2.50 (t, J=6.2 Hz, 2H), 2.46 (t, J=6.2 Hz, 2H), 1.96 (s, 6H), 1.91 (s, 6H), 1.88-1.82 (m, 2H). ¹³C-NMR (151 μMHz, CD₃OD) δ 174.77, 172.68, 165.98, 154.83, 149.06, 146.27, 140.60, 140.25, 133.71, 132.79, 132.18, 132.13, 131.72, 130.32, 129.93, 129.74, 129.66, 128.19, 127.96, 127.38, 127.08, 124.76, 124.06, 122.02, 121.85, 110.61, 110.18, 100.14, 98.32, 51.01, 50.46, 44.88, 35.61, 34.87, 30.69, 26.28, 26.00, 23.59, 21.37. HRMS (ESI) calculated for [M, C₄₃H₄₆N₃O]⁺: 620.3635, found: 620.3634.

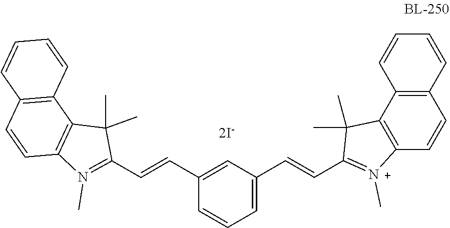

2,2′-((1E,1′E)-1,3-phenylenebis(ethene-2,1-diyl))bis(1,1,3-trimethyl-1H-benzo[e]indol-3-ium) (BL-250): Prepared according to the general procedure from m-Phthalaldehyde (70 mg, 0.52 mmol, 1 equiv.) and BL-68 (365 mg, 1.04 mmol, 2 equiv.). Yield: 50% yield (208 mg). ¹H-NMR (600 μMHz, CD₃OD) δ 8.64 (d, J=16.5 Hz, 2H), 8.48-8.44 (m, 2H), 8.34-8.30 (m, 2H), 8.27 (d, J=8.9 Hz, 2H), 8.19 (d, J=8.2 Hz, 2H), 8.04 (d, J=8.9 Hz, 2H), 7.96 (d, J=16.5 Hz, 2H), 7.87-7.83 (m, 2H), 7.81 (t, J=7.7 Hz, 2H), 7.76-7.73 (m, 2H), 4.45 (s, 6H), 2.18 (s, 12H). HRMS (ESI) calculated for [M, C₄₀H₃₈N₂]: 546.3024, found: 546.3015.

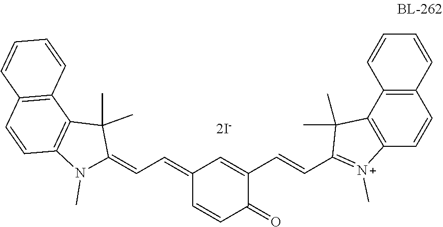

2,2′-((1E,1′E)-1,3-phenylenebis(ethene-2,1-diyl))bis(1,1,3-trimethyl-1H-benzo[e]indol-3-ium) (BL-262): Prepared according to the general procedure from 4-Hydroxyisophthalaldehyde (50 mg, 0.33 mmol, 1 equiv.) and BL-68 (232 mg, 0.66 mmol, 2 equiv.). Yield: 80% yield (182 mg). ¹H-NMR (600 μMHz, CD₃OD) δ 8.82 (s, 1H), 8.22 (d, J=16.0 Hz, 1H), 8.17 (d, J=8.5 Hz, 1H), 8.09 (d, J=8.8 Hz, 1H), 8.04 (d, J=8.1 Hz, 1H), 7.92 (d, J=8.6 Hz, 1H), 7.85 (d, J=16.0 Hz, 1H), 7.81 (dd, J=8.5, 1.3 Hz, 1H), 7.76 (d, J=8.5 Hz, 1H), 7.74-7.69 (m, 2H), 7.66-7.57 (m, 2H), 7.50 (d, J=10.3 Hz, 1H), 7.39-7.43 (m, 1H), 7.26-7.22 (m, 1H), 6.98 (d, J=8.6 Hz, 1H), 6.73 (d, J=8.4 Hz, 1H), 5.89 (d, J=10.3 Hz, 1H), 4.56 (s, 3H), 2.84 (s, 3H), 2.06 (s, 3H), 2.06 (s, 3H), 1.62 (s, 3H), 1.35 (s, 3H). HRMS (ESI) calculated for [M, C₄₀H₃₇N₂O]⁺: 561.2900, found: 561.2906.

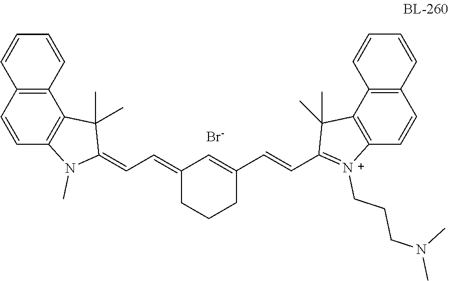 [SMILES: CN(C)CCC[N+]1=C(/C=C/C2=CC(=C/C=C3/N(C)c4ccc5ccccc5c4C3(C)C)/CCC2)C(C)(C)c2c1ccc1ccccc21 ; [Br-]]

2-((E)-2-((E)-3-(2-(3-(3-(dimethylamino) propyl)-1,1-dimethyl-2,3-dihydro-1H-benzo[e]indol-2-yl)ethylidene)cyclohex-1-en-1-yl) vinyl)-1,1,3-trimethyl-1H-benzo[e]indol-3-ium (BL-260): Prepared according to the general procedure from BL-68 (70 mg, 0.2 mmol, 1 equiv.), 2 (65 mg, 0.2 mmol, 1 equiv.) and BL-258 (91 mg, 0.2 mmol, 1 equiv.). Yield: 20% yield (28 mg). ¹H-NMR (600 μMHz, CD₃OD) δ 8.24 (d, J=9.1 Hz, 1H), 8.23 (d, J=9.1 Hz, 1H), 8.04-7.95 (m, 4H), 7.91 (d, J=14.2 Hz, 1H), 7.85 (d, J=13.9 Hz, 1H), 7.67-7.56 (m, 4H), 7.54-7.43 (m, 3H), 6.30-6.21 (m, 2H), 4.27 (t, J=7.2 Hz, 2H), 3.76 (s, 3H), 2.65-2.60 (m, 4H), 2.58-2.51 (m, 2H), 2.55 (s, 6H), 2.09-1.96 (m, 14H). HRMS (ESI) calculated for [M, C₄₄H₅₀N₃]⁺: 620.3999, found: 620.4000.

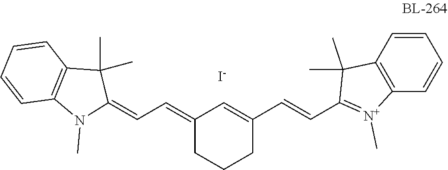 [SMILES: CN1/C(=C/C=C2C=C(/C=C/C3=[N+](C)c4ccccc4C3(C)C)CCC/2)C(C)(C)c2ccccc21 ; [I-]]

1,3,3-trimethyl-2-((E)-2-((E)-3-(2-((E)-1,3,3-trimethylindolin-2-ylidene)ethylidene)cyclohex-1-en-1-yl) vinyl)-3H-indol-1-ium (BL-264): Prepared according to the general procedure from BL-263 (60 mg, 0.2 mmol, 2 equiv.) and 2 (33 mg, 0.1 mmol, 1 equiv.). Yield: 87% yield (50 mg). ¹H-NMR (600 μMHz, CD₃OD) δ 7.76 (d, J=14.0 Hz, 2H), 7.49-7.44 (m, 3H), 7.39 (td, J=7.7, 1.2 Hz, 2H), 7.27-7.21 (m, 4H), 6.15 (d, J=14.0 Hz, 2H), 3.60 (s, 6H), 2.57 (t, J=6.2 Hz, 4H), 1.98-1.92 (m, 2H), 1.71 (s, 12H). ¹³C-NMR (151 μMHz, CD₃OD) δ 173.41, 149.81, 144.46, 142.27, 133.79, 129.68, 125.85, 123.24, 111.46, 100.77, 50.15, 31.40, 27.91, 24.96, 22.73. HRMS (ESI) calculated for [M, C₃₂H₃₇N₂]⁺: 449.2951, found: 449.2946.

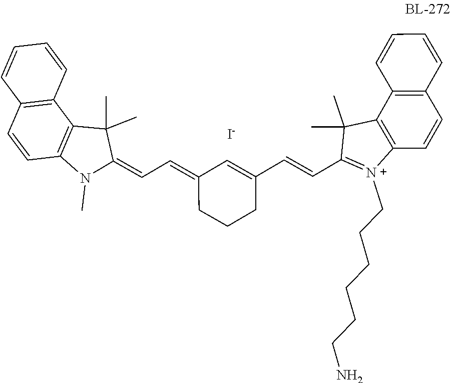 [SMILES: CN1/C(=C/C=C2C=C(/C=C/C3=[N+](CCCCCCN)c4ccc5ccccc5c4C3(C)C)CCC/2)C(C)(C)c2c1ccc1ccccc21 ; [I-]]

3-(6-aminohexyl)-1,1-dimethyl-2-((E)-2-((E)-3-((E)-2-(1,1,3-trimethyl-1,3-dihydro-2H-benzo[e]indol-2-ylidene)ethylidene)cyclohex-1-en-1-yl) vinyl)-1H-benzo[e]indol-3-ium (BL-272): Prepared according to the general procedure from BL-68 (70 mg, 0.2 mmol, 1 equiv.), 2 (65 mg, 0.2 mmol, 1 equiv.) and BL-116 (94 mg, 0.2 mmol, 1 equiv.). Yield: 10% yield (14 mg). ¹H-NMR (600 μMHz, CD₃OD) δ 8.24 (d, J=4.8 Hz, 1H), 8.22 (d, J=4.9 Hz, 1H), 8.03-7.95 (m, 4H), 7.90 (d, J=14.1 Hz, 1H), 7.85 (d, J=14.0 Hz, 1H), 7.65-7.61 (m, 2H), 7.58 (t, J=9.2 Hz, 2H), 7.54 (s, 1H), 7.49-7.44 (m, 2H), 6.25-6.17 (m, 2H), 4.25 (t, J=7.4 Hz, 2H), 3.74 (s, 3H), 2.95-2.90 (m, 2H), 2.64-2.58 (m, 4H), 2.04-1.96 (m, 14H), 1.94-1.88 (m, 2H), 1.72-1.66 (m, 2H), 1.59-1.48 (m, 4H). ¹³C-NMR (151 μMHz, CD₃OD) δ 175.21, 173.65, 156.10, 149.40, 148.36, 141.78, 141.29, 134.67, 134.44, 133.83, 133.51, 133.40, 133.22, 131.64, 131.11, 131.09, 129.99, 129.54, 129.42, 128.69, 128.68, 125.93, 125.85, 125.81, 123.35, 123.31, 118.00, 114.13, 111.97, 111.88, 100.80, 100.07, 52.13, 51.97, 44.80, 40.64, 31.90, 28.52, 28.40, 27.62, 27.56, 27.47, 27.29, 25.04, 22.80. HRMS (ESI) calculated for [M, C₄₅H₅₂N₃]⁺: 634.4156, found: 634.4153.

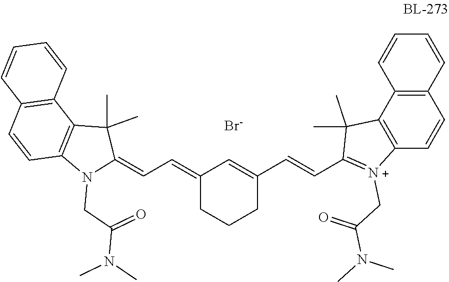 [SMILES: CN(C)C(=O)CN1/C(=C/C=C2C=C(/C=C/C3=[N+](CC(=O)N(C)C)c4ccc5ccccc5c4C3(C)C)CCC/2)C(C)(C)c2c1ccc1ccccc21 ; [Br-]]

3-(2-(dimethylamino)-2-oxoethyl)-2-((E)-2-((E)-3-((E)-2-(3-(2-(dimethylamino)-2-oxoethyl)-1,1-dimethyl-1,3-dihydro-2H-benzo[e]indol-2-ylidene)ethylidene)cyclohex-1-en-1-yl) vinyl)-1,1-dimethyl-1H-benzo[e]indol-3-ium (BL-273): Prepared according to the general procedure from 2 (33 mg, 0.1 mmol, 1 equiv.) and BL-247 (75 mg, 0.2 mmol, 2 equiv.). Yield: 26% yield (20 mg). ¹H-NMR (600 μMHz, CD₃OD) δ 8.24 (d, J=8.5 Hz, 2H), 7.98-7.94 (m, 4H), 7.87 (d, J=14.2 Hz, 2H), 7.65-7.61 (m, 2H), 7.51 (s, 1H), 7.47 (t, J=7.6 Hz, 2H), 7.44 (d, J=8.8 Hz, 2H), 6.06 (d, J=14.0 Hz, 2H), 5.24 (s, 4H), 3.32 (s, 6H), 3.04 (s, 6H), 2.55 (t, J=6.2 Hz, 4H), 2.06 (s, 12H), 1.96-1.90 (m, 2H). HRMS (ESI) calculated for [M, C₄₆H₅₁N₄O₂]⁺: 691.4007, found: 691.4002.

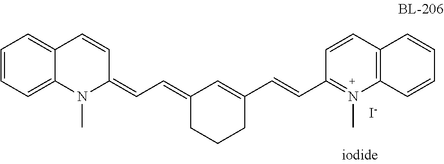

1-methyl-2-((E)-2-((E)-3-(2-((E)-1-methylquinolin-2 (1H)-ylidene)ethylidene)cyclohex-1-en-1-yl) vinyl) quinolin-1-ium (BL-206): Prepared according to the general procedure b from BL-69 (57 mg, 0.2 mmol, 2 equiv) and 2 (33 mg, 0.1 mmol, 1 equiv). Ochre solid. Yield: 70% (38 mg). ¹H-NMR (600 μMHz, DMSO-d6) δ 7.89 (s, 4H), 7.79-7.73 (m, 4H), 7.66-7.62 (m, 2H), 7.54 (d, J=13.6 Hz, 2H), 7.38-7.34 (m, 2H), 7.10 (s, 1H), 6.12 (d, J=13.6 Hz, 2H), 3.83 (s, 6H), 2.52-7.50 (m, 4H), 1.85-1.79 (m, 2H). ¹³C-NMR (151 μMHz, DMSO-d6) δ 150.63, 149.52, 143.90, 140.09, 135.16, 132.19, 130.46, 128.73, 124.64, 124.54, 120.05, 116.15, 104.06, 36.21, 24.24, 21.34. HRMS (ESI) calculated for [M, C₃₀H₂₉N₂]⁺: 417.2325, found: 417.2329.

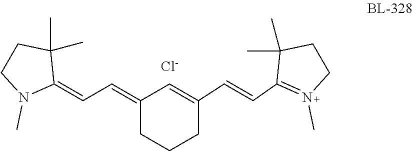

1,4,4-trimethyl-5-((E)-2-((E)-3-((E)-2-(1,3,3-trimethylpyrrolidin-2-ylidene)ethylidene)cyclohex-1-en-1-yl) vinyl)-3,4-dihydro-2H-pyrrol-1-ium (BL-328): Prepared according to the general procedure from BL-327 (50 mg, 0.3 mmol, 2 equiv) and BL-111 (50 mg, 0.15 mmol, 1 equiv). Yield: 50% (30 mg). ¹H-NMR (600 μMHz, CD₃OD) δ 7.49 (d, J=14.2 Hz, 2H), 7.08 (s, 1H), 5.52 (d, J=14.1 Hz, 2H), 3.70 (t, J=7.2 Hz, 4H), 3.15 (s, 6H), 2.40 (t, J=6.2 Hz, 4H), 2.00-1.94 (m, 4H), 1.88-1.82 (m, 2H), 1.47 (s, 12H).

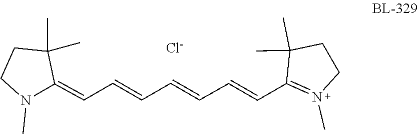

1,4,4-trimethyl-5-((1E,3E,5E,7E)-7-(1,3,3-trimethylpyrrolidin-2-ylidene) hepta-1,3,5-trien-1-yl)-3,4-dihydro-2H-pyrrol-1-ium (BL-329): Prepared according to the general procedure from BL-327 (50 mg, 0.3 mmol, 2 equiv) and Glutacondianil hydrochloride (44 mg, 0.15 mmol, 1 equiv). Yield: 50% (30 mg). ¹H-NMR (600 μMHz, CD₃OD) δ 7.68 (t, J=13.1 Hz, 2H), 7.27 (t, J=12.7 Hz, 1H), 6.19 (t, J=12.6 Hz, 2H), 5.64 (d, J=13.8 Hz, 2H), 3.69 (t, J=7.2 Hz, 4H), 3.14 (s, 6H), 2.00-1.92 (m, 4H), 1.46 (s, 12H).

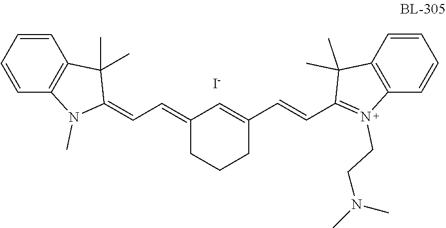

1-(2-(dimethylamino)ethyl)-3,3-dimethyl-2-((E)-2-((E)-3-(2-((E)-1,3,3-trimethylindolin-2-ylidene)ethylidene)cyclohex-1-en-1-yl) vinyl)-3H-indol-1-ium (BL-305): Prepared according to the general procedure from 2 (97 mg, 0.3 mmol, 1 equiv), BL-263 (90 mg, 0.3 mmol, 1 equiv) and BL-304 (117 mg, 0.3 mmol, 1 equiv). Yield: 26% yield (49 mg). ¹H-NMR (600 μMHz, CD₃OD) δ 7.84 (d, J=14.2 Hz, 1H), 7.73 (d, J=13.9 Hz, 1H), 7.54-7.49 (m, 2H), 7.48-7.43 (m, 2H), 7.43-7.38 (m, 1H), 7.34-7.32 (m, 1H), 7.31-7.27 (m, 1H), 7.25-7.20 (m, 2H), 6.25 (d, J=14.2 Hz, 1H), 6.18 (d, J=13.8 Hz, 1H), 4.20 (t, J=7.4 Hz, 2H), 3.67 (s, 3H), 2.72 (t, J=7.4 Hz, 2H), 2.63-2.57 (m, 4H), 2.42 (s, 6H), 1.99-1.94 (m, 2H), 1.74 (s, 6H), 1.73 (s, 6H).

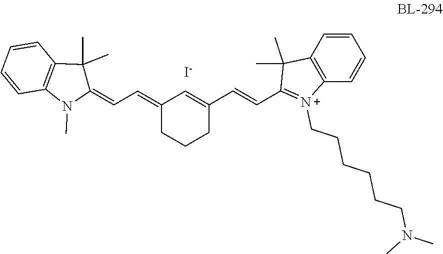 [SMILES: CN(C)CCCCCC[N+]1=C(/C=C/C2=CC(=C/C=C3/N(C)c4ccccc4C3(C)C)/CCC2)C(C)(C)c2ccccc21 ; [I-]]

1-(6-(dimethylamino) hexyl)-3,3-dimethyl-2-((E)-2-((E)-3-(2-((E)-1,3,3-trimethylindolin-2-ylidene)ethylidene)cyclohex-1-en-1-yl) vinyl)-3H-indol-1-ium (BL-294): Prepared according to the general procedure from BL-263 (90 mg, 0.3 mmol, 1 equiv), 2 (97 mg, 0.3 mmol, 1 equiv) and BL-293 (134 mg, 0.3 mmol, 1 equiv). Yield: 25% yield (52 mg). ¹H-NMR (600 μMHz, CD₃OD) δ 7.82-7.72 (m, 2H), 7.54 (s, 1H), 7.50-7.45 (m, 2H), 7.42-7.37 (m, 2H), 7.29-7.20 (m, 4H), 6.22-6.12 (m, 2H), 4.13 (t, J=7.4 Hz, 2H), 3.62 (s, 3H), 3.09-3.04 (m, 2H), 2.82 (s, 6H), 2.58 (t, J=6.2 Hz, 4H), 1.98-1.92 (m, 2H), 1.89-1.83 (m, 2H), 1.78-1.67 (m, 14H), 1.58-1.52 (m, 2H), 1.51-1.45 (m, 2H).

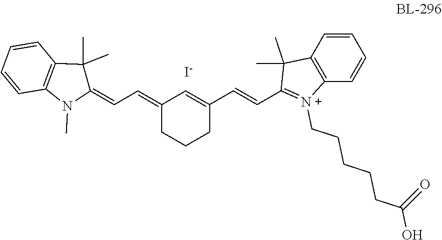 [SMILES: CN1/C(=C/C=C2C=C(/C=C/C3=[N+](CCCCCC(=O)O)c4ccccc4C3(C)C)CCC/2)C(C)(C)c2ccccc21 ; [I-]]

1-(5-carboxypentyl)-3,3-dimethyl-2-((E)-2-((E)-3-(2-((E)-1,3,3-trimethylindolin-2-ylidene)ethylidene)cyclohex-1-en-1-yl) vinyl)-3H-indol-1-ium (BL-296): Prepared according to the general procedure from 2 (65 mg, 0.2 mmol, 1 equiv), BL-263 (60 mg, 0.2 mmol, 1 equiv) and BL-295 (71 mg, 0.2 mmol, 1 equiv). Yield: 30% yield (41 mg). ¹H-NMR (600 μMHz, CD₃OD) δ 7.83-7.69 (m, 2H), 7.55-7.43 (m, 3H), 7.42-7.36 (m, 2H), 7.29-7.18 (m, 4H), 6.24-6.08 (m, 2H), 4.15-4.06 (m, 2H), 3.69-3.54 (m, 3H), 2.68-2.45 (m, 4H), 2.30 (t, J=7.3 Hz, 2H), 1.98-1.91 (m, 2H), 1.86-1.81 (m, 2H), 1.78-1.60 (m, 14H), 1.53-1.48 (m, 2H).

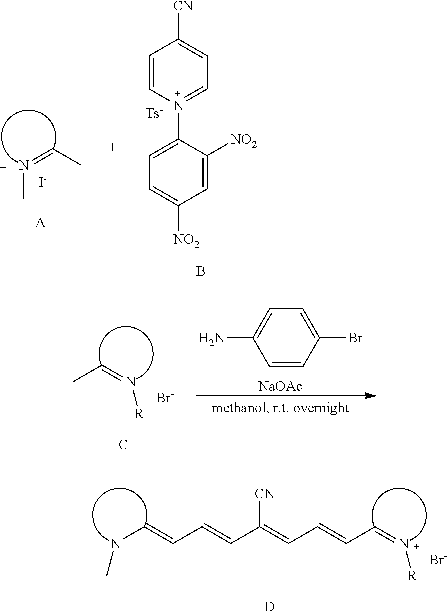

General procedure: The corresponding pyridinium salt B (1 equiv.) and 4-bromoaniline (1 equiv.) were dissolved in methanol (4 mL) in an 8 mL vial, and the mixture was stirred at room temperature for 30 min. Next, a heterocyclic salt A (1 equiv.), C (1 equiv.) and sodium acetate (3 equiv.) were added. The reaction mixture was stirred for an additional overnight at room temperature. The crude product D was purified by flash column chromatography (silica gel, dichloromethane/methanol).

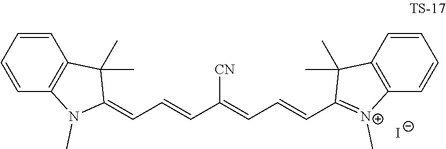 [SMILES: CN1/C(=C/C=C/C(C#N)=C/C=C/C2=[N+](C)c3ccccc3C2(C)C)C(C)(C)c2ccccc21 ; [I-]]

2-((1E,3Z,5E)-4-cyano-7-((E)-1,3,3-trimethylindolin-2-ylidene)-hepta-1,3,5-trien-1-yl)-1,3,3-trimethyl-3H-indol-1-ium iodide: TS-17 was afforded as a green solid in 90% yield. 1H NMR (600 μMHz, Methanol-d4) δ 7.96 (t, J=16.2 Hz, 2H), 7.41 (td, J=9.6, 1.8 Hz, 2H), 7.39 (d, J=9.0 Hz, 2H), 7.28 (t, J=9.0 Hz, 2H), 7.17 (d, J=9.0 Hz, 2H), 6.88 (bro, 2H), 3.78 (s, 6H), 1.71 (s, 12H).

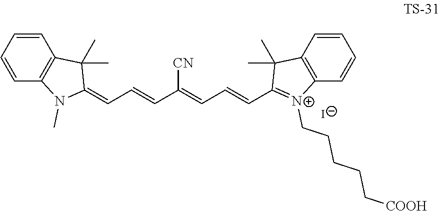 [SMILES: CN1/C(=C/C=C/C(C#N)=C/C=C/C2=[N+](CCCCCC(=O)O)c3ccccc3C2(C)C)C(C)(C)c2ccccc21 ; [I-]]

1-(5-Carboxypentyl)-2-((1E,3Z,5E)-4-cyano-7-((E)-1,3,3-trimethylindolin-2-ylidene)-hepta-1,3,5-trien-1-yl)-3,3-dimethyl-3H-indol-1-ium iodide: TS-31 was afforded as a green solid in 41% yield. ¹H NMR (600 μMHz, Methanol-d4) δ 8.09 (td, J=15.6, 7.8 Hz, 2H), 7.59-7.56 (m, 2H), 7.50-7.46 (m, 2H), 7.41 (t, J=10.2 Hz, 2H), 7.38-7.33 (m, 2H), 6.83 (dd, J=15.0, 4.8 Hz, 2H), 6.57 (dd, J=16.8, 9.6 Hz, 2H), 4.20 (t, J=9.0 Hz, 2H), 3.73 (s, 3H), 2.33 (t, J=9.0 Hz, 2H), 1.89-1.86 (m, 2H), 1.75 (d, t, J=4.2 Hz. 12H), 1.73-1.68 (m, 2H), 1.56-1.51 (m, 2H).

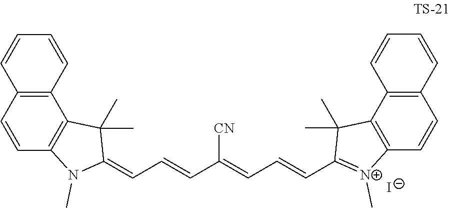 [SMILES: CN1/C(=C/C=C/C(C#N)=C/C=C/C2=[N+](C)c3ccc4ccccc4c3C2(C)C)C(C)(C)c2c1ccc1ccccc21 ; [I-]]

2-((1E,3Z,5E,7E)-4-cyano-7-(1,1,3-trimethyl-1,3-dihydro-2H-benzo[e]indol-2-ylidene)-hepta-1,3,5-trien-1-yl)-1,1,3-trimethyl-1H-benzo[e]indol-3-ium iodide: TS-21 was afforded as a green solid in 78% yield. 1H NMR (600 μMHz, Methanol-d4) δ 8.29 (d, J=10.2 Hz, 2H), 8.16 (t, J=16.2 Hz, 2H), 8.07 (d, J=, 10.8 Hz, 2H), 8.03 (d, J=10.2 Hz, 2H), 7.67 (t, J=10.8 Hz, 4H), 7.53 (t, J=9.0 Hz, 2H), 6.83 (d, J=9.0 Hz, 2H), 6.58 (d, J=16.2 Hz, 2H), 6.57 (dd, J=16.8, 9.6 Hz, 2H), 3.82 (s, 6H), 2.02 (d, J=25.2 Hz, 12H).

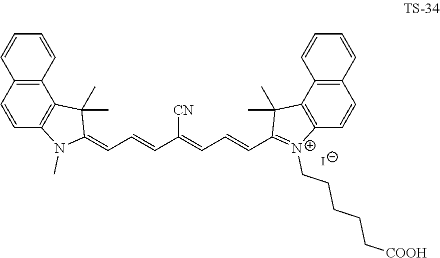 [SMILES: CN1/C(=C/C=C/C(C#N)=C/C=C/C2=[N+](CCCCCC(=O)O)c3ccc4ccccc4c3C2(C)C)C(C)(C)c2c1ccc1ccccc21 ; [I-]]

3-(5-Carboxypentyl)-2-((1E,3Z,5E,7E)-4-cyano-7-(1,1,3-trimethyl-1,3-dihydro-2H-benzo[e]indol-2-ylidene)-hepta-1,3,5-trien-1-yl)-1,1-dimethyl-1H-benzo[e]indol-3-ium iodide: TS-34 was afforded as a green solid in 34% yield. 1H NMR (600 μMHz, Methanol-d4) δ 8.29 (dd, J=9.6, 4.2 Hz, 2H), 8.17 (d, J=8.4 Hz, 2H), 8.06 (dd, J=10.8, 6.0 Hz, 2H), 8.03 (dd, J=10.2, 3.6 Hz, 2H), 7.69-7.65 (m 4H), 7.55-7.51 (m, 2H), 6.85 (dd, J=15.6, 8.4 Hz, 2H), 6.60 (dd, J=16.8, 6.0 Hz, 2H), 4.30 (t, J=9.0 Hz, 2H), 2.31 (t, J=9.0 Hz, 2H), 2.02 (d, t, J=1.2 Hz, 12H), 1.94-1.89 (m, 2H), 1.73-1.69 (m, 2H), 1.59-1.54 (m, 2H).

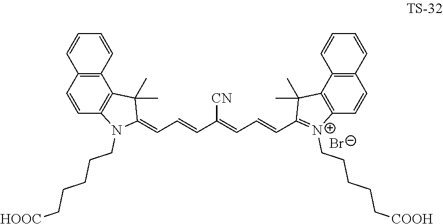

6-(2-((1E,3Z,5E,7E)-7-(3-(5-Carboxypentyl)-1,1-dimethyl-1,3-dihydro-2H-benzo[e]indol-2-ylidene)-4-cyanohepta-1,3,5-trien-1-yl)-1,1-dimethyl-1H-314-benzo[e]indol-3-yl)-hexanoic acid bromide: TS-32 was afforded as a green solid in 42% yield. 1H NMR (600 μMHz, Methanol-d4) δ 7.93 (bro, 2H), 7.44 (d, J=7.2 Hz, 2H), 7.36-7.33 (m, 2H), 7.30 (d, J=7.8 Hz, 2H), 7.21 (t, J=7.2 Hz, 2H), 6.73 (d, J=13.2 Hz, 2H), 6.47 (d, J=13.8 Hz, 2H), 4.07 (t, J=7.8 Hz, 4H), 2.09 (t, J=7.8 Hz, 4H), 1.76-1.73 (m, 4H), 1.61 (s, 12H), 1.60-156 (, 4H), 1.41-1.38 (m, 4H).

### Example 2—Charged Pi-Conjugated Compounds as Narrow-Spectrum Antibacterial Agents Against Antibiotic-Resistant Gram-Positive Pathogens

The data presented below provides evidence that charged pi-conjugated compounds have potent bacteriostatic and bactericidal activity against various Gram-positive pathogens, including antibiotic-resistant strains of Staphylococcus aureus and Enterococcus faecalis, even in the absence of light. In addition to exponential phase cells, charged pi-conjugated compounds were effective in eradicating antibiotic-tolerant phenotypes, such as bacterial persisters and the established biofilms of some Gram-positive strains, performing better than conventional antibiotics. In contrast, charged pi-conjugated compounds of the present disclosure showed no significant inhibitory effect on Gram-negative bacteria. Repeated exposure to charged pi-conjugated compounds is shown according to the data presented below to result in low levels of resistance development compared with standard antibiotics. Mechanistic studies revealed, without being bound by any particular theory, that charged pi-conjugated compounds disrupted bacterial membrane-associated metabolic processes, particularly energy metabolism, as revealed by the observation of charged pi-conjugated compound-induced changes in the expression of genes related to ATP synthesis and membrane transport. Charged pi-conjugated compounds were also found, again without being bound by any particular theory, to act synergistically with conventional antibiotics, denoting their distinctive mechanism of antibacterial action. Biocompatibility evaluations revealed dose-dependent cytotoxicity in mammalian cell lines. Preliminary in vivo assessments in Galleria mellonella showed protective effects of charged pi-conjugated compounds at low doses on worm survival and promising therapeutic potential against MRSA infections. For at least these reasons, the presently disclosed charged pi-conjugated compounds represent a promising new class of narrow-spectrum antibacterial agents to combat antibiotic-resistant Gram-positive pathogens.

### a. Evaluation of Antibacterial Properties of Charged Pi-Conjugated Compounds

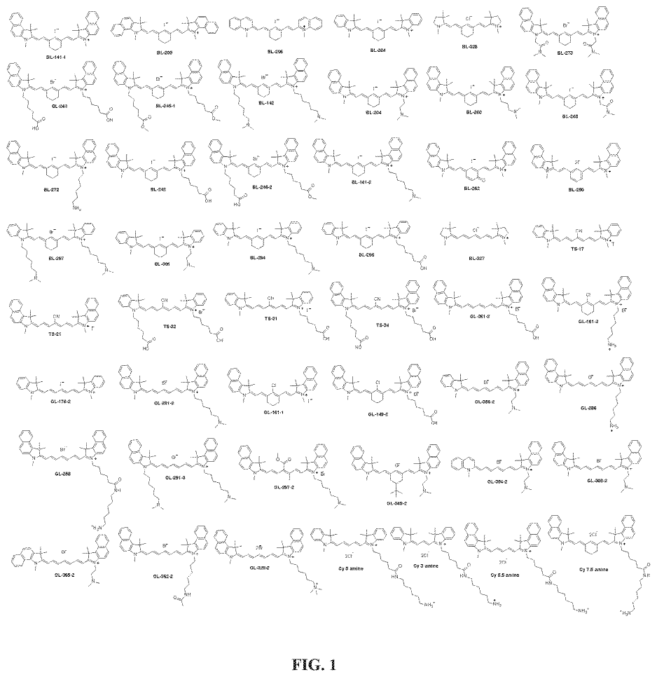

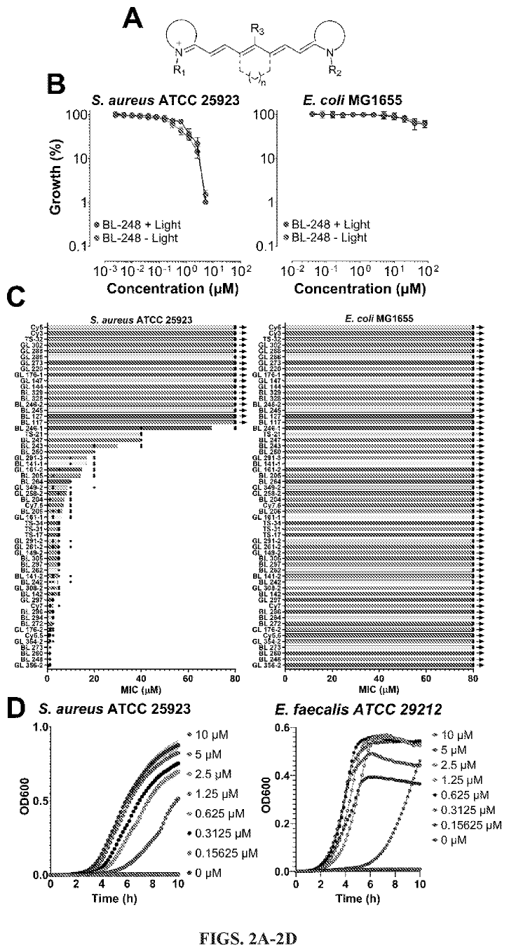

A library of 56 compounds with various amine groups at distinct regions of the molecule was constructed (FIG. 1, FIG. 2A). Different amine groups were incorporated into the molecules to enhance their affinity for the negatively charged bacterial membrane because amine groups, without being bound, usually acquire a positive charge under physiological conditions.

For the initial tests, two bacterial strains were used: S. aureus was used as a representative Gram-positive bacterium, and E. coli was used as a representative Gram-negative bacterium. Bacterial cell suspensions were prepared and subsequently treated with increasing concentrations (16 to 80 μM) of a representative charged pi-conjugated compound. Charged pi-conjugated compound-treated cell suspensions were then exposed to near-infrared radiation (NIR) at 80 mW cm⁻² for 10 min, equivalent to a fluence of 48 J cm⁻² (UHP-F-730; Prizmatix, Israel). DMSO-treated controls were included to assess the potential impact of the solvent. After irradiation, cell suspensions were collected, inoculated into cation-adjusted Mueller-Hinton broth (CA-MHB), and then incubated overnight at 37° C. The samples were then evaluated for growth. The minimum inhibitory concentration (MIC) was determined as the concentration of compound at which no discernible bacterial growth was evident.

FIG. 2B shows the concentration-dependent growth inhibition patterns of E. coli and S. aureus treated with a representative charged pi-conjugated compound. For S. aureus, increasing concentrations of charged pi-conjugated compound gradually reduced bacterial growth, which was consistent for both the irradiated and non-irradiated samples. Interestingly, in the case of E. coli, treatment with charged pi-conjugated compounds resulted in a maximum reduction of ~40% in bacterial growth, even at the highest concentration tested. This trend was consistent for both the irradiated and non-irradiated samples.

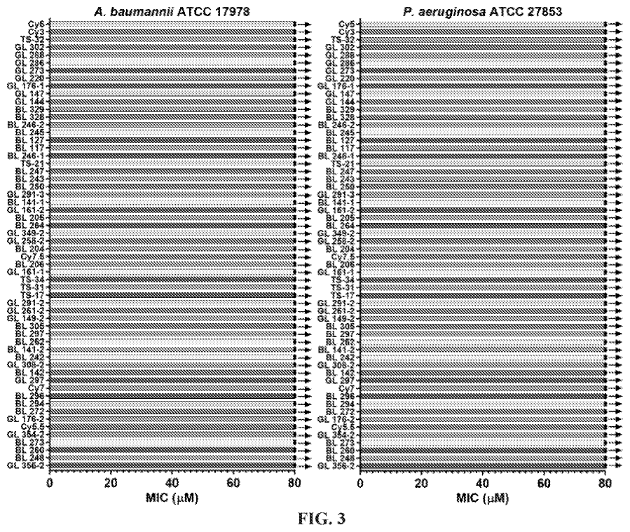

Building on these initial observations, the antibacterial potential of the entire library of 56 charged pi-conjugated compounds was further explored in both E. coli and S. aureus in the absence of light. The MICs of different charged pi-conjugated compounds in E. coli and S. aureus are shown in FIG. 2C. Several of the molecules displayed potent bacteriostatic properties against the Gram-positive bacterium S. aureus, with average MIC values fluctuating between 1.1 μM and ≥80 μM (FIG. 2C). For the Gram-negative bacterium E. coli, every molecule displayed MIC values higher than the highest concentration tested. Similar results were obtained with two other Gram-negative bacteria, Pseudomonas aeruginosa and Acinetobacter baumannii (FIG. 3).

The bacteriostatic activity of charged pi-conjugated compounds was further investigated by monitoring the growth curves of S. aureus and E. faecalis upon treatment with increasing concentrations of representative charged pi-conjugated compounds (FIG. 2D). With increasing charged pi-conjugated compound concentration, a marked reduction in bacterial growth was observed, characterized by a decrease in the maximum OD₆₀₀ and an extended lag phase. At the highest concentration tested (10 μM), bacterial growth was completely inhibited, indicating a clear bacteriostatic and/or bactericidal effect.

These results suggest, without being bound by theory, that charged pi-conjugated compounds are narrow-spectrum antibacterial agents that are specific to Gram-positive bacteria. Gram-negative bacteria have an inner and an outer membrane enclosing a thin peptidoglycan layer. In contrast, Gram-positive bacteria have a dense peptidoglycan cell wall, but no outer membrane (Silhavy et al., 2010). The increased permeability of the Gram-positive cell wall probably, again without being bound by theory, allows more efficient uptake of the positively charged pi-conjugated compounds, which explains their efficacy against Gram-positive bacteria such as S. aureus but not against Gram-negative species.

Significantly, charged pi-conjugated compounds exhibited antibacterial activity independent of the presence of light, which distinguishes them from previously described photosensitizing cyanine derivatives used in photodynamic therapy (PDT). In PDT, light-absorbing molecules called photosensitizers (such as some cyanine derivatives) are excited by light of a specific wavelength, producing reactive oxygen species (ROS) such as singlet oxygen, which can cause cell damage or death (Lange et al., 2021). Accordingly, a recent study reported a new series of cyclohexene-based cyanine dyes containing two and three indolenine, benzothiazole, and benzoselenazole terminal end groups that were highly efficient near-IR photosensitizers for antibacterial PDT (aPDT) (Prakash et al., 2023). However, aPDT also has several limitations (Niculescu & Grumezescu, 2011). A major limitation is the depth of light penetration. Effective aPDT requires that the photosensitizer, oxygen, and light interact in the target area. If bacteria are situated deep within tissues, it might be challenging for light of the required wavelength to reach them, especially when using visible light. Near-infrared (NIR) light can penetrate deeper but is not absorbed by all photosensitizers. Another significant limitation is that aPDT depends on the presence of molecular oxygen to produce reactive oxygen species (ROS) that mediate bacterial killing. Thus, the effectiveness of aPDT can be compromised in hypoxic or anaerobic environments. Because the presently disclosed charged pi-conjugated compounds do not require light to exert their antibacterial action, they circumvent the restrictions associated with inadequate light penetration and oxygen dependence that constrain aPDT applications.

The investigation of the bacteriostatic potential of compounds of the present disclosure was expanded to a wide range of Gram-positive bacteria of various genera, specifically Aerococcus, Bacillus, Corynebacterium, Enterococcus, Listeria, Micrococcus, Propionibacterium, Rothia, Staphylococcus, and Streptococcus. In addition, the antibacterial effects of charged pi-conjugated compounds in Mycobacterium smegmatis, a mycobacterial species, was also evaluated. Mycobacteria are characterized by a unique cell wall structure with a thick peptidoglycan layer and high mycolic acid concentration and, as a result, cannot be stained using conventional Gram staining techniques. They are categorized using alternative methods, such as Ziehl-Neelsen or acid-fast staining. Despite this resistance, mycobacteria are occasionally associated with Gram-positive bacteria because of their shared thick peptidoglycan layer (Maitra et al., 2019).

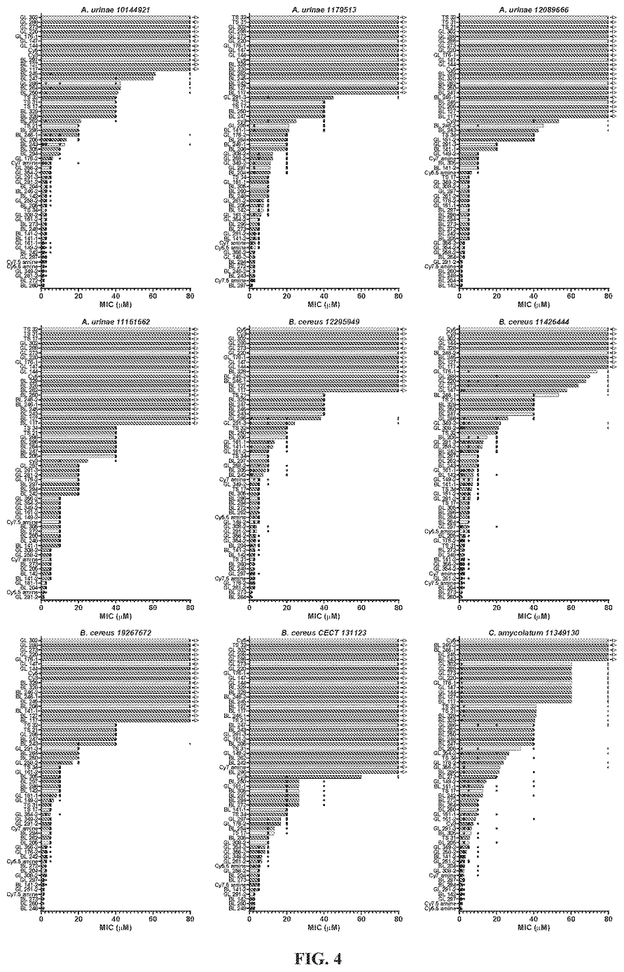

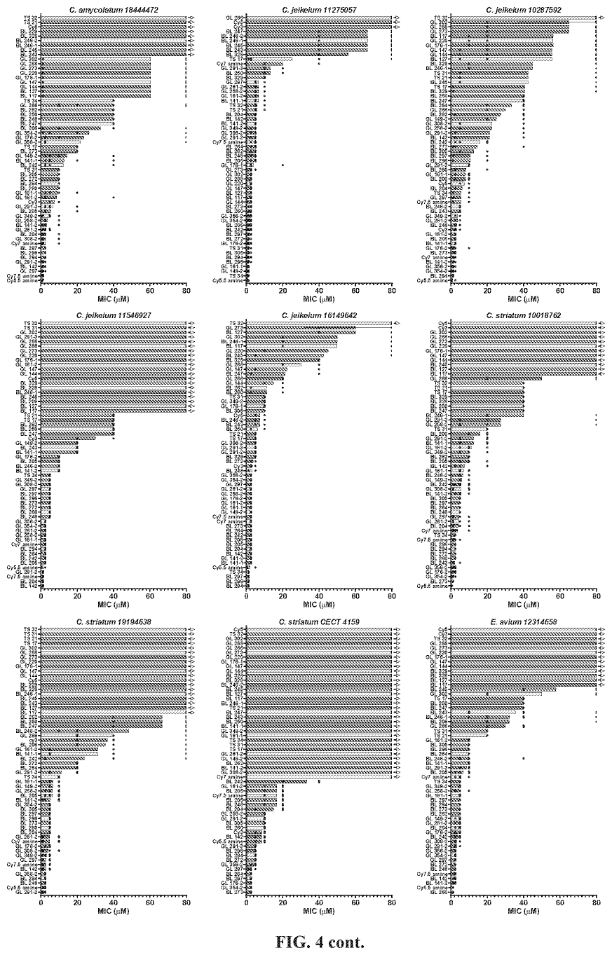

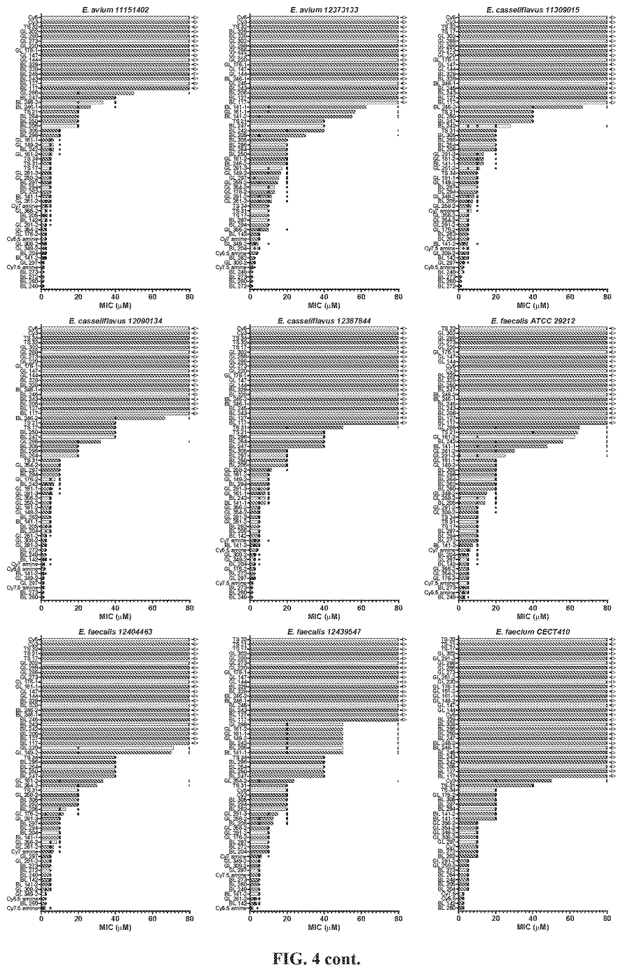

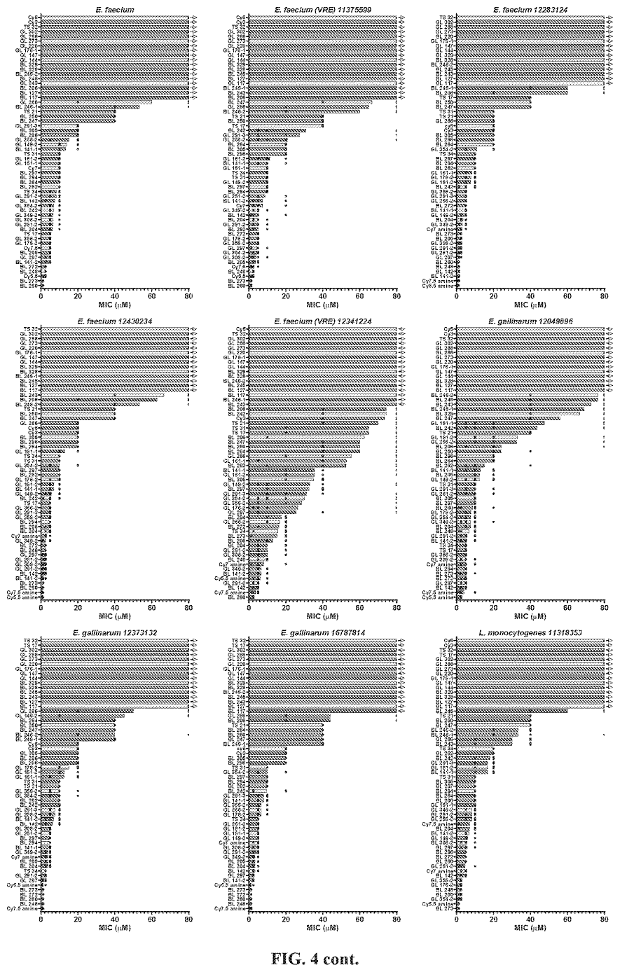

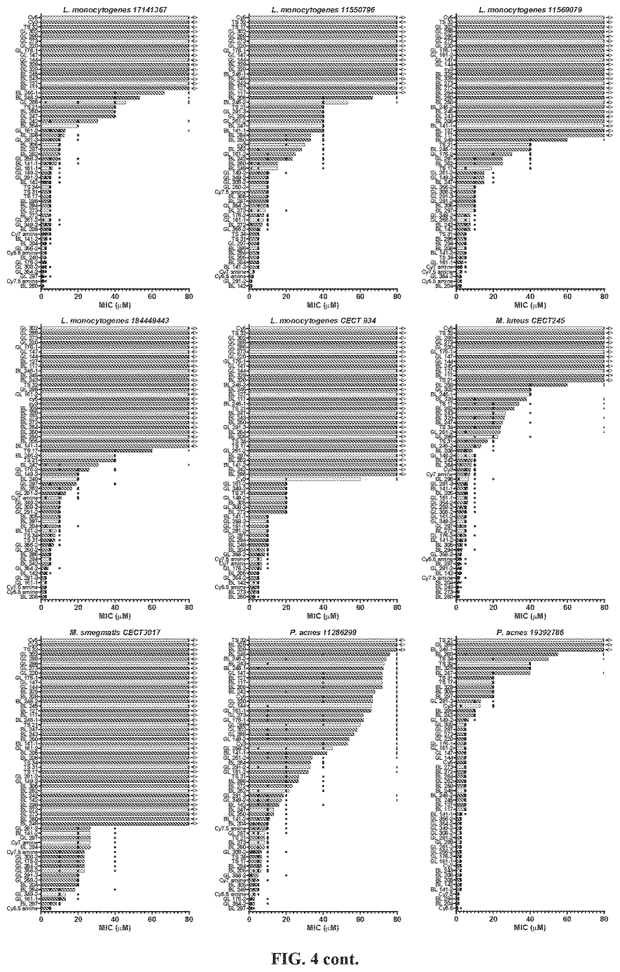

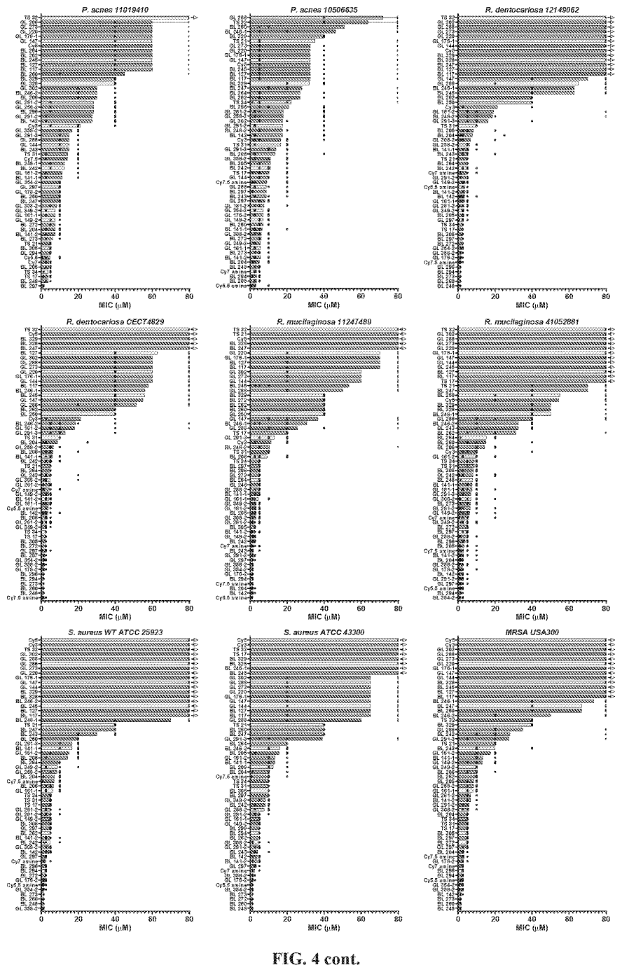

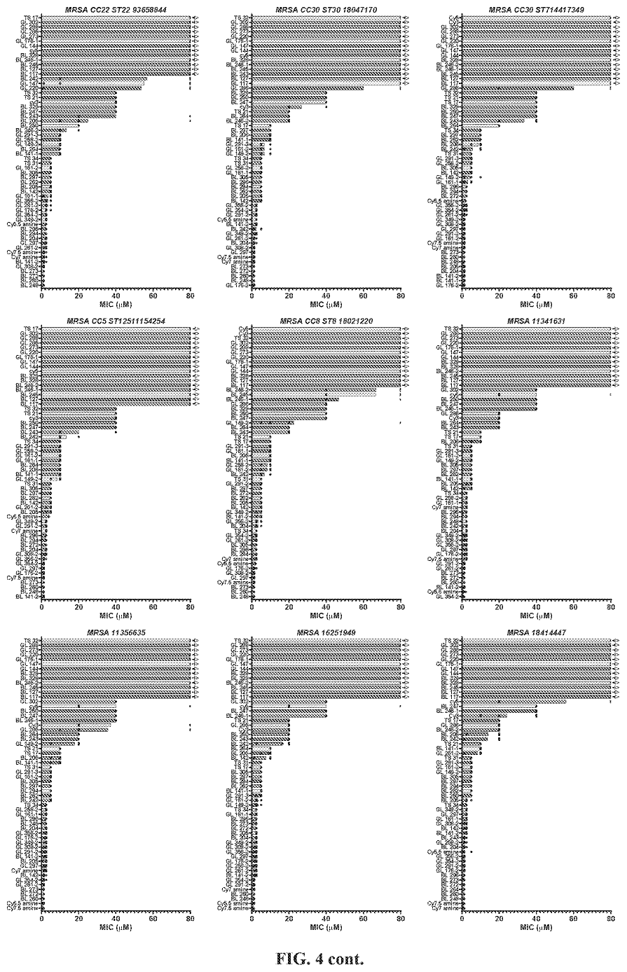

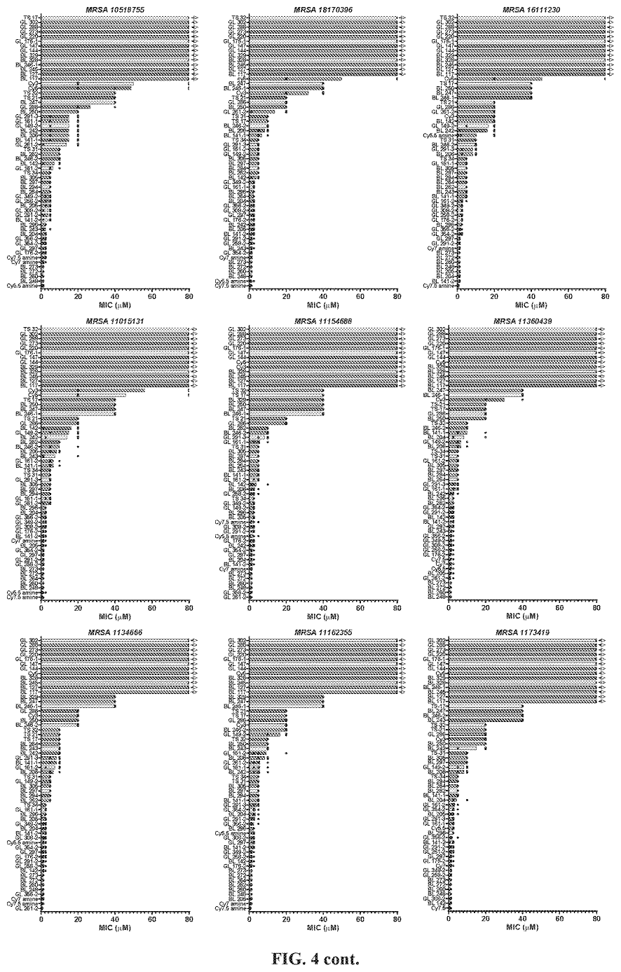

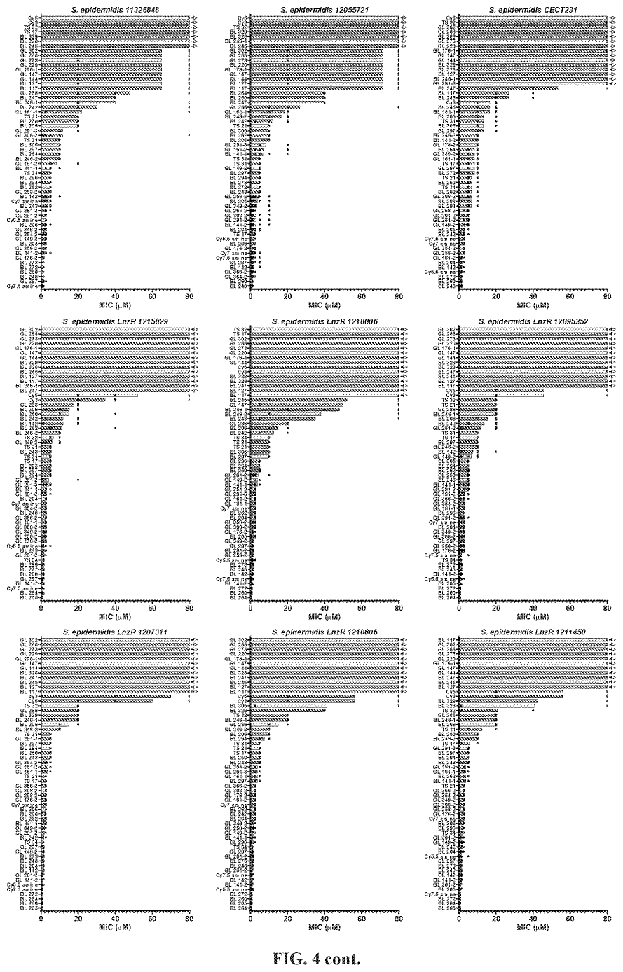

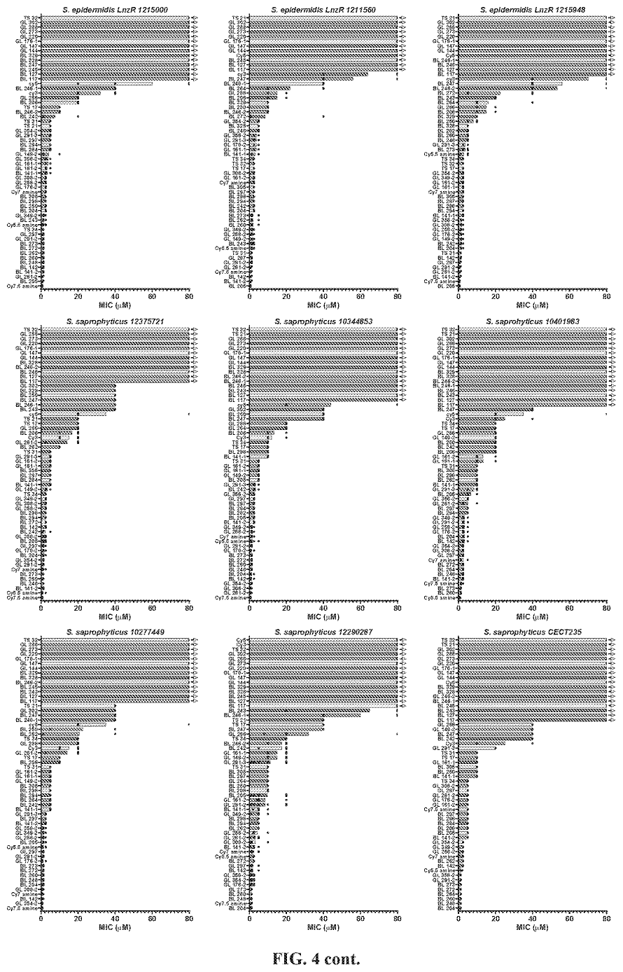

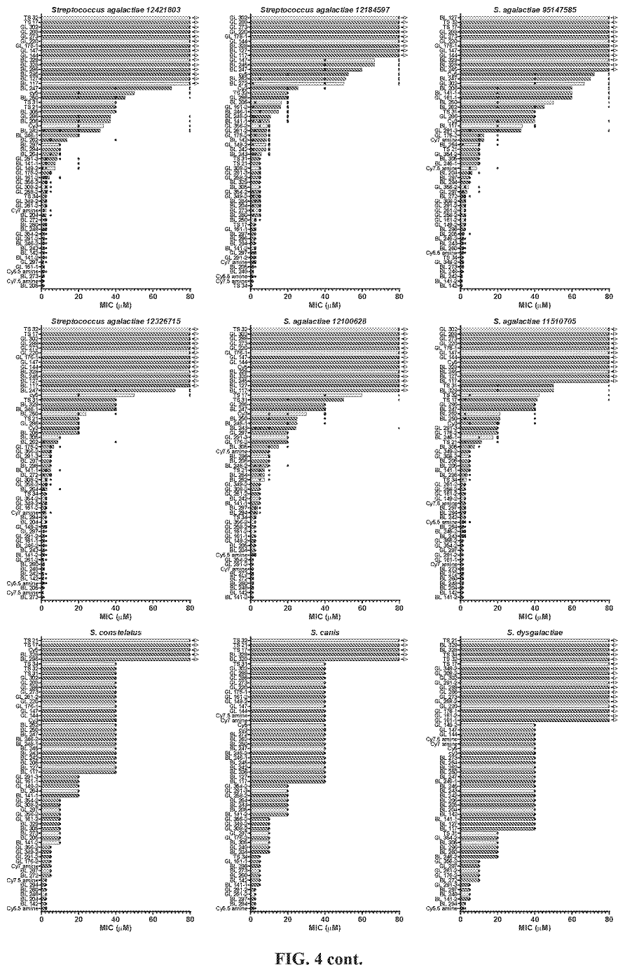

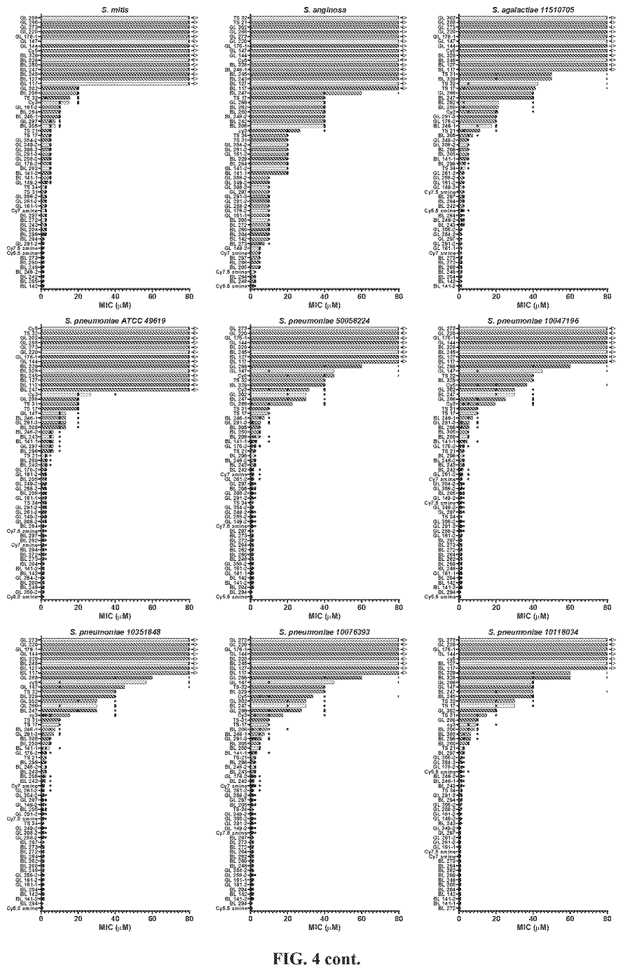

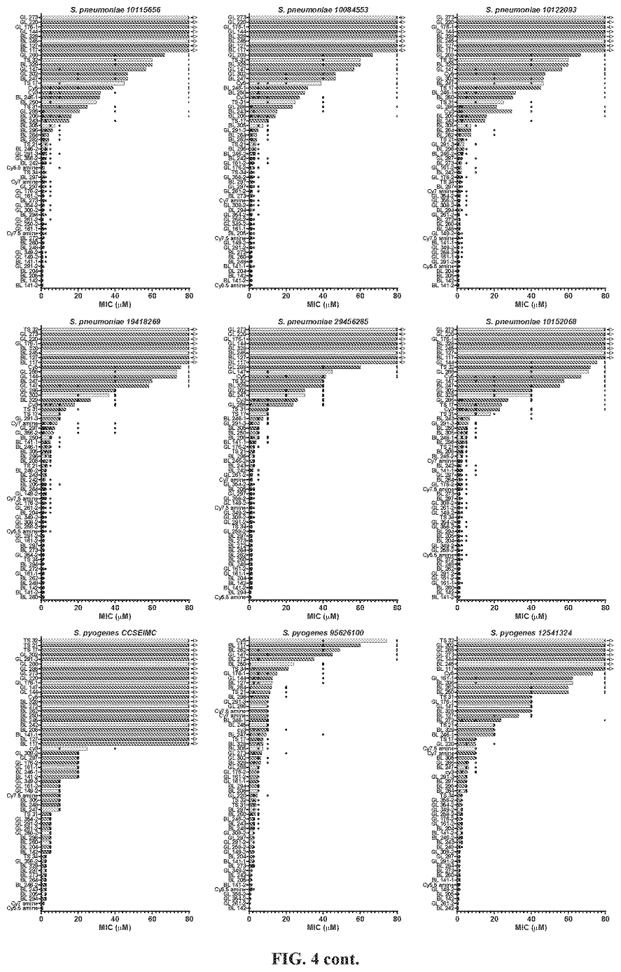

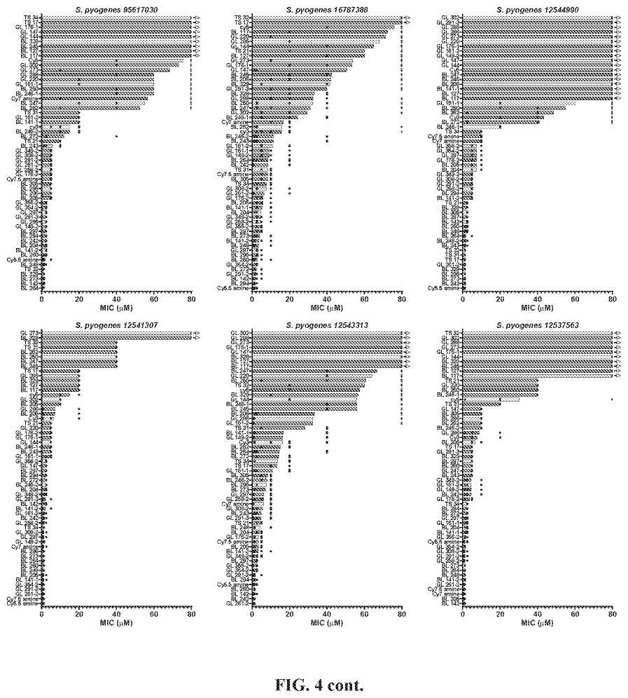

Antibacterial activity was assessed by determining the minimal inhibitory concentration (MIC) and the minimal bactericidal concentrations (MBC) and by monitoring time-dependent bacterial killing of exponentially growing cells. Bacterial cell suspensions (OD₆₀₀≈0.02 in cation-adjusted Muller-Hinton Broth) were treated with increasing concentrations of the different molecules. The minimal inhibitory concentration (MIC) was defined as the minimal concentration of the molecule resulting in complete bacterial growth arrest. The minimal bactericidal concentration (MBC) was defined as the lowest concentration of the molecule that killed 99.9% of the inoculum. A molecule was determined to be bactericidal if the ratio of the MBC to the MIC was less than 4 to 6. The MIC values of various charged pi-conjugated compounds against different representative strains are shown in FIG. 4. Several molecules showed potent antibacterial activity against all the Gram-positive strains tested, with average MIC values ranging from 0.88 μM to ≥80 μM. In M. smegmatis, the antibacterial activity of charged pi-conjugated compounds was generally lower. Nevertheless, some molecules showed significant antibacterial activity, with MIC values ranging from 5 μM to ≥80 μM.

The differential activity of charged pi-conjugated compounds against Gram-positive bacteria and Mycobacterium smegmatis can be attributed, without being bound by theory, to the structural and compositional differences in their cell walls. The cell wall of Gram-positive bacteria mainly consists of a relatively porous, thick peptidoglycan layer, which may allow large molecules such as charged pi-conjugated compounds to easily penetrate and exert their action. The cell wall of mycobacteria, including Mycobacterium smegmatis, is unique. While it contains a thick peptidoglycan layer similar to that of Gram-positive bacteria, it also contains high concentrations of mycolic acids. These mycolic acids form a hydrophobic barrier that can be a formidable obstacle for many drugs, reducing their penetration and efficacy (Batt et al., 2020). Moreover, positively charged pi-conjugated compounds are expected, without being bound by theory, to have a higher affinity for negatively charged bacterial membranes. While the Gram-positive cell wall contains teichoic acids that are negatively charged, the mycobacterial cell wall, with its complex layer of mycolic acids, glycolipids, and other lipids, might not provide, again without being bound by theory, the same degree of charge attraction for compounds of the present disclosure (Maitra et al., 2019).

Importantly, charged pi-conjugated compounds showed robust bacteriostatic effects against antibiotic-resistant strains such as methicillin-resistant S. aureus (MRSA), linezolid-resistant S. epidermidis, and vancomycin-resistant enterococci (VRE) (FIG. 4).

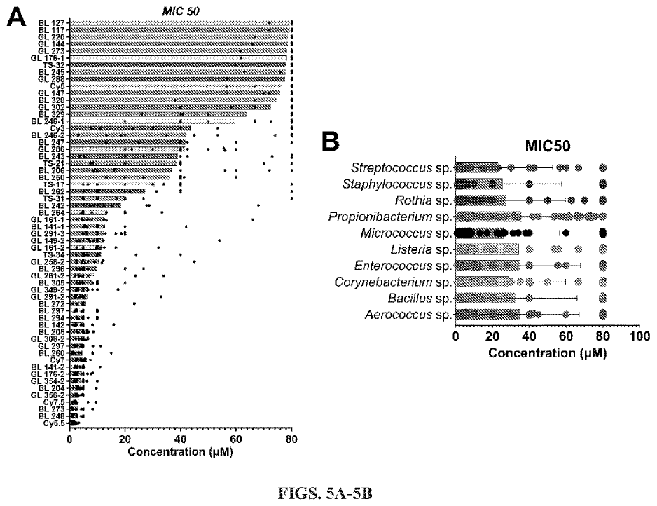

The MIC values for all Gram-positive strains tested (Table 2) were used to calculate the MIC₅₀, i.e., the minimum inhibitory concentration needed to inhibit the growth of 50% of the tested isolates (FIG. 5A). According to the MIC₅₀ results, the molecules with the strongest antibacterial activity were Cy5.5 amine, BL 248, BL 273, Cy7.5 amine, and GL 356-2. When the average MIC₅₀ values were evaluated, the most susceptible bacterial genera were Streptococcus sp., followed by Staphylococcus sp. In contrast, Propionibacterium sp. had the highest average MIC₅₀ (FIG. 5B).

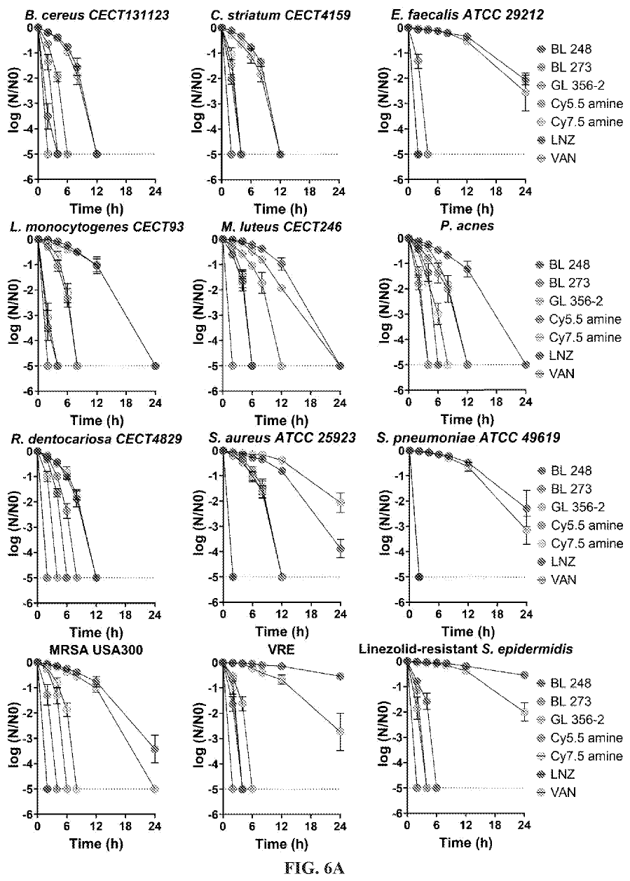

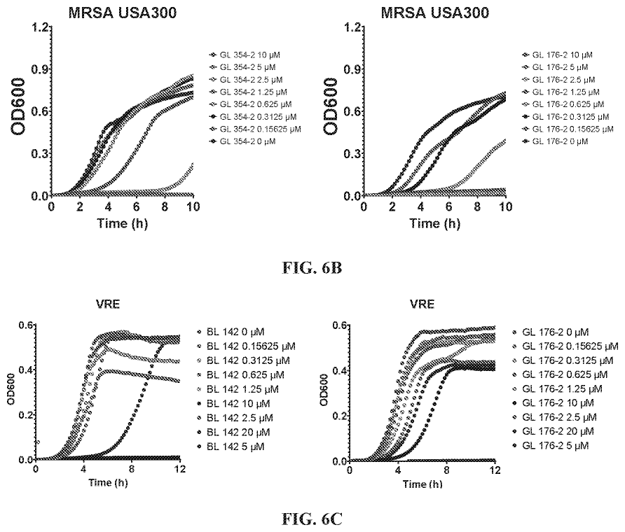

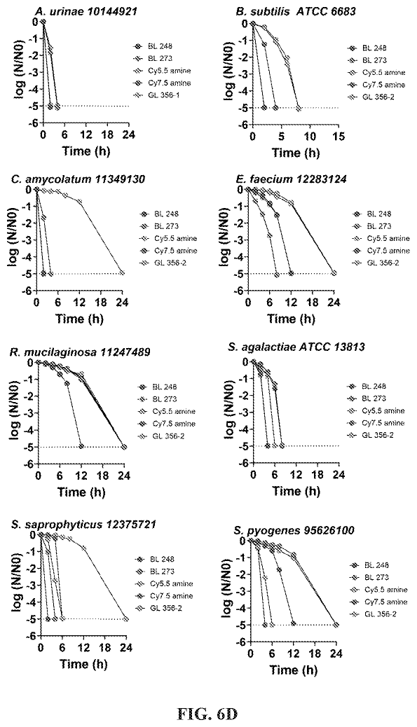

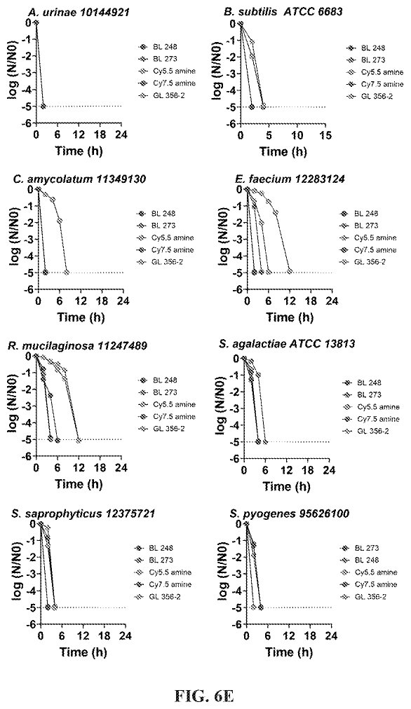

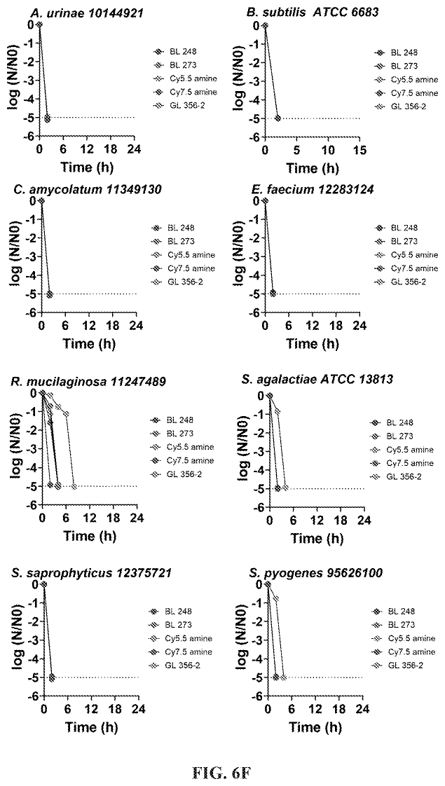

Time-kill experiments were performed to evaluate the bactericidal activity of the most potent charged pi-conjugated compounds identified from the calculation of MIC₅₀ values. Cell suspensions of various representative Gram-positive strains in their exponential phase were exposed to different charged pi-conjugated compounds for increasing time periods. The conventional antibiotics linezolid and vancomycin were used as controls. Treatment with charged pi-conjugated compounds effectively eradicated bacterial populations (i.e., ~5 log 10 reduction), including antibiotic-resistant strains, within 2-12 h, outpacing the efficacy of conventional antibiotics for all strains tested (FIG. 6).

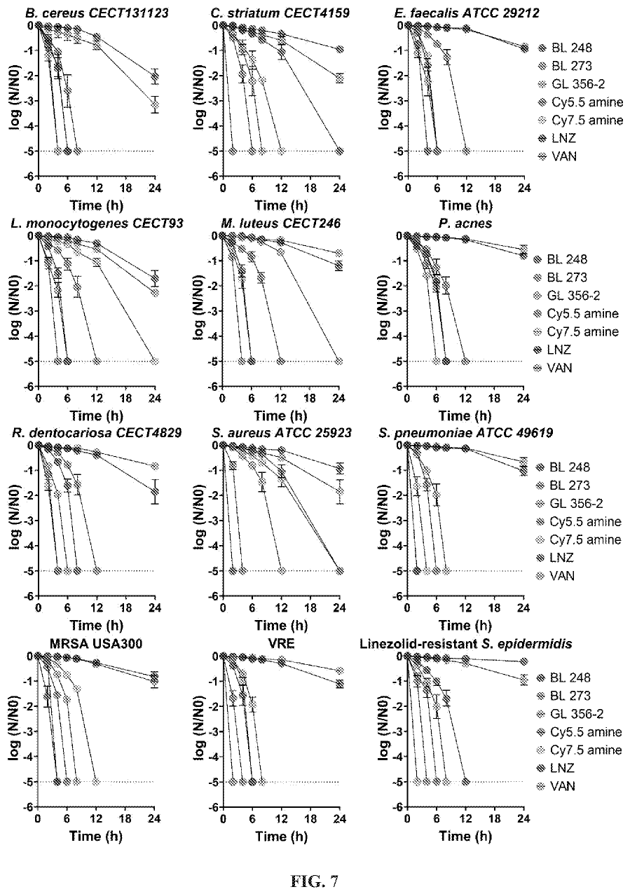

The potential of charged pi-conjugated compounds to eradicate bacterial persister cells was also investigated. Persister cells are a small subset of dormant and highly antibiotic-tolerant phenotypic variants present in bacterial populations that are genetically indistinguishable from wild-type populations (Huemer et al., 2020). Charged pi-conjugated compounds according to the present disclosure successfully eradicated persister cells, even those from antibiotic-resistant strains, within a period of 2 to 24 h. This bactericidal performance exceeded that of conventional antibiotics, which could only achieve a maximum reduction in bacterial population size of approximately 2 log₁₀, even after 24 h of treatment (FIG. 7).

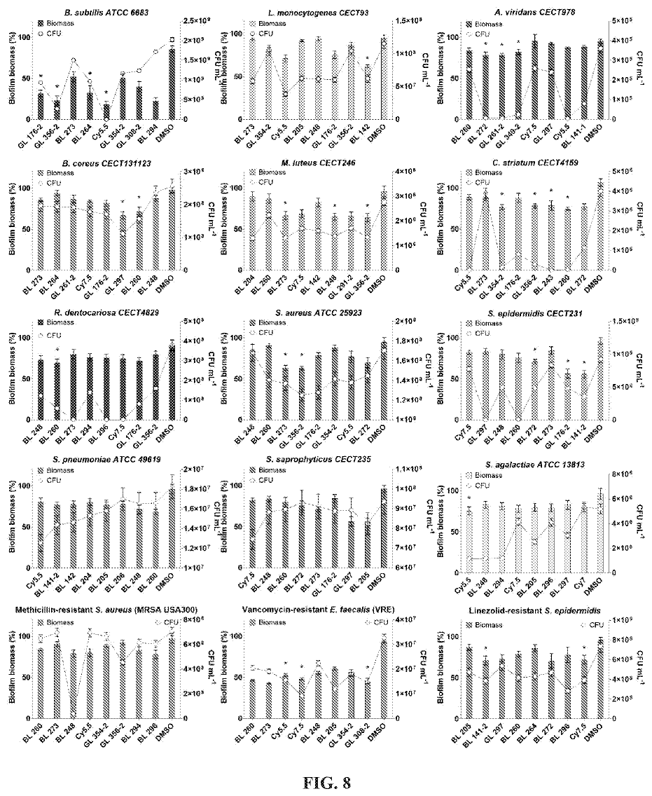

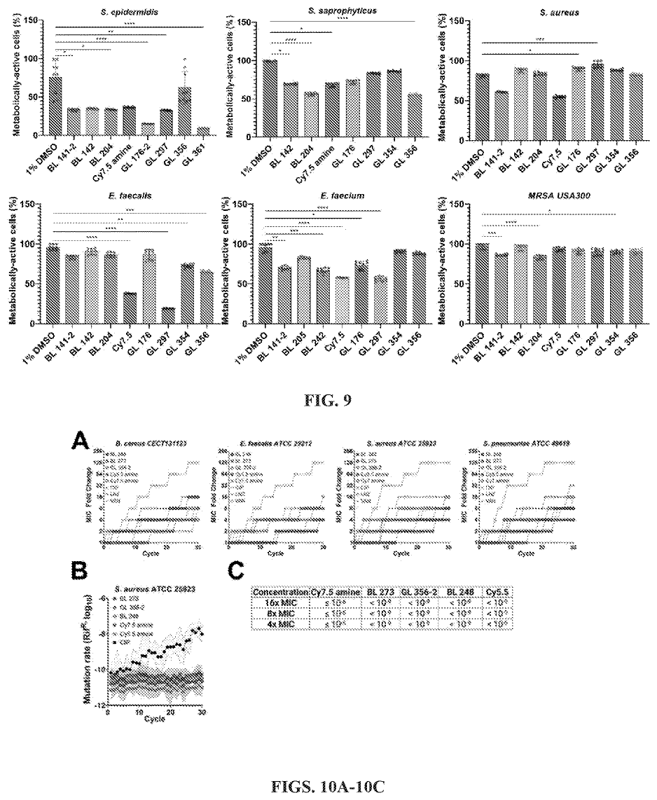

The efficacy of the present charged pi-conjugated compounds in eliminating established biofilms was also investigated by quantifying biofilm biomass using the crystal violet assay and counting colony-forming units (CFU) within treated biofilms. Biofilms are dense microbial communities that adhere to each other and to various surfaces and are encased in a self-generated extracellular polymer matrix. This intricate structure, combined with its unique physiology, makes biofilms exceptionally resistant to antibiotics and immune system responses (Stewart & Costerton, 2001). The ability of charged pi-conjugated compounds to eliminate biofilms varied among bacterial strains. The average reduction in biofilm biomass induced by charged pi-conjugated compounds ranged from 5.8% in L. monocytogenes CECT93 to 52.1% in B. subtilis. Similarly, the charged pi-conjugated compound-induced reduction in viable cells within biofilms ranged from an average of 6.9% in S. saprophyticus CECT235 to 81.5% in R. dentocariosa CECT4829, indicating differential susceptibility of biofilms to charged pi-conjugated compounds (FIG. 8). Similar results were obtained when assessing the impact of charged pi-conjugated compounds on the percentage of metabolically active cells within established biofilms (FIG. 9).

### b. Resistance Development and Mutation Frequency

Both stepwise and single-step methods were used to assess the potential for resistance development upon exposure to charged pi-conjugated compounds. In the stepwise assessment of resistance development, bacterial cells were exposed to increasing concentrations of charged pi-conjugated compounds or conventional antibiotics. The surviving cells were then repeatedly exposed to charged pi-conjugated compounds or antibiotics, and the evolution of MIC over time was monitored. In all the strains studied, cells treated with charged pi-conjugated compounds showed an increase in MIC over 30 treatment cycles ranging from 4 to 16. This was comparable to the results obtained with the antibiotics vancomycin and linezolid but much lower than the results obtained with ciprofloxacin (FIG. 10A). In addition, the mutation frequency (assessed as the frequency of rifampin-resistant colonies) of S. aureus ATCC 25923 subjected to repeated treatment with charged pi-conjugated compound or the conventional antibiotic ciprofloxacin was determined. While the mutation rate for ciprofloxacin showed a marked increase across treatment cycles, the mutation rate in cells repeatedly treated with charged pi-conjugated compound showed a relatively stable and low mutation rate across exposure cycles (FIG. 10B).

For the single-step isolation of resistant mutants, overnight cultures of different bacterial strains (~10¹⁰ CFU mL⁻¹) were plated on LB agar containing 4×, 8×, and 16×MIC of charged pi-conjugated compounds Cy5.5 amine, BL 248, BL 273, Cy7.5 amine, and GL 356-2. The spontaneous mutation rate ranged from ≤10⁻⁶ for plates containing Cy7.5 amine to <10⁻⁹ for the remaining molecules (FIG. 10C). Spontaneous mutants resistant to Cy7.5 amine could be isolated from overnight cultures. However, they could not be propagated on fresh plates supplemented with charged pi-conjugated compounds, and when they could, evaluation of their MIC revealed an MIC indistinguishable from that of the WT (wild-type) strain. This could be attributed to several reasons. It is possible, without being bound by theory, that the mutations acquired in the Cy7.5 amine-resistant mutants were unstable and reverted to the wild-type genotype when transferred to a new environment, resulting in an observed MIC similar to that of the WT strain (Dan et al., 2021). It is also possible, without being bound by theory, that spontaneous mutants may have evolved compensatory mutations or mechanisms that offset the effects of the original mutation. These compensatory mechanisms could, without being bound by theory, restore the fitness and sensitivity to charged pi-conjugated compounds of the bacteria to levels similar to those of the wild type (Martinez & Baquero, 2000). In addition, changes in susceptibility to antibacterials might not, without being bound by theory, always be due to genetic mutations but may result from epigenetic modifications. Such changes may be, without being bound by theory, transient and may not persist in subsequent generations, leading to the observed lack of phenotypic differences in susceptibility to charged pi-conjugated compounds from the WT (Adam et al., 2008).

### c. Influence of Different Physicochemical Factors on the Antibacterial Activity of Charged Pi-Conjugated Compounds

The effects of various physicochemical factors on the antibacterial activity of charged pi-conjugated compounds were studied by measuring the minimum inhibitory concentration (MIC) under varying conditions of temperature, pH, serum, NaCl, and calcium. In addition, the influence of the starting inoculum size was investigated.

The effect of temperature on charged pi-conjugated compounds activity was evaluated by comparing the time-kill curves of two representative bacterial strains at two different temperatures: 25° C. and 37° C. (FIG. 11A). For most charged pi-conjugated compounds, a more significant decrease in the bacterial population size (log N/N₀) was observed at 37° C. than at 25° C. This observation suggests, without being bound by theory, an increased antibacterial efficacy of charged pi-conjugated compounds at a physiologically relevant temperature of 37° C. This increased antibacterial activity of charged pi-conjugated compounds at elevated temperatures may be related, without being bound by theory, to enhanced bacterial metabolism, which may lead to increased charged pi-conjugated compounds uptake or heightened sensitivity to charged pi-conjugated compounds. Additionally, without being bound by theory, temperature fluctuations may affect bacterial membrane fluidity and permeability, potentially affecting the absorption and efficacy of charged pi-conjugated compounds (Hajdu et al., 2010).

The influence of pH on the antibacterial activity of charged pi-conjugated compounds was assessed by determining the MIC of different charged pi-conjugated compounds in CA-MHB adjusted to different pH values. In both S. aureus ATCC 25923 and E. faecalis ATCC 29212, as the pH increased from 5 to 9, there was a general trend of decreasing MIC values for all the molecules tested (FIG. 11B). This indicates, without being bound by theory, that charged pi-conjugated compounds become more effective at inhibiting bacterial growth as the environment becomes more alkaline, potentially, again without being bound by theory, due to improved charged pi-conjugated compounds stability, enhanced uptake, or other pH-sensitive bacterial responses (Thomas et al., 2012; Kinases et al., 2021). The presence of terminal amine groups in charged pi-conjugated compounds may be, without being bound by theory, a reason for the observed pH-dependent activity. Amine groups can accept protons (H⁺) and become positively charged in acidic environments, forming ammonium ions (—NH₃⁺). As the pH increases (becoming more alkaline), amines are less likely to be protonated and tend to exist more in the uncharged state. This change in charge state has several implications. The charge of a molecule can influence its ability to cross bacterial cell membranes. Uncharged molecules generally tend to pass through lipid bilayers more easily than charged molecules. Thus, under alkaline conditions, where more charged pi-conjugated compounds exist in the uncharged state, they might have enhanced penetration into bacterial cells. The binding of a molecule to its target site in bacteria can also be affected by its charge. The protonation state of charged pi-conjugated compounds may, without being bound by theory, determine its binding affinity for specific bacterial targets. Protonation or deprotonation of amine groups can lead to, without being bound by theory, conformational changes in the charged pi-conjugated compounds molecule, which can influence its interaction with bacterial targets. The chemical stability of charged pi-conjugated compounds can also be pH dependent. Under certain pH conditions, these molecules, without being bound by theory, may be more stable and less prone to degradation, leading to increased effectiveness.

The influence of serum on the antibacterial activity of charged pi-conjugated compounds was investigated by testing the effect of increasing serum concentrations on charged pi-conjugated compound MIC (FIG. 11C). As the serum concentration increased from 0 to 50%, there was a noticeable increase in the MIC. This suggests, without being bound by theory, that charged pi-conjugated compounds become less effective when higher serum concentrations are present. One possible reason for this, again without being bound by theory, could be reduced bioavailability: serum proteins might bind to the charged pi-conjugated compounds, thereby decreasing their free concentration and, subsequently, their ability to act against pathogens. Another potential explanation, still without being bound by theory, is the inactivation of charged pi-conjugated compounds. Some components found in the serum may either chemically inactivate charged pi-conjugated compounds or promote their degradation. Finally, still without being bound by theory, serum might provide a protective environment for bacteria, making them less susceptible to the antibacterial activity of charged pi-conjugated compounds (Kaplan et al., 2013).

Next, the effect of NaCl concentration on the activity of charged pi-conjugated compounds was tested by determining the MIC of CA-MHB supplemented with increasing concentrations of NaCl (FIG. 11D). In the case of S. aureus, the MIC either increased or remained stable, whereas in E. faecalis, the MIC was either stable or decreased with increasing NaCl concentration. Variations in MIC with increasing concentrations of NaCl could be attributed, without being bound by theory, to the influence of NaCl on the ionic environment, which might affect bacterial membrane permeability or charged pi-conjugated compound stability, subsequently altering its antibacterial efficacy. The different responses of the two bacterial strains to NaCl suggest, without being bound by theory, inherent differences in their responses to osmotic stress or variations in their cell wall structures. The presence of free amine groups on charged pi-conjugated compounds can also play a pivotal role in influencing the MIC in the presence of NaCl. Without being bound by any particular theory, the ionic strength of the environment, altered by the addition of NaCl, may affect the electrostatic interactions between the positively charged amine groups on the molecule and the negatively charged bacterial cell surface. As a result, the binding affinity of charged pi-conjugated compound to bacterial cells might be impacted by either enhancing or reducing the ability of the molecule to exert its antibacterial activity. The electrostatic shield formed by the salt may, again without being bound by theory, hinder the interaction of the charged pi-conjugated compound molecule with its target on the bacterial cell, thereby requiring higher concentrations of the molecule to achieve the same inhibitory effect, leading to an elevated MIC. Conversely, still without being bound by theory, certain scenarios might also see a more facilitated interaction owing to altered charge distributions (Li et al., 2021; Yoon et al., 2013).

Subsequently, the effect of increasing the calcium ion concentration was investigated (FIG. 11E). The different responses of the two strains to increased calcium concentrations illustrate the multiple effects of calcium on MIC, potentially mediated by its impact on bacterial cell wall stability, uptake mechanisms, or efflux pump activity (Xie & Yang, 2016).

Finally, the influence of inoculum size on the antibacterial activity of charged pi-conjugated compounds was assessed (FIG. 11F). Increasing the inoculum size, that is, the initial number of bacterial cells exposed to charged pi-conjugated compounds, was found to increase the concentration of compound needed to inhibit growth. This phenomenon can be explained, without being bound by theory, by considering the antibacterial action of charged pi-conjugated compounds at the population level. When a larger starting inoculum of bacteria is present, even if charged pi-conjugated compounds can kill some cells, the remaining viable cells can proliferate and restore the population. Therefore, a higher concentration of charged pi-conjugated compound is needed to inhibit growth. At high cell densities, individual cells may also, without being bound by theory, be exposed to lower per-cell compound concentrations in their microenvironments (Morrissey & George, 1999).

### d. Mechanism of Action Studies

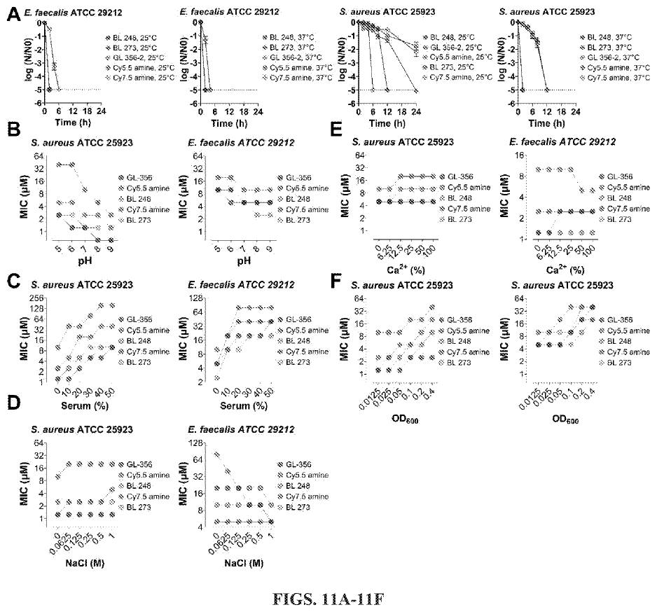

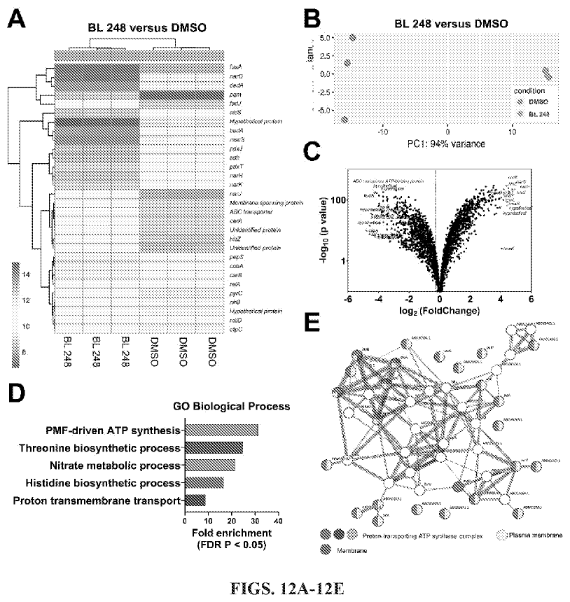

The mechanism of action of charged pi-conjugated compounds was investigated using RNA sequencing (RNA-seq) by treating S. aureus with a sublethal concentration of charged pi-conjugated compound BL 248 (FIG. 12). RNA-seq analysis of S. aureus treated with sublethal concentrations of charged pi-conjugated compound revealed distinct transcriptional responses. Several genes showed pronounced up- or downregulation in response to treatment with charged pi-conjugated compounds (FIG. 12A). Principal component analysis (PCA) further differentiated the gene expression profiles between treatments. Remarkably, 94% of the variance was captured by PC1, indicating significant differences in the gene expression profiles between the treated and untreated samples (FIG. 12B). The protein-protein interaction network indicated proteins related to the proton-transporting ATP synthase complex and membranes as critical regulatory and metabolic nodes affected by treatment with charged pi-conjugated compounds (FIG. 12C). Gene Ontology (GO) Biological Process analysis revealed that the processes most affected by treatment with charged pi-conjugated compounds were proton motive force-driven ATP synthesis, threonine biosynthesis, nitrate metabolism, histidine biosynthesis, and proton transmembrane transport (FIG. 12D, FIG. 12E). These results suggest, without being bound by theory, that treatment with charged pi-conjugated compounds affects the energy generation pathways, amino acid synthesis, and ion transport mechanisms.

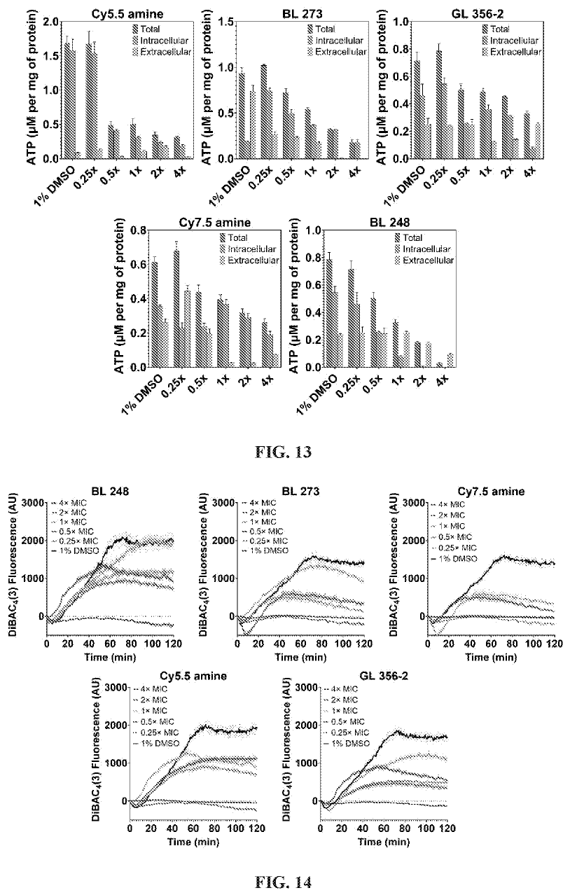

To investigate the effect of treatment with charged pi-conjugated compounds on bacterial energy metabolism, the effect of increasing concentrations of charged pi-conjugated compounds on the total ATP levels (both intracellular and extracellular) in S. aureus was examined (FIG. 13). A consistent decrease in total ATP levels was observed with treatment with charged pi-conjugated compound, with both intracellular and extracellular ATP levels decreasing with increasing charged pi-conjugated compound concentration. This suggests, without being bound by theory, that charged pi-conjugated compounds may inhibit bacterial metabolic pathways associated with ATP production, such as oxidative phosphorylation and glycolysis, thereby affecting bacterial growth and viability.

The effect of different charged pi-conjugated compounds on the membrane potential was then investigated using the fluorescent probe DiBAC4 (5). DiBAC4 (5) is a voltage-sensitive fluorescent dye commonly used to measure changes in membrane potential (Clementi et al., 2014). Most of the charged pi-conjugated compounds tested had detectable effects on the membrane potential of S. aureus. Higher charged pi-conjugated compound concentrations consistently resulted in greater membrane depolarization, indicating a dose-dependent effect (FIG. 14). This suggests that charged pi-conjugated compounds might affect the integrity of the bacterial membrane or disrupt ion channels or transporters, preventing bacteria from maintaining their electrochemical proton gradient. These observations are consistent with the RNA-seq data.

### e. Interactions with Conventional Antibiotics

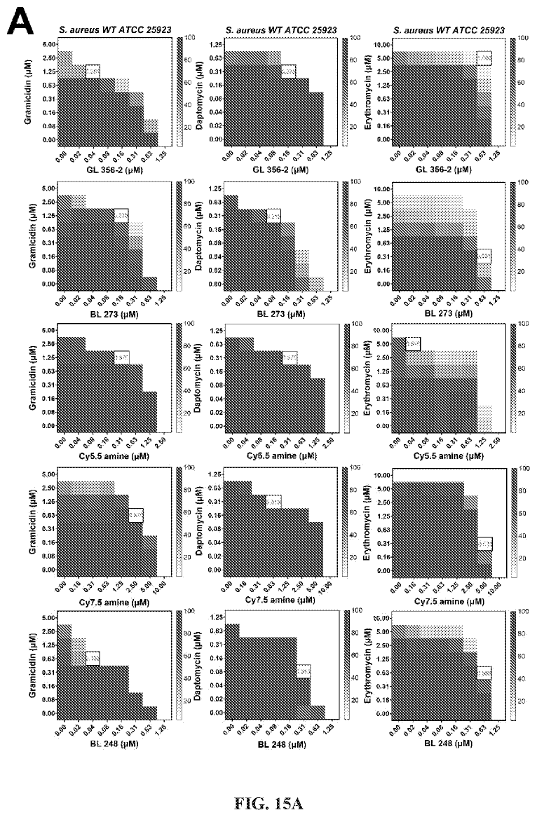

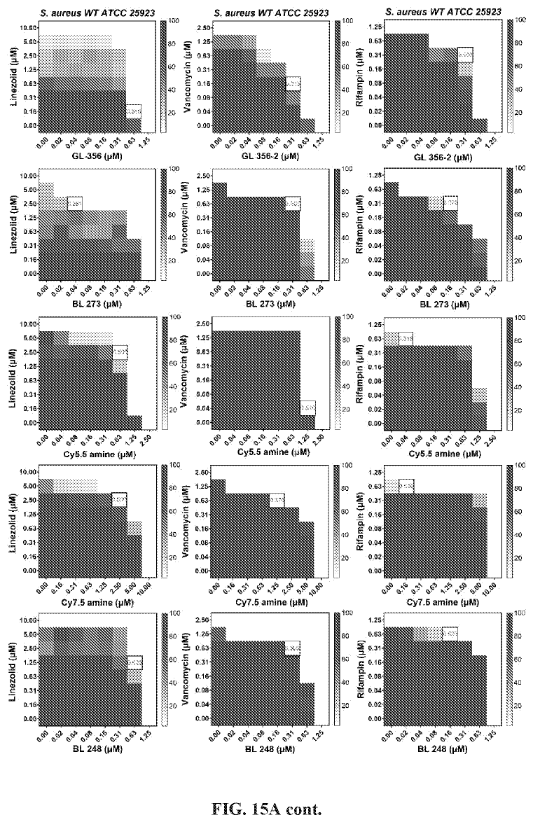

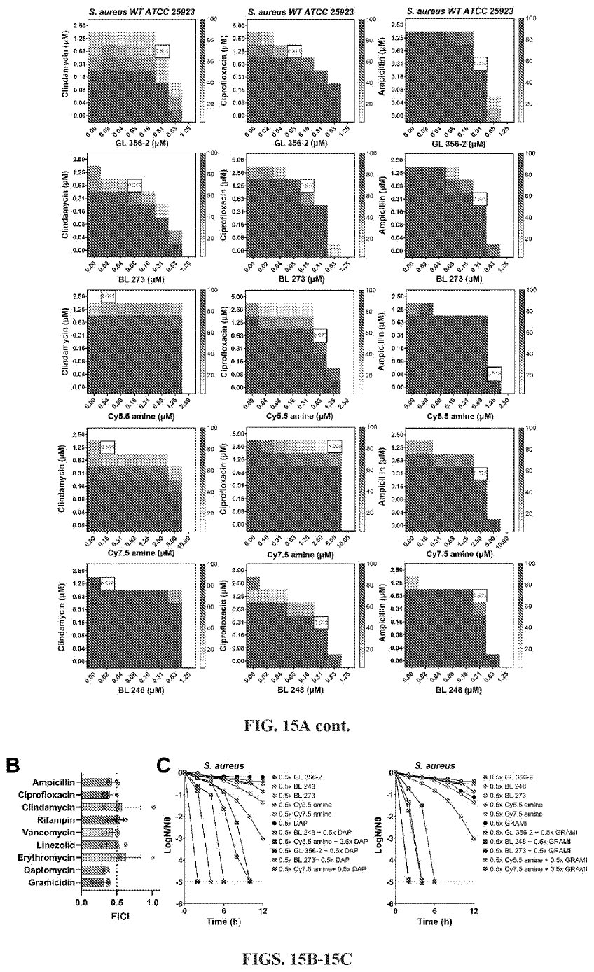

The interactions of charged pi-conjugated compounds with various conventional antibiotics with different mechanisms of action were evaluated using a checkerboard assay. In general, combinations of charged pi-conjugated compounds with conventional antibiotics yielded fractional inhibitory concentration index (FICI) values of ≤0.5, indicating a synergistic interaction between conventional antibiotics and various charged pi-conjugated compounds (FIG. 15A). Such consistent synergistic interactions indicate that the mechanism of action of charged pi-conjugated compounds differs from that of conventional antibiotics, thus allowing them to collaboratively inhibit bacterial growth. Of note, the interaction between charged pi-conjugated compounds and the antibiotics gramicidin and daptomycin had the lowest average FICI, indicating a particularly strong synergistic relationship (FIG. 15B). The interaction between charged pi-conjugated compounds and antibiotics was further investigated in time-kill assays. When S. aureus was treated with sublethal concentrations (0.5×MIC) of individual charged pi-conjugated compounds, gramicidin, or daptomycin, a maximum reduction in bacterial population size of ≈3 log₁₀ was observed even after 12 h of treatment. In contrast, combinations of sublethal concentrations of charged pi-conjugated compounds with antibiotics resulted in complete eradication of the bacterial population (i.e., ~5 log₁₀ reduction) within a range of 2 to 10 h (FIG. 15C).

Gramicidin and daptomycin primarily target bacterial membranes. Daptomycin is known for its ability to integrate into bacterial membranes in a calcium-dependent manner and trigger potassium ion efflux from bacterial cells. This phenomenon disrupts the membrane potential and can lead to cell death (Silverman et al., 2003). Conversely, gramicidin forms ion channels in bacterial membranes, facilitating ion movement and thus disrupting the proton and ion gradients that are essential for bacterial functionality (Wenzel et al., 2018). The observation of strong synergistic interactions between charged pi-conjugated compounds and both gramicidin and daptomycin suggests, without being bound by theory, that these antibiotics may have mechanisms of action that complement those of charged pi-conjugated compounds. In addition, one antibacterial agent could enhance the cellular uptake or retention of another, such that their combined effect is more potent than that of the individual agents. Thus, the synergistic effect of charged pi-conjugated compounds with daptomycin and gramicidin could result, without being bound by theory, from a multifaceted attack on the bacterial membrane, culminating in profound dysfunction and leading to bacterial death.

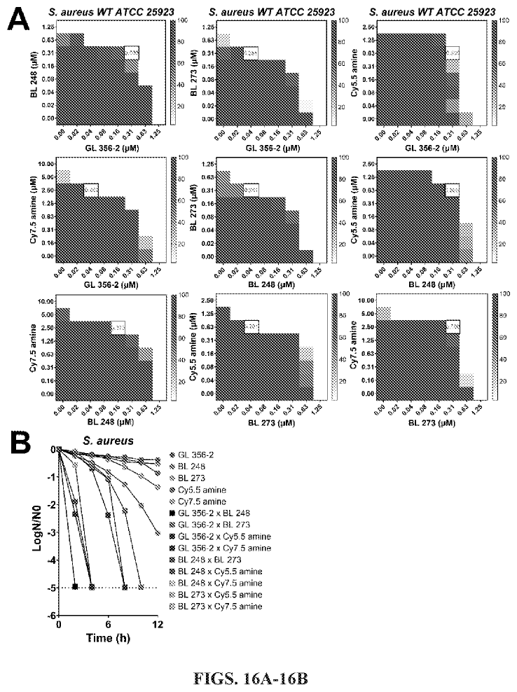

The checkerboard assay was also used to investigate the interactions between different charged pi-conjugated compounds. Interestingly, some molecules exhibited synergistic interactions (FICI≤0.5) (FIG. 16A). Accordingly, time-kill curves for S. aureus treated with combinations of sublethal charged pi-conjugated compound concentrations resulted in the complete eradication of the bacteria (≈5 log₁₀ reduction) in only 2 h, in contrast to a maximum reduction of ≈3 log₁₀ even after 12 h of treatment with equivalent concentrations of each individual compound (FIG. 16B). These results suggest, without being bound by theory, that different charged pi-conjugated compound derivatives may possess complementary mechanisms of action that enhance their combined antimicrobial activities. It is conceivable, again without being bound by theory, that these molecules may bind to different components of the membrane or induce unique membrane conformational changes that collectively exert a more pronounced disruptive effect. These variants could also have, still without being bound by theory, different binding affinities or kinetics. As a non-limiting illustrative example, one variant might bind rapidly and destabilize the membrane, whereas another variant might cause slower but more sustained integration. Their combined effects could, without being bound by theory, in some embodiments increase the antimicrobial efficacy. In addition, in some embodiments, combined variants could affect membrane function by altering membrane protein composition or inhibiting membrane-based efflux pumps, thus amplifying the perturbation through their individual interactions.

### f. Biocompatibility

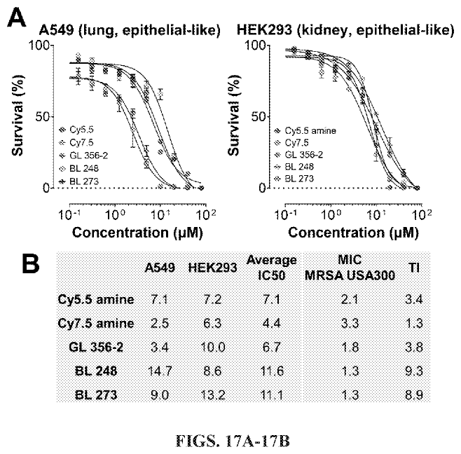

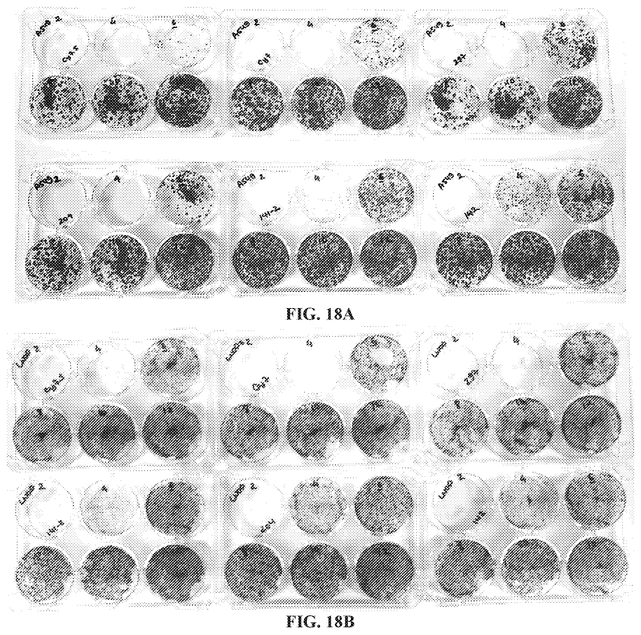

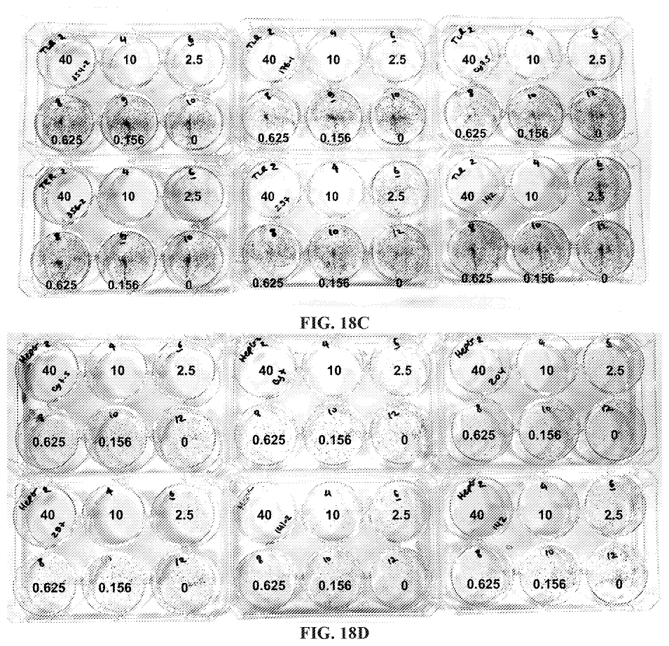

The biocompatibility of charged pi-conjugated compounds with mammalian cell lines was evaluated by studying the effects of different charged pi-conjugated compound concentrations on the viability of two mammalian cell lines: A549 (epithelial-like lung cells) and HEK293 (epithelial-like kidney cells). An inverse relationship between charged pi-conjugated compound concentration and viability was observed in both cell lines: as the charged pi-conjugated compound concentration increased, the survival rate decreased. This illustrates some cytotoxic effects of these molecules, particularly at high concentrations (FIG. 17A). These results were consistent with those obtained using complementary methodologies and in additional mammalian cell lines (FIG. 18, FIG. 19). Dose-response curves were used to determine IC₅₀ values, that is, the concentration of charged pi-conjugated compound needed to inhibit cell viability by 50%. For the A549 cell line, IC₅₀ values ranged from 2.5 μM (GL 356-2) to 14.7 μM (BL 248), while for the HEK293 cell line, IC₅₀ values ranged from 6.3 μM (Cy7.5 amine) to 13.7 μM (BL 273). The safety of the molecules was further quantified by calculating the therapeutic index, i.e., the ratio between the concentration at which a drug becomes toxic (IC₅₀) and the concentration at which it is effective (MIC). The MIC of MRSA USA300 was used to calculate the therapeutic index. MRSA USA300 is a major contributor to community-associated MRSA infections, including skin and soft tissue infections, bacteraemia, endocarditis, bone and joint infections and nosocomial infections. MRSA USA300 is notable for its increased multidrug resistance, including resistance to the antibiotics erythromycin, levofloxacin, mupirocin, and tetracycline. Additionally, its high transmissibility has been associated with outbreaks in various settings, including prisons and athletic teams. The therapeutic index ranged from 1.3 for Cy7.5 amine to 9.3 for BL 248 (FIG. 17B).

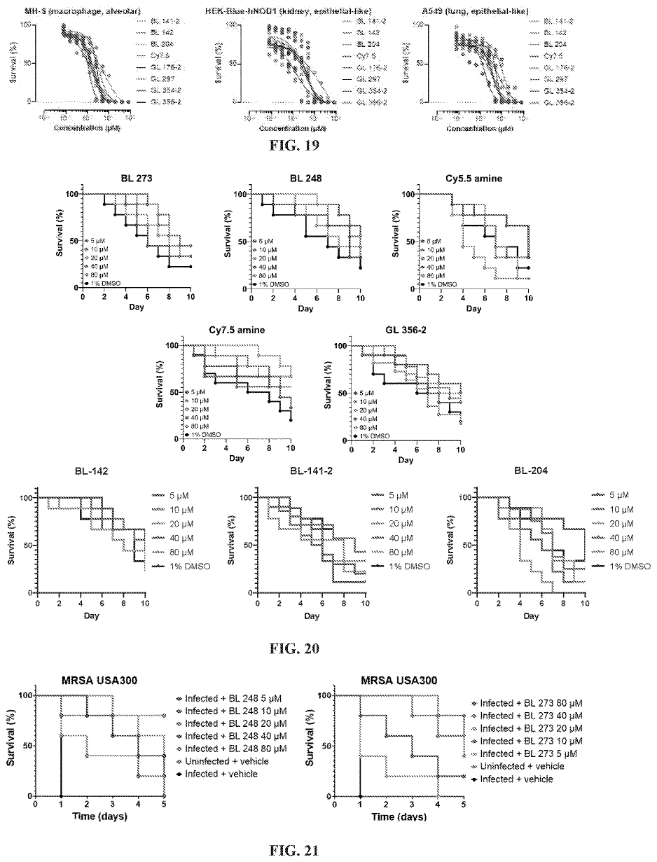

The in vivo biocompatibility of charged pi-conjugated compounds was further investigated using the invertebrate model Galleria mellonella (Serrano et al., 2023). Worms were injected with increasing concentrations of aqueous solutions of charged pi-conjugated compounds or vehicle, and their survival was monitored. The results revealed an interesting observation: intermediate charged pi-conjugated compounds concentrations appeared to improve survival rates compared to the vehicle control (1% DMSO) (FIG. 20). These results suggest, without being bound by theory, the possible induction of a hormetic response, in which moderate doses of charged pi-conjugated compounds could provide a protective advantage, possibly by activating the worm's innate defense mechanisms.

### g. In Vivo Anti-Infective Activity

The in vivo anti-infective capacity of charged pi-conjugated compounds was evaluated in the invertebrate model G. mellonella infected with MRSA USA300. The survival curves shown in FIG. 21 illustrate the efficacy of two charged pi-conjugated compounds, BL 248 and BL 273, against MRSA USA300 infection in G. mellonella. With increasing concentrations of both molecules, there was a discernible improvement in survival compared with the vehicle-treated control group. Specifically, in the case of BL 248, doses between 5 and 40 μM extended the survival of infected worms from as little as one day in vehicle-treated worms to four or more days in charged pi-conjugated compound-treated worms. In the case of BL 273, charged pi-conjugated compound treatment extended survival from one day in vehicle-treated infected worms to three or more days in worms treated with 10-80 μM charged pi-conjugated compound. These results highlight the promising anti-infective potential of both charged pi-conjugated compounds against MRSA strain USA300.

### h. Materials and Methods

i Synthetic chemistry

The synthesis and characterization of the charged pi-conjugated compounds used in this study have been described elsewhere, including above (Ayala-Orozco et al., 2023). FIG. 1 shows the chemical structures of these molecules.

### ii Strains and Reagents

The complete list of the strains used in this study and their provenances are shown in Table 2. Unless otherwise stated, all reagents were purchased from MedChem Express (Princeton, NJ, USA), Cayman Chemical Company (Ann Arbor, MI, USA), or MilliporeSigma (St. Louis, MO, USA) and dissolved in 100% DMSO or an appropriate solvent according to the manufacturer's instructions.

TABLE 2
List of strains used in this study, their provenance, designation, and growth conditions.

| | | Strain | | Temp. |
|---|---|---|---|---|
| Species | Source | designation | Media base | (° C.) |
| Acinetobacter baumannii | ATCC | ATCC 17978 | CA-MH | 37 |
| Aerococcus urinae | Clinical isolate | 10144921 | BHI | 37 |
| Aerococcus urinae | Clinical isolate | 11161662 | BHI | 37 |
| Aerococcus urinae | Clinical isolate | 1179513 | BHI | 37 |
| Aerococcus urinae | Clinical isolate | 12089666 | BHI | 37 |
| Aerococcus viridans | CECT | CECT978 | BHI | 37 |
| Bacillus cereus | Clinical isolate | 12295949 | CA-MH | 30 |
| Bacillus cereus | Clinical isolate | 11426444 | CA-MH | 30 |
| Bacillus cereus | Clinical isolate | 19244888 | CA-MH | 30 |
| Bacillus cereus | Clinical isolate | 19267672 | CA-MH | 30 |
| Bacillus cereus | CECT | CECT131123 | CA-MH | 30 |
| Bacillus subtilis | Clinical isolate | ATCC 6683 | CA-MH | 30 |
| Corynebacterium amycolatum | Clinical isolate | 11349130 | BHI | 37 |
| Corynebacterium amycolatum | Clinical isolate | 18444472 | BHI | 37 |
| Corynebacterium jeikeium | Clinical isolate | 11275057 | BHI | 37 |
| Corynebacterium jeikeium | Clinical isolate | 10287592 | BHI | 37 |
| Corynebacterium jeikeium | Clinical isolate | 11546927 | BHI | 37 |
| Corynebacterium jeikeium | Clinical isolate | 16149642 | BHI | 37 |
| Corynebacterium striatum | Clinical isolate | 10018762 | BHI | 37 |
| Corynebacterium striatum | Clinical isolate | 19194638 | BHI | 37 |
| Corynebacterium striatum | CECT | CECT4159 | BHI | 37 |
| Enterococcus avium | Clinical isolate | 12314658 | CA-MH | 37 |
| Enterococcus avium | Clinical isolate | 11151402 | CA-MH | 37 |
| Enterococcus avium | Clinical isolate | 12373133 | CA-MH | 37 |
| Enterococcus casseliflavus | Clinical isolate | 11309015 | CA-MH | 37 |
| Enterococcus casseliflavus | Clinical isolate | 12090134 | CA-MH | 37 |
| Enterococcus casseliflavus | Clinical isolate | 12387844 | CA-MH | 37 |
| Enterococcus faecalis | ATCC | ATCC 29212 | CA-MH | 37 |
| Enterococcus faecalis | Clinical isolate | 12404463 | CA-MH | 37 |
| Enterococcus faecalis | Clinical isolate | 12439547 | CA-MH | 37 |
| Enterococcus faecium | Clinical isolate | | CA-MH | 37 |
| Enterococcus faecium | Clinical isolate | 12283124 | CA-MH | 37 |
| Enterococcus faecium | Clinical isolate | 12430234 | CA-MH | 37 |
| Enterococcus faecium | CECT | CECT410 | CA-MH | 37 |
| Enterococcus faecium (VRE) | Clinical isolate | 11375599 | CA-MH | 37 |
| Enterococcus faecium (VRE) | Clinical isolate | 12341224 | CA-MH | 37 |
| Enterococcus gallinarum | Clinical isolate | 12049896 | CA-MH | 37 |
| Enterococcus gallinarum | Clinical isolate | 12373132 | CA-MH | 37 |
| Enterococcus gallinarum | Clinical isolate | 16787814 | CA-MH | 37 |
| Escherichia coli | ATCC | MG1655 | CA-MH | 37 |
| Listeria monocytogenes | Clinical isolate | 11318353 | CA-MH | 37 |
| Listeria monocytogenes | Clinical isolate | 17141367 | CA-MH | 37 |
| Listeria monocytogenes | Clinical isolate | 11550796 | CA-MH | 37 |
| Listeria monocytogenes | Clinical isolate | 11569079 | CA-MH | 37 |
| Listeria monocytogenes | Clinical isolate | 184449443 | CA-MH | 37 |
| Listeria monocytogenes | CECT | CECT93 | CA-MH | 37 |
| Micrococcus luteus | CECT | CECT246 | CA-MH | 30 |
| MRSA | Clinical isolate | CC22 ST22 | CA-MH | 37 |
| | | 93658844 | | |
| MRSA | Clinical isolate | CC30 ST30 | CA-MH | 37 |
| | | 18047170 | | |
| MRSA | Clinical isolate | C30 ST714417349 | CA-MH | 37 |
| MRSA | Clinical isolate | CC5 | CA-MH | 37 |
| | | ST12511154254 | | |
| MRSA | Clinical isolate | CC8 ST8 18021220 | CA-MH | 37 |
| MRSA | Clinical isolate | USA300 | CA-MH | 37 |
| MRSA | Clinical isolate | 11341631 | CA-MH | 37 |
| MRSA | Clinical isolate | 11356635 | CA-MH | 37 |
| MRSA | Clinical isolate | 16251949 | CA-MH | 37 |
| MRSA | Clinical isolate | 18414447 | CA-MH | 37 |
| MRSA | Clinical isolate | 10518755 | CA-MH | 37 |
| MRSA | Clinical isolate | 18170396 | CA-MH | 37 |
| MRSA | Clinical isolate | 16111230 | CA-MH | 37 |
| MRSA | Clinical isolate | 11015131 | CA-MH | 37 |
| MRSA | Clinical isolate | 11154628 | CA-MH | 37 |
| MRSA | Clinical isolate | 11360439 | CA-MH | 37 |
| MRSA | Clinical isolate | 11346606 | CA-MH | 37 |
| MRSA | Clinical isolate | 11162355 | CA-MH | 37 |
| MRSA | Clinical isolate | 11173419 | CA-MH | 37 |
| Mycobacterium smegmatis | CECT | CECT3017 | Middlebrook | 37 |
| | | | 7H9 | |
| Propionibacterium acnes | Clinical isolate | 11286299 | BHI | 37 |
| Propionibacterium acnes | Clinical isolate | 19392786 | BHI | 37 |
| Propionibacterium acnes | Clinical isolate | 11019410 | BHI | 37 |
| Propionibacterium acnes | Clinical isolate | 10506635 | BHI | 37 |
| Pseudomonas aeruginosa | ATCC | ATCC 27853 | CA-MH | 37 |
| Rothia dentocariosa | Clinical isolate | 12149062 | BHI | 37 |
| Rothia dentocariosa | CECT | CECT4829 | BHI | 37 |
| Rothia mucilaginosa | Clinical isolate | 11247489 | BHI | 37 |
| Rothia mucilaginosa | Clinical isolate | 41052881 | BHI | 37 |
| Staphylococcus aureus | ATCC | ATCC 25923 | CA-MH | 37 |
| Staphylococcus aureus | ATCC | ATCC 43300 | CA-MH | 37 |
| Staphylococcus epidermidis | Clinical isolate | 11326848 | CA-MH | 37 |
| Staphylococcus epidermidis | Clinical isolate | 12055721 | CA-MH | 37 |
| Staphylococcus epidermidis | CECT | CECT231 | CA-MH | 37 |
| Staphylococcus epidermidis | Clinical isolate | 12158296 | CA-MH | 37 |
| LnzR | | | | |
| Staphylococcus epidermidis | Clinical isolate | 12180066 | CA-MH | 37 |
| LnzR | | | | |
| Staphylococcus epidermidis | Clinical isolate | 12095352 | CA-MH | 37 |
| LnzR | | | | |
| Staphylococcus epidermidis | Clinical isolate | 12073113 | CA-MH | 37 |
| LnzR | | | | |
| Staphylococcus epidermidis | Clinical isolate | 12108067 | CA-MH | 37 |
| LnzR | | | | |
| Staphylococcus epidermidis | Clinical isolate | 12114501 | CA-MH | 37 |
| LnzR | | | | |
| Staphylococcus epidermidis | Clinical isolate | 12150001 | CA-MH | 37 |
| LnzR | | | | |
| Staphylococcus epidermidis | Clinical isolate | 12115604 | CA-MH | 37 |
| LnzR | | | | |
| Staphylococcus epidermidis | Clinical isolate | 12159482 | CA-MH | 37 |
| LnzR | | | | |
| Staphylococcus saprophyticus | Clinical isolate | 12375721 | CA-MH | 37 |
| Staphylococcus saprophyticus | Clinical isolate | 10344853 | CA-MH | 37 |
| Staphylococcus saprophyticus | Clinical isolate | 10401983 | CA-MH | 37 |
| Staphylococcus saprophyticus | Clinical isolate | 10277449 | CA-MH | 37 |
| Staphylococcus saprophyticus | Clinical isolate | 12290287 | CA-MH | 37 |
| Staphylococcus saprophyticus | CECT | CECT235 | CA-MH | 37 |
| Streptococcus agalactiae | ATCC | ATCC 13813 | BHI | 37 |
| Streptococcus agalactiae | Clinical isolate | 95147585 | BHI | 37 |
| Streptococcus agalactiae | Clinical isolate | 11510705 | BHI | 37 |
| Streptococcus agalactiae | Clinical isolate | 12100628 | BHI | 37 |
| Streptococcus agalactiae | Clinical isolate | 12184597 | BHI | 37 |
| Streptococcus agalactiae | Clinical isolate | 12326715 | BHI | 37 |
| Streptococcus agalactiae | Clinical isolate | 12421803 | BHI | 37 |
| Streptococcus anginosus | Clinical isolate | | BHI | 37 |
| Streptococcus canis | Clinical isolate | | BHI | 37 |
| Streptococcus constellatus | Clinical isolate | | BHI | 37 |
| Streptococcus dysgalactiae | Clinical isolate | | BHI | 37 |
| Streptococcus mitis | Clinical isolate | | BHI | 37 |
| Streptococcus pneumoniae | ATCC | ATCC 49619 | BHI | 37 |
| Streptococcus pneumoniae | Clinical isolate | 50058224 | BHI | 37 |
| Streptococcus pneumoniae | Clinical isolate | 10047196 | BHI | 37 |
| Streptococcus pneumoniae | Clinical isolate | 10351848 | BHI | 37 |
| Streptococcus pneumoniae | Clinical isolate | 10076393 | BHI | 37 |
| Streptococcus pneumoniae | Clinical isolate | 10118034 | BHI | 37 |
| Streptococcus pneumoniae | Clinical isolate | 10115656 | BHI | 37 |
| Streptococcus pneumoniae | Clinical isolate | 10084553 | BHI | 37 |
| Streptococcus pneumoniae | Clinical isolate | 10122093 | BHI | 37 |
| Streptococcus pneumoniae | Clinical isolate | 19418269 | BHI | 37 |
| Streptococcus pneumoniae | Clinical isolate | 29456285 | BHI | 37 |
| Streptococcus pneumoniae | Clinical isolate | 10152068 | BHI | 37 |
| Streptococcus pyogenes | | CCSEIMC | BHI | 37 |
| Streptococcus pyogenes | Clinical isolate | 95626100 | BHI | 37 |
| Streptococcus pyogenes | Clinical isolate | 12541324 | BHI | 37 |
| Streptococcus pyogenes | Clinical isolate | 95617030 | BHI | 37 |
| Streptococcus pyogenes | Clinical isolate | 16787388 | BHI | 37 |
| Streptococcus pyogenes | Clinical isolate | 12544990 | BHI | 37 |
| Streptococcus pyogenes | Clinical isolate | 12541307 | BHI | 37 |
| Streptococcus pyogenes | Clinical isolate | 12543313 | BHI | 37 |
| Streptococcus pyogenes | Clinical isolate | 12537563 | BHI | 37 |

CA-MH: Cation-adjusted Mueller-Hinton.
BHI: Brain Heart Infusion

### iii Preparation of Cell Suspensions

Bacterial cells retrieved from glycerol stocks stored at −80° C. were streaked onto blood agar plates to isolate individual colonies. One colony was then cultured overnight with agitation at 220 rpm at the appropriate temperature and in the liquid media specified in Table 2. Then, the overnight culture was diluted 1:50 in fresh medium and grown to an optical density at 600 nm (OD₆₀₀) of approximately 1. The cells were then centrifuged at 5,000×g for 10 min, rinsed, and resuspended in phosphate-buffered saline (1×PBS) to a density of approximately 10⁶ CFU mL⁻¹.

### iv Irradiation Setup

The cell suspensions prepared as described above were transferred to a 6-well plate and positioned at the center of a 730 nm light-emitting diode (UHP—F-730 nm, Prizmatix, Israel). The distance from the light source was adjusted to achieve an intensity of 80 mW cm-2, which was verified using a ThorLabs Sensor S415C (Thorlabs, Newton, MA, USA). The samples were irradiated for 10 min at a fluence of 48 J/cm².

v Minimum inhibitory concentration

The broth microdilution method was used to determine the MIC of charged pi-conjugated compounds according to the CLSI guidelines. Charged pi-conjugated compound stock solutions (16 mM) were prepared in dimethyl sulfoxide (DMSO). Serial twofold dilutions of the charged pi-conjugated compounds were freshly prepared using the appropriate liquid media (Table 2), and 100 μL of each dilution was added to the appropriate wells of 96-well microtiter plates. The bacterial inoculum was further diluted in liquid media, and 100 μL was added to each well, resulting in a final cell density of ≈5×10⁵ CFU mL⁻¹. Negative controls (without cells) and positive controls (without charged pi-conjugated compounds) were also included. After incubation at the appropriate temperature (Table 2) for 16-20 hours, the OD₆₀₀ was measured using a microplate reader (BioTek Instruments Inc., Winooski, VT, USA). The minimal inhibitory concentration (MIC) was defined as the lowest concentration of the compound resulting in complete bacterial growth arrest. The minimal bactericidal concentration (MBC) was defined as the lowest concentration of the molecule that killed 99.9% of the inoculum. A molecule was determined to be bactericidal if the ratio of the MBC to the MIC was less than 4 to 6. All experiments were repeated at least three times.

### vi Growth Curves

Overnight bacterial cultures were prepared in CA-MH broth to achieve a density of approximately 10⁶ CFU mL⁻¹, as previously described. The cells were treated with 1% DMSO or increasing concentrations of charged pi-conjugated compounds. These samples were transferred to a 96-well microtiter plate (Corning, NY, USA) and covered with mineral oil to reduce evaporation during the incubation. Growth trajectories were recorded at 37° C. by monitoring OD₆₀₀ every 10 min using a microplate reader (BioTek Instruments Inc., Winooski, VT, USA). Each experiment was repeated at least three times.

### vii Time-Kill Experiments

Reference bacterial strains representing each species from Table 2, including antibiotic-resistant strains known for their clinical significance, were used for time-kill assays with both exponentially growing cells and persister cells.

Exponential cells from fresh overnight cultures on Blood Agar plates were suspended in the appropriate media (Table 2) to achieve an OD₆₀₀ of approximately 0.1. To isolate persister cells, bacterial cultures were grown with agitation (220 rpm) at the appropriate temperature and in the appropriate media (Table 2) until they reached an OD₆₀₀ of approximately 0.1. The cells were then diluted 1:1000 in fresh media and cultured for an additional 16 h under the same conditions. Linezolid was added at a concentration equivalent to 10×MIC to eliminate antibiotic-susceptible cells. After 24 h of treatment, the cells were harvested, washed to remove residual antibiotics, and diluted in fresh media (Table 2) to approximately 10⁶ CFU mL⁻¹ (Santos et al., 2022).

Charged pi-conjugated compounds were added at concentrations of 4×, 8×, and 16× MIC. Samples treated with conventional antibiotics (linezolid and vancomycin) were used as controls. After antibiotic exposure, aliquots were collected at different time points, diluted in PBS, and plated on the appropriate agar media (Table 2). Colony counts were performed after 18-24 hours of incubation at the appropriate temperature (Table 2). Time-kill curves were generated, and bactericidal activity was defined as a decrease in CFU mL⁻¹ by ≥3 log 10 compared to the initial inoculum. Only dilutions yielding 10-100 colonies were used. The results were expressed as log(N/N0), where N represents the CFU mL⁻¹ at each observation time point, and NO represents the initial CFU mL⁻¹ of each sample.

viii Antibiofilm Activity

The bacterial strains were cultured overnight in the appropriate media at the appropriate temperature (Table 2) and shaken at 200 rpm. The cultures were adjusted to an optical density (OD₆₀₀) of ≈0.1. Subsequently, 100 μL of these cultures was transferred to 96-well polystyrene microtiter plates. After stationary incubation at 30° C. or 37° C. (Table 2) for 24 h to form biofilms, the non-adherent cells were washed with phosphate-buffered saline (PBS). The established biofilms were then exposed to 8× the MIC of charged pi-conjugated compounds or reference antibiotics in the appropriate media (Table 2). After 24 h, the supernatant was removed, and the biofilms were washed with PBS and dried (Santos et al., 2022).

For quantification, biofilms were stained with 0.1% (w/v) crystal violet solution (100 μL) at room temperature for 30 min. The stain was then removed, and the wells were washed with water. The bound dye was dissolved in 30% acetic acid, and the absorbance was measured at 550 nm using a microplate reader (BioTek Instruments Inc., Winooski, VT, USA). In another batch of similarly treated samples, biofilms were mechanically disrupted in PBS, diluted, and plated on the appropriate media to count viable cells. These results, expressed as surviving CFU mL⁻¹, were compared with those of the untreated controls. In yet another batch of similarly treated samples, metabolically active cells were assessed by quantifying ATP levels in biofilms using the BacTiter-Glo Microbial Cell Viability Assay (Promega, WI, USA). All experiments were repeated at least three times.

### ix Development of Antibiotic Resistance and Antibiotic Mutation Frequency

Resistance development was studied using serial passage experiments Cell suspensions were prepared at approximately 10⁶ CFU mL⁻¹ and treated with increasing concentrations of charged pi-conjugated compounds or antibiotics (linezolid, vancomycin, or ciprofloxacin) (FIGS. 10A-10C). After incubation at 30° C. or 37° C. (Table 2) for 18 h, cells growing at 0.5× MIC were collected and rechallenged with a range of charged pi-conjugated compound concentrations. This procedure was repeated for 30 days. Samples from each cycle were stored at −80° C. in 25% glycerol for later evaluation of the mutation frequency.

Mutation frequency was determined using rifampin resistance as a marker. The stored cells were revived overnight in LB broth and then plated on LB agar with and without rifampin (100 mg mL⁻¹) (Schaaff et al., 2002). The mutation frequency was calculated as the ratio of rifampin-resistant colonies to the total number of colonies.

To estimate the occurrence of spontaneous charged pi-conjugated compound-resistant mutants, S. aureus cells cultured overnight were resuspended in PBS to yield 10¹⁰ cells mL⁻¹ and then plated on both selective (charged pi-conjugated compound-containing) and non-selective LB agar plates. After incubation at 37° C. for 24-48 hours, colonies were counted, and the frequency of charged pi-conjugated compound-resistant mutants was calculated. All experiments were repeated at least three times.

### x Influence of Physicochemical Factors on the Antibacterial Activity of Charged Pi-Conjugated Compounds

The antibacterial activity of charged pi-conjugated compounds was evaluated under different environmental conditions, specifically temperature, pH, serum concentration, NaCl concentration, calcium concentration, and inoculum size. The effects of temperature were studied by performing time-kill experiments at both 25° C. and 37° C., as previously described. The effects of pH were determined using CA-MHB buffered at various pH values (5-9). The role of serum was investigated by supplementing CA-MHB with various concentrations of serum (0-50% v/v). The effects of NaCl and calcium were studied by supplementing MHB with different concentrations of NaCl or CaCl₂). The effect of bacterial population size was studied by using suspensions with variable cell densities.

### xi RNA Sequencing

Three distinct S. aureus colonies were cultured in CA-MHB and grown at 37° C. with agitation to mid-log phase, as previously described. Cells were treated with 0.5×MIC of charged pi-conjugated compound or 1% DMSO for 8 h. They were then filtered and fixed with RNA protect (QIAGEN, Hilden, Germany). RNA extraction was performed using the RNeasy Mini Kit (QIAGEN, Hilden, Germany) (Santos et al., 2022). RNA-seq and data processing were performed by Azenta Life Sciences (Leipzig, Germany). Sequencing libraries were prepared using the TruSeq Stranded Total RNA Kit with Ribo-Zero Plus IRNA Depletion and sequenced on an Illumina NovaSeq 6000 platform. On average, 70 million reads were generated per sample. For data analysis, reads were trimmed and aligned to the S. aureus reference genome, and differentially expressed genes were identified. Gene annotations were based on the National Center for Biotechnology Information (NCBI) database. Functional enrichment analysis of the differentially expressed genes was performed using Panther (https://pantherdb.org/).

### xii Membrane Potential

Overnight cultures of S. aureus were diluted 1:100 in fresh Mueller-Hinton broth and grown until they reached mid-logarithmic phase (OD₆₀₀ 0.4-0.6). Cells were then harvested by centrifugation, rinsed with phosphate-buffered saline (PBS), and resuspended in PBS (with glucose) to an OD₆₀₀ of 0.1 to which increasing concentrations of charged pi-conjugated compounds were added. Carbonyl cyanide m-chlorophenylhydrazone (CCCP) at a concentration of 10 μM was used as a positive control because it is a proton ionophore that collapses membrane potential.

After 6 h of treatment, the fluorescent probe DiBAC4 (3) was added at 1 μg mL⁻¹ (final concentration) (Clementi et al., 2012). The mixture was kept in the dark and incubated for an additional 15 min. The samples were transferred to a black 96-well microplate, and fluorescence was measured using a microplate reader (BioTek Instruments Inc., Winooski, VT, USA) set to excitation/emission wavelengths of 495/516 nm. After subtracting the blank value, relative fluorescence units (RFUs) were recorded. All experiments were performed in triplicate.

xiii Intracellular and Extracellular ATP

Cell suspensions (approximately 10⁶ CFU mL⁻¹) of S. aureus were prepared as described above and treated with increasing concentrations of charged pi-conjugated compounds for 8 h. The cells were collected by centrifugation at 12,000×g for 10 min. Extracellular ATP was quantified in the supernatant. The pellet was sonicated (using a Misonix S-4000), and the resulting supernatant was used to measure intracellular ATP after centrifugation. The luminescence-based BacTiter-Glo Microbial Cell Viability Assay (Promega, WI, USA) was used for ATP quantification according to the manufacturer's instructions. Luminescence measurements were performed using a microplate reader (BioTek Instruments Inc., Winooski, VT, USA). ATP content was determined by linear regression based on an ATP standard curve. Both the supernatant and pellet were used to determine the protein concentration using the Pierce assay (Pierce BCA Protein Assay Kit, Thermo Fisher Scientific, MA, USA). ATP content was then normalized against the protein concentration for each sample. All experiments were performed in triplicate.

### xiv Checkerboard Assays

The interactions between charged pi-conjugated compounds and conventional antibiotics and between different charged pi-conjugated compounds were studied using a modified microdilution checkerboard assay in an 8×8-well format (Santos et al., 2022). Bacterial cell suspensions (approximately 10⁶ CFU mL⁻¹) were prepared as previously described and treated with increasing concentrations of charged pi-conjugated compounds along the x-axis of the microplate. Next, increasing concentrations of antibiotics (or another charged pi-conjugated compound) in Mueller-Hinton broth were added along the y-axis of the microplate. After sealing and incubating the plates for 18 h at 37° C., bacterial growth was determined by measuring the OD₆₀₀, and the fractional inhibitory concentration index (FICI) was calculated. The fractional inhibitory concentration index (FICI) was determined as the sum of the MIC of charged pi-conjugated compounds and antibiotics when used in combination divided by their MIC when used alone. A FICI value≤0.5 indicates a synergistic interaction, a value between 0.5 and 4 indicates an additive interaction, and a value>4 indicates an antagonistic interaction (Odds 2003). All experiments were performed at least in triplicate. The interactions between charged pi-conjugated compounds and conventional antibiotics or between different charged pi-conjugated compounds were further characterized in time-kill experiments, as previously described.

### xv Toxicity Profiling and Therapeutic Index Calculation

The biocompatibility of charged pi-conjugated compounds with mammalian cell lines was assessed by determining their impact on the mitochondrial activity of human alveolar basal epithelial adenocarcinoma cells (A549) and human embryonic kidney cells (HEK293T) cells, using the Presto Blue assay according to the manufacturer's protocol. Mammalian cell lines obtained from ATCC were cultured in DMEM. The cells were treated with various concentrations of charged pi-conjugated compounds. After 8 h, Presto Blue cell viability reagent (Thermo Fisher Scientific, Waltham, MA, USA) was added to the cells, and after 2 h of incubation, fluorescence was measured using a microplate reader (BioTek Instruments Inc., Winooski, VT, USA). IC₅₀ values were derived from the resulting dose-response curves. The therapeutic index was calculated from the ratio between IC₅₀ and the MIC.

The biocompatibility of charged pi-conjugated compounds was further investigated in additional mammalian cell lines, specifically epithelial-like hepatocellular carcinoma cells (HepG2), a murine alveolar macrophage cell line (MHS), and the HEK293 variants HEK-Blue™ TLR and HEK-Blue™ NOD, as well as A549 cells and wild-type HEK293T, by quantifying ATP levels. Mammalian cells were treated with increasing concentrations of various molecules for 8 hours. Cell viability was then determined using the CellTiter-Glo™ Luminescent Cell Viability Assay (Promega, WI, USA) to quantify intracellular ATP levels. The concentration of the molecule that reduced cell viability by 50% (i.e., the IC₅₀) was determined.

Finally, biocompatibility was also assessed by measuring the impact of charged pi-conjugated compounds on the replicative capacity of mammalian cells using the clonogenic assay. Briefly, cells were initially cultured under standard conditions until reaching a confluency of ~80%, after which they were collected and treated with increasing concentrations of various charged pi-conjugated compounds for 8 hours. Following treatment, cells were inoculated into fresh media and allowed to grow for 5 days. During incubation, the culture medium was refreshed every 48 h. Post-incubation, the media was carefully aspirated and colonies were fixed with a mixture of methanol and acetic acid (3:1, v/v). Following removal of the fixative, cells were stained with 1% crystal violet (w/v).

### xvi Animal Studies

Animal studies were conducted in the invertebrate model G. mellonella (Santos et al., 2022). Larvae were purchased from a commercial source at a stage in their life cycle where they did not need to be fed. The larvae were sorted into Petri dishes lined with Whatman filter paper (Thermo Fisher Scientific, Pittsburgh, PA, USA) and stored at 4° C. until use.

Working solutions of charged pi-conjugated compounds were prepared in sterile phosphate-buffered saline (PBS) at increasing concentrations. For the in vivo biocompatibility assessment, cohorts of nine larvae were injected with 10 μL of each charged pi-conjugated compound preparation into the hemocoel via the last left proleg using a Hamilton syringe and needle. Injection of 1% DMSO (in PBS) served as a negative control. The injected larvae were incubated at 37° C., and survival was monitored afterward.

To evaluate the in vivo anti-infective activity of charged pi-conjugated compounds, MRSA USA300 was cultured overnight in tryptic soy broth and diluted to ~2×10⁷ CFU mL⁻¹ in sterile phosphate-buffered saline based on OD₆₀₀. Cohorts of five G. mellonella larvae (~300 mg each) were injected with 10 μL of the diluted bacterial suspension (2×10⁵ CFU mL⁻¹) via the last left proleg, following larvae sterilization with 70% ethanol. One hour post-infection, the larvae were injected with increasing concentrations of charged pi-conjugated compounds or 1% DMSO (in PBS) as a negative control. Uninfected larvae were also used as controls. Larvae were incubated at 37° C., and survival was monitored afterward. Death was defined by complete melanization of the larval body and lack of movement in response to touch.

xvii Statistical Analysis

Unless otherwise mentioned, the arithmetic mean and standard error of the mean across multiple biological and technical replicas were used as measures of center and spread. The number of replicates for each experiment type is included in the respective figure legends, where appropriate. Unless otherwise noted, all statistical analyses were performed using GraphPad Prism 8.0 (San Diego, CA, USA). When appropriate, the data were min-max normalized. Depending on the sample size, data normality was assessed using the Anderson-Darling normality test, D'Agostino-Pearson omnibus normality test, Shapiro-Wilk normality test, or Kolmogorov-Smirnov normality test with Dallal-Wilkinson-Lilliefor's test for P values. Comparisons between two groups were conducted using a t test for parametric data or a Mann-Whitney U test for nonparametric data. Multiple group comparisons were performed using analysis of variance (ANOVA) or the Kruskal-Wallis test with Dunn's multiple comparisons test. The Mantel-Cox test was used to determine statistical significance in the G. mellonella survival assays. Statistical significance was set at P<0.05. Where appropriate, asterisks are used to denote the significance of differences. *P<0.05, **P<0.01, ***P<0.001, ****P<0.0001. Unless otherwise noted, all figures were generated using GraphPad Prism 8.0 (San Diego, CA, USA).

### Example 3—Charged Pi-Conjugated Compounds for the Treatment of Cancer

i. Cytotoxicity is Dependent on the Time of Exposure and Concentration: Quantification by Crystal Violet Assay

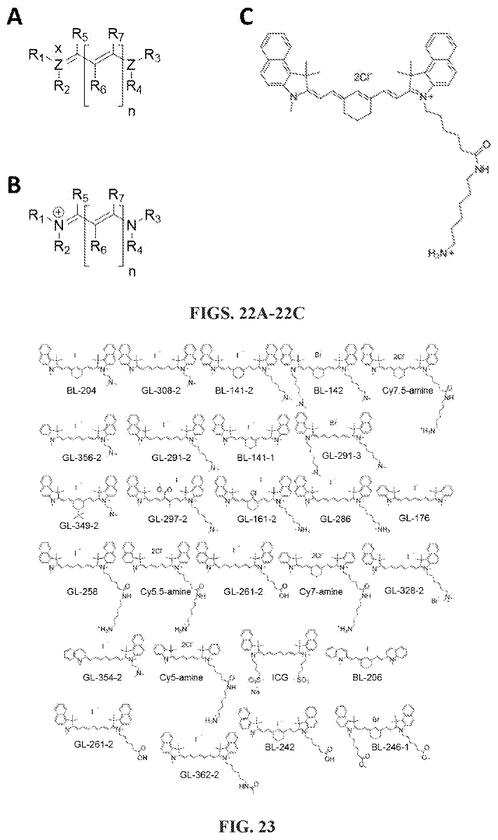

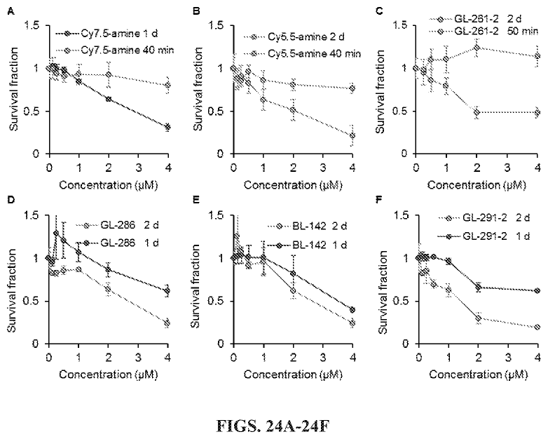

Relevant structures to the experiments described below and elsewhere in this Example are shown in FIGS. 22A-22C and FIG. 23. Most of the charged pi-conjugated compounds are aminocyanines which are functionalized with alkyl amine group. The charged pi-conjugated compounds were added to cell cultures of A375 cancer cells (human melanoma) and their cytotoxicity was quantified by the crystal violet test and/or clonogenic assays for cell viability. Charged pi-conjugated compounds (Cy7.5-amine, Cy5.5-amine, and GL-261-2) were safe and showed low cytotoxicity when exposed to cancer cells for a short period of time (for example: ~30-50 min) as shown in FIGS. 24A-24C. In the same FIGS. 24A-C, it can be observed that when the cells were expose to charged pi-conjugated compounds for longer time (for example: 1 day or 2 days), the charged pi-conjugated compounds showed cytotoxicity against A375 cells. The longer the exposure the most cytotoxic the charged pi-conjugated compounds are towards A375 cells. In FIGS. 24D-F, other charged pi-conjugated compounds (GL-286, BL-142, and GL-291-2) showed cytotoxicity toward A375 cells. The same behavior is observed: the longer the exposure, the more cytotoxic. All molecules presented here are aminocyanines except GL-261-2 which is a carboxyl-cyanine. Surprisingly, GL-261-2 has a protective effect toward A375 cells when used at short exposure time (~50 min) even at relatively high concentrations (1-4 μM). Also, it was observed that some aminocyanines such as GL-286 can increase the cell viability for short incubation time (1 day in contrast to 2 days) and relatively low concentrations below 1 μM. This was the first time a therapeutic window of concentration that can be tuned to selectively kill cancer and/or protect cells was suspected. In finding this therapeutic window concentration and time of exposure are important variables that should be fine tuned.

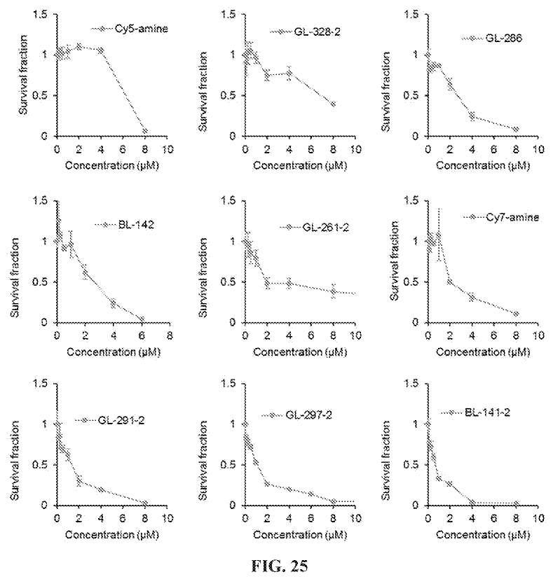

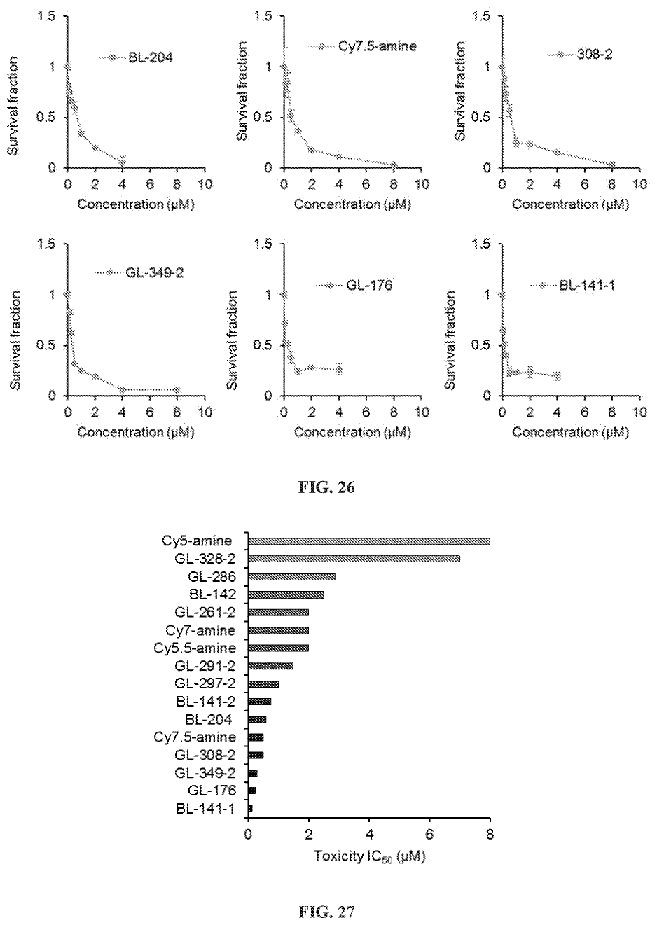

Based on the finding described above, a larger set of charged pi-conjugated compounds was evaluated for cytotoxicity against A375 cancer cells as shown in FIG. 25 and FIG. 26. The results of these studies are summarized in FIG. 27 and Table 3.

TABLE 3
Cytotoxicity of charged pi-conjugated compounds
to A375 cancer cells (melanoma).

| Molecule ID | IC₅₀ (μM) |
|---|---|
| BL-141-1 | 0.125 |
| GL-176 | 0.25 |
| GL-349-2 | 0.3 |
| GL-308-2 | 0.5 |
| Cy7.5-amine | 0.5 |
| BL-204 | 0.6 |
| BL-141-2 | 0.75 |
| GL-297-2 | 1 |
| GL-291-2 | 1.5 |
| Cy5.5-amine | 2 |
| Cy7-amine | 2 |
| GL-261-2 | 2 |
| BL-142 | 2.5 |
| GL-286 | 2.875 |
| GL-328-2 | 7 |
| Cy5-amine | 8 |

The results shown in Table 3 demonstrate very potent cytotoxic properties of charged pi-conjugated compounds against A375 cells with IC₅₀ in the order of 0.125-0.5 μM. The IC₅₀ is the concentration needed to inhibit the cell viability by 50%. These molecules in FIG. 25 and FIG. 26 were evaluated by crystal violet assay and using 2 days of exposure. In summary, Cy7.5-amine and GL-308-2 showed a potent cytotoxicity at IC₅₀=0.5 μM in A375 cells when exposed for 2 days. Molecules BL-141-1 and GL-176 were the most cytotoxic charged pi-conjugated compounds with IC₅₀=0.125 μM (BL-141-1) and IC₅₀=0.25 μM (GL-176).

j. Cytotoxicity Quantification by Clonogenic Assay

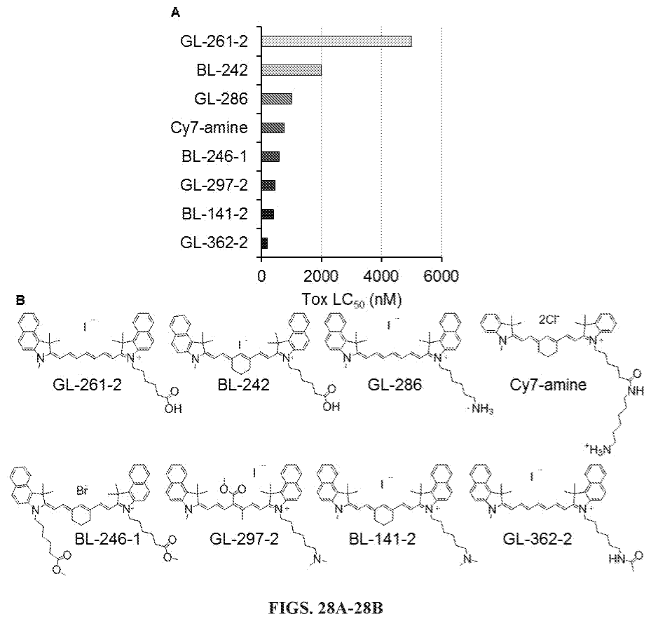

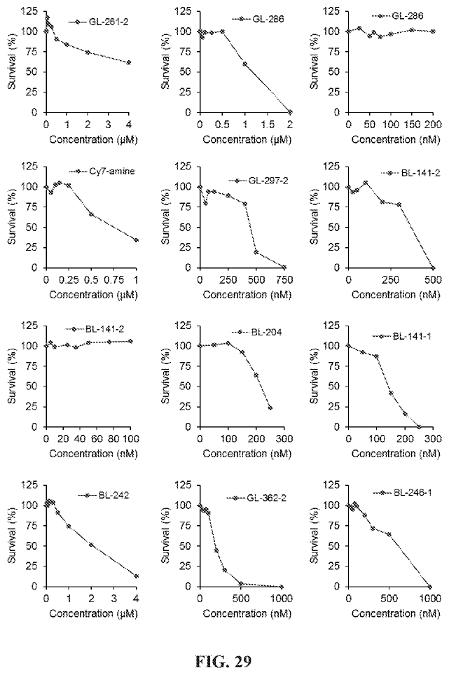

More cytotoxicity tests were conducted in some additional molecules and using clonogenic assay for cell viability (FIG. 28). A375 cells were exposed to charged pi-conjugated compounds for 7 hours, in contrast to the previously presented assays which were conducted with crystal violet assay and 2 days of exposure. The data points showing the cytotoxicity quantification for each individual molecule are presented in FIG. 29, with resulting IC₅₀ values summarized in Table 4.

TABLE 4
Cytotoxicity of charged pi-conjugated compounds
to A375 cancer cells (melanoma).

| Molecule ID | IC₅₀ (μM) |
|---|---|
| BL-141-1 | 0.14 |
| GL-362-2 | 0.19 |
| BL-204 | 0.22 |
| BL-141-2 | 0.4 |
| GL-297-2 | 0.45 |
| BL-246-1 | 0.6 |
| Cy7-amine | 0.75 |
| GL-286 | 1.0 |
| BL-242 | 2.0 |
| GL-261-2 | 5.0 |

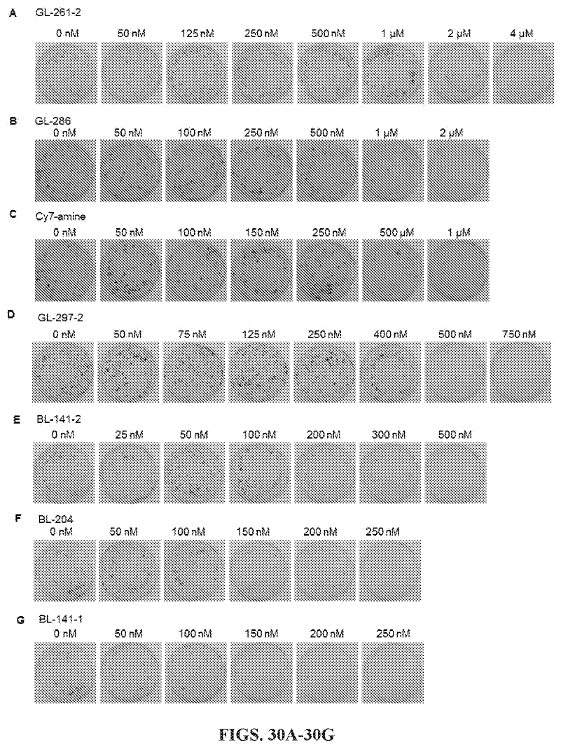

According to the results of the clonogenic assay, BL-141-1 (IC₅₀=0.14 μM) is the most toxic charged pi-conjugated compound in the group, followed by GL-362-2 (IC₅₀=0.19 μM) as shown in Table 4. FIG. 30 and FIG. 31 show pictures from the clonogenic assay. It is observed that death cancer cells are not able to form colonies at the lethal concentrations and confirms the high efficacy of these charged pi-conjugated compounds to eradicate cancer cells.

k. Dose Window for Selectively Killing Cancer Cells

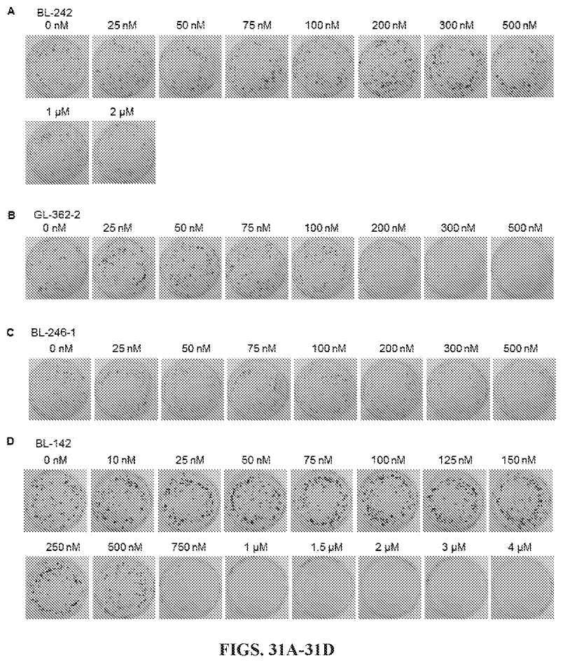

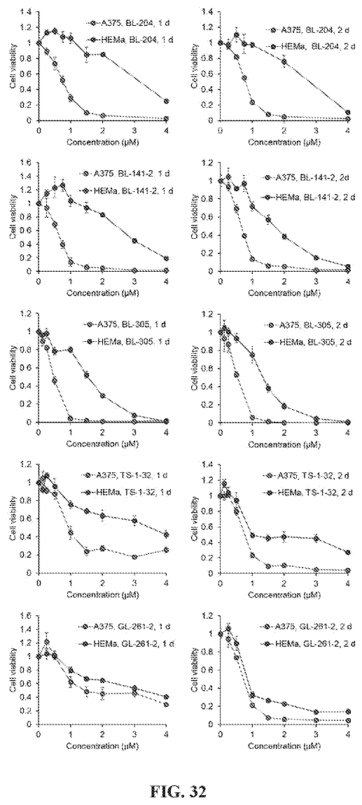

Charged pi-conjugated compounds such as BL-204, BL-141-2 and BL-304 were shown to be selective to specifically kill cancer cells (A375) but safe for normal skin melanocyte cells (HEMa) at 1 μM concentration (FIG. 32). Charged pi-conjugated compounds, therefore, can have a protective effect at very low concentrations (<0.5 μM) for normal skin melanocytes, are toxic selectively to cancer at relatively mild concentration (~0.5-1 μM), and toxic to healthy and cancer cells at relatively high concentrations>2 μM. This characterization is accurate for long exposures (~24 h) and in 200,000 cells/mL. The limits of toxic concentration (referenced herein as a therapeutic window) can vary with the exposure time and total cell number present in the test. For example, 1 μM concentration of charged pi-conjugated compounds are safe when used at 30 min exposure in the cell culture at 200,000 cells/mL. However, about higher than 2 μM cyanine becomes toxic (~8 μM) even at short exposure time (~30 min) for 200,000 cells/mL. In contrast, for a solid tumor this lethal concentration is expected to be higher since hundreds of millions of cells are contained in a small solid tumor. The dose may be calibrated and normalized by the total number of cells. The correct therapeutic window to kill cancer cells should be expressed in μg of charged pi-conjugated compound per 1 million of cells or ug of charged pi-conjugated compound per gram of cells. In summary, the same molecule is demonstrated to have agonist and antagonist effect at different concentration and depending on the cell type. Interestingly, the charged pi-conjugated compounds BL-204 and BL-141-2 increased the cell viability of normal skin melanocyte cells. Therefore, the presently disclosed charged pi-conjugated compounds may be useful in a clinical application in patients where the charged pi-conjugated compounds could protect the normal tissue and selectively cause toxicity to cancer cells.

l. Charged Pi-Conjugated Compound Therapy in Murine Tumors

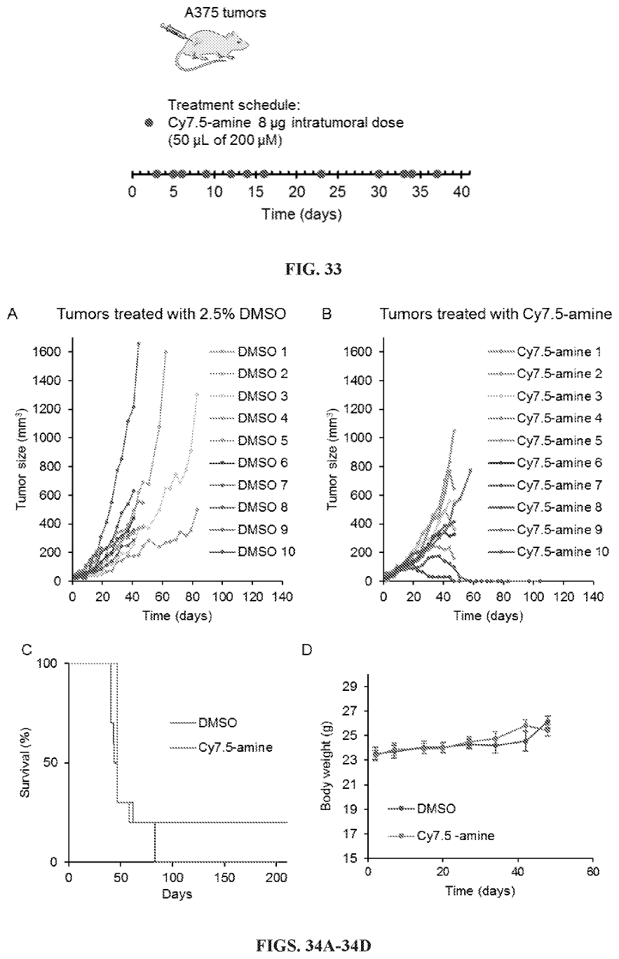

Then we applied Cy7.5-amine (FIG. 22C) by intratumoral injection into A375 tumors as described in FIG. 33. The results are summarized in FIG. 34. Cy7.5-amine was observed to cause a loss of 20% of A375 tumors and resulting in 2 out of 10 mice free of cancer even after 7 months of the study (FIG. 34C). In addition, the Cy7.5-amine showed no sign of toxicity to the mice as shown by no effect in the body weight in FIG. 34D relative to the control. This is a promising treatment for safe and selective tumor remission in cancer patients.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the disclosure as defined by the appended claims.

### VI. REFERENCES

The following references, to the extent that they provide exemplary procedural or other details supplementary to those set forth herein, are specifically incorporated herein by reference:
Adam et al., BMC Evol. Biol. 8, 52 (2008).
Ayala Orozco et al., ACS Appl. Mater. Interfaces. 12, 410-417 (2020).
Ayala-Orozco et al., bioRxiv (2023).
Avire et al., Pathogens 2021, 10 (2), 248.
Batt et al., Biochem. J. 477, 1983-2006 (2020).
Bumah et al., Photomed. Laser Surg. 2013, 31 (11), 547-553.
Clementi et al., J. Vis. Exp., e51008 (2014).
Dan et al., Proc. Natl. Acad. Sci. U.S.A 118 (2021).
Gonzales et al., Lancet 2001, 357 (9263), 1179.
Hajdu et al., Antimicrob. Agents Chemother. 54, 4078-4084 (2010).
Huemer et al., EMBO Rep. 21, e51034 (2020). Ikuta et al., Lancet 2022.
Kincses et al., Antibiot. (Basel, Switzerland). 10 (2021).
Kaplan et al., J. Chemother. 25, 18-25 (2013).
Lange et al., Pharmaceutics. 13 (2021).
Li, et al., Front. Microbiol. 12, 705326 (2021).
Maitra et al., FEMS Microbiol. Rev. 43, 548-575 (2019).
Martinez & Baquero, Antimicrob. Agents Chemother. 44, 1771-1777 (2000).
Moellering et al., Ann. Intern. Med. 2003, 138 (2), 135-142.
Morrissey & George, J. Antimicrob. Chemother. 43 (1999), pp. 423-425.
Murray et al., Lancet 2022, 399 (10325), 629-655.
Niculescu & Grumezescu, Appl. Sci. 11 (2021).
Odds, J. Antimicrob. Chemother. 52, 1 (2003).
Peterson et al., J. Clin. Microbiol. 2010, 48 (3), 683-689.
Prakash et al., Dye. Pigment. 211, 111053 (2023).
Weigel et al., Science 2003, 302 (5650), 1569-1571.
Smith et al., N. Engl. J. Med. 1999, 340 (7), 493-501.
Santos, et al., Adv. Sci., 2203242 (2022).
Santos et al., Adv. Sci. 10, 2205781 (2023).
Schaaff et al., Antimicrob. Agents Chemother. 46, 3540-3548 (2002).
Serrano et al., Antibiotics. 12, 505 (2023).
Silhavy, et al., Cold Spring Harb. Perspect. Biol. 2, a000414 (2010).
Silverman et al., Antimicrob. Agents Chemother. 47, 2538-2544 (2003).
Stewart & Costerton, Lancet. 358, 135-138 (2001).
Thomas et al., Int. Wound J. 9, 428-435 (2012).
Wenzel et al., MBio. 9 (2018).
Xie & Yang, Sci. Rep. 6, 20628 (2016).
Yoon et al., Biomed Res. Int. 2013, 274096 (2013).

What is claimed:

1. A method of killing bacteria by treatment with an extended conjugated compound of at least two double or triple bonds in conjugation that bears a charge in the conjugated chain.

2. The method of claim 1, wherein the bacteria are Gram-positive bacteria.

3. The method of claim 1, wherein the bacteria are Gram-negative bacteria.

4. A method of killing a cancer cell by treatment with an extended conjugated compound of at least two double or triple bonds in conjugation that bears a charge in the conjugated chain.

5. A method of killing a fungal cell by treatment with an extended conjugated compound of at least two double or triple bonds in conjugation that bears a charge in the conjugated chain.

6. The method according to any one of claims 1-5, wherein the extended conjugated compound comprises a positive charge.

7. The method according to any one of claims 1-6, wherein the conjugated compound is further defined by the formula: 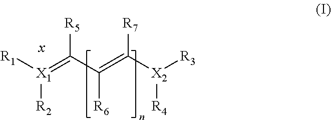
wherein:
x is a positive or negative charge;
n is an integer from 2 to 100; wherein the variables on each independent n is independently selected;
X₁ and X₂ are each independently a heteroatom selected from O, N, S, B, P, Ge, As, or Se; and
R₁, R₂, R₃, R₄, R₅, R₆, and R₇ are each independently hydrogen, alkyl_{(C≤18)}, alkenyl_{(C≤18)}, alkynyl_{(C≤18)}, aryl_{(C≤18)}, aralkyl_{(C≤18)}, heteroaryl_{(C≤18)}, heterocycloalkyl_{(C≤18)}, or a substituted version of any of these groups; or
R₁ and R₂, R₁ and R₅, R₂ and R₅, R₃ and R₄, R₃ and R₇, and R₄ and R₇ are taken together to form one, two, three, four, five, or six aliphatic or aromatic rings; comprising at least three carbon atoms and no more than 36 carbon atoms; optionally comprising one, two, three, four, or five nitrogen, sulfur, or oxygen atom.

8. The method of claim 7, wherein the compound is further defined as: 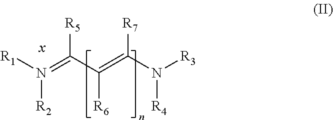
wherein:
x is a positive charge;
n is an integer from 0 to 100; wherein the variables on each independent n is independently selected;
each R₁, R₂, R₃, R₄, R₅, R₆, and R₇ are each independently hydrogen, alkyl_{(C≤18)}, alkenyl_{(C≤18)}, alkynyl_{(C≤18)}, aryl_{(C≤18)}, aralkyl_{(C≤18)}, heteroaryl_{(C≤18)}, heterocycloalkyl_{(C≤18)}, or a substituted version of any of these groups; or
each R₁, R₂, R₃, R₄, R₅, R₆, and R₇ are each independently a cell membrane targeting moiety, wherein the cell-targeting moiety optionally comprises a linker; or
each R₁ and R₂, R₁ and R₅, R₂ and R₅, R₃ and R₄, R₃ and R₇, R₄ and R₇, and R₅ and R₇ are taken together and each independently form one, two, three, four, five, or six aliphatic or aromatic rings; comprising at least three carbon atoms and no more than 36 carbon atoms; optionally comprising one, two, three, four, or five nitrogen, sulfur, or oxygen atom.

9. The method of claim 7, wherein X₁ is N or X₂ is N.

10. The method according to any one of claims 7-9, wherein R₁ is taken together with R₅ to form one, two, three, four, or five rings.

11. The method according to any one of claims 7-10, wherein R₃ is taken together with R₇ to form one, two, three, four, or five rings.

12. The method according to any one of claims 7-11, wherein R₂ or R₄ is alkyl_{(C≤18)} or substituted alkyl_{(C≤18)}.

13. The method according to any one of claims 7-12, wherein R₂ is methyl.

14. The method according to any one of claims 7-13, wherein R₄ is methyl.

15. The method according to any one of claims 7-9 and 12-14, wherein R₅ and R₇ are taken together and form a single ring, wherein the single ring is a five, six, or seven-membered ring.

16. The method according to any one of claims 7-15, wherein n is an integer selected from 2, 3, 4, or 5.

17. The method according to any one of claims 7-13, 15, and 16, wherein R₄ is a cell targeting moiety with a linker.

18. The method according to any one of claims 1-17, wherein the compound is further defined as:

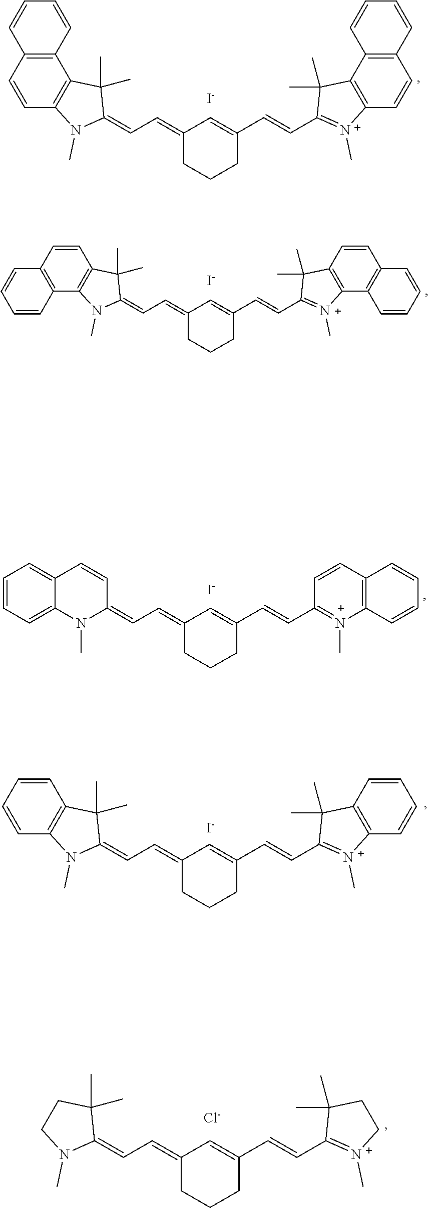

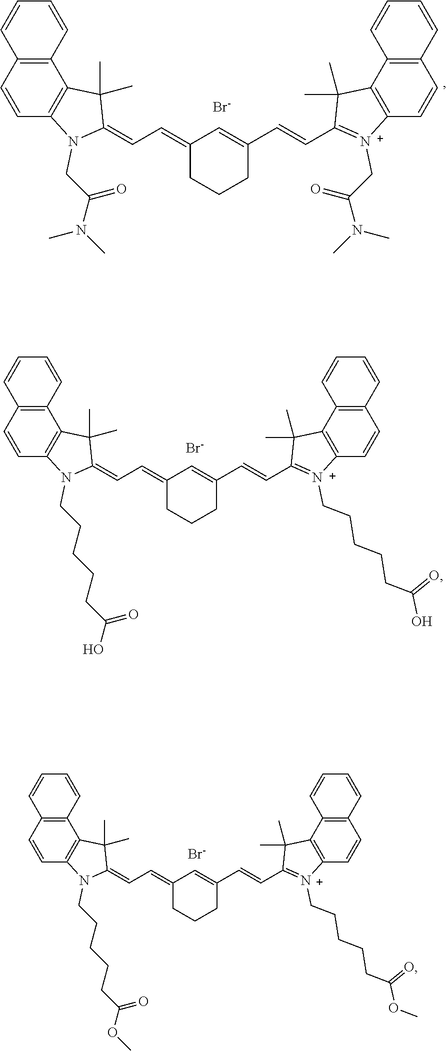 [SMILES: CN(C)C(=O)CN1/C(=C/C=C2C=C(/C=C/C3=[N+](CC(=O)N(C)C)c4ccc5ccccc5c4C3(C)C)CCC/2)C(C)(C)c2c1ccc1ccccc21 ; [Br-] ; CC1(C)C(/C=C/C2=CC(=C/C=C3/N(CCCCCC(=O)O)c4ccc5ccccc5c4C3(C)C)/CCC2)=[N+](CCCCCC(=O)O)c2ccc3ccccc3c21 ; [Br-] ; COC(=O)CCCCCN1/C(=C/C=C2C=C(/C=C/C3=[N+](CCCCCC(=O)OC)c4ccc5ccccc5c4C3(C)C)CCC/2)C(C)(C)c2c1ccc1ccccc21 ; [Br-]]

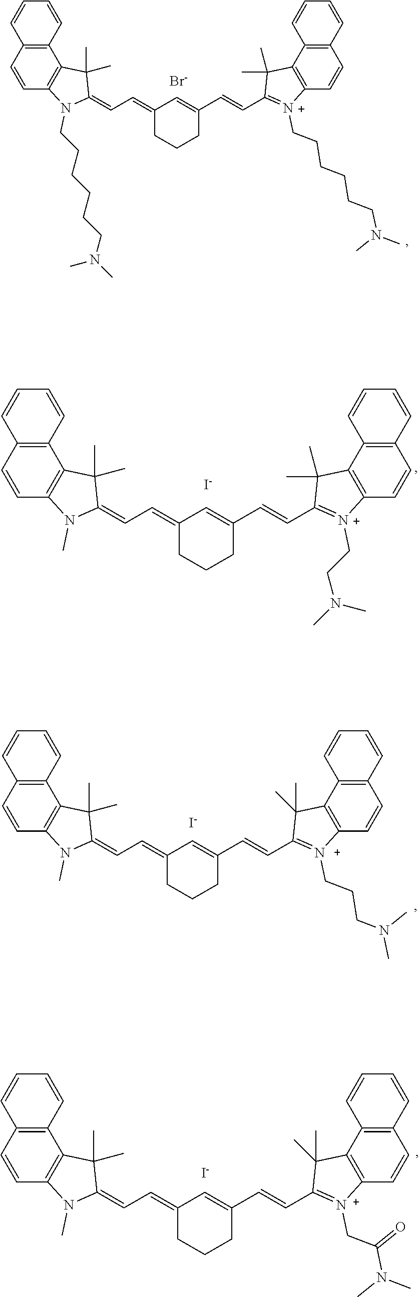

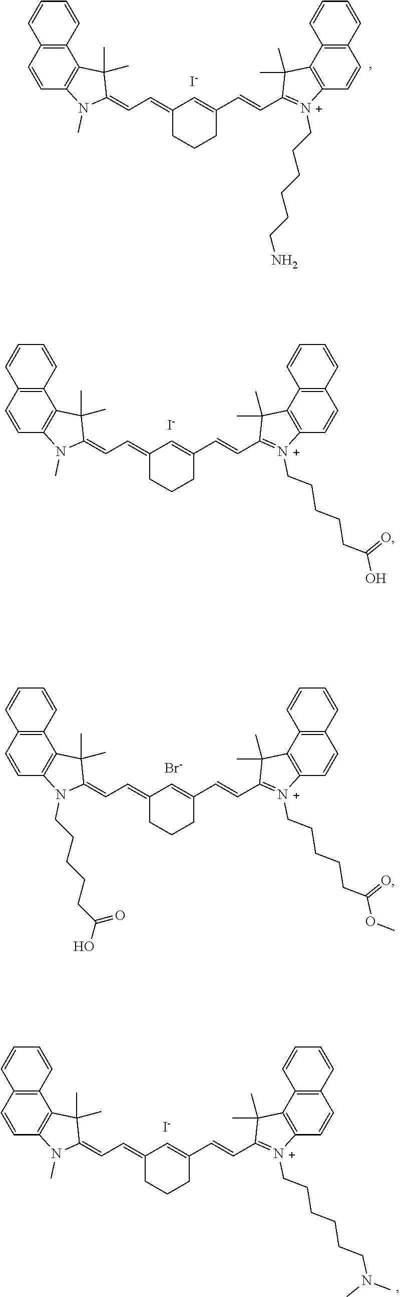

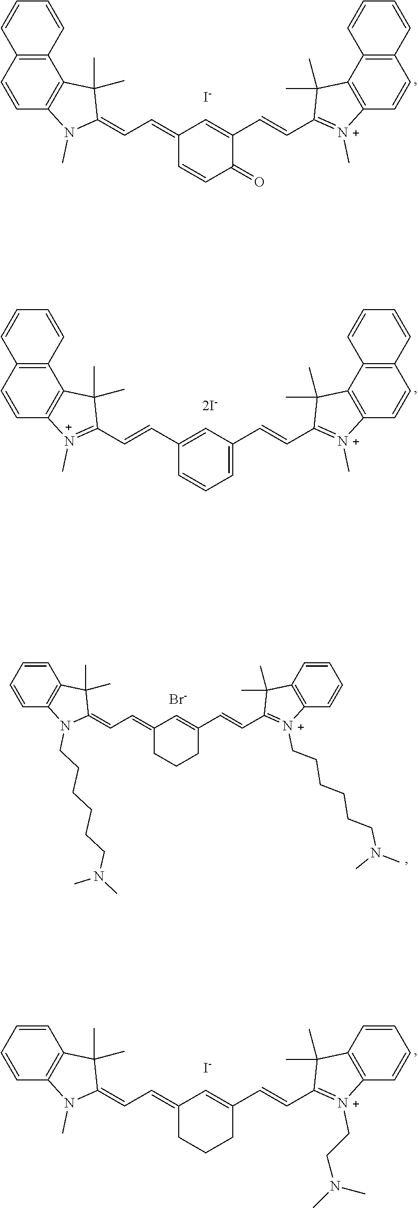

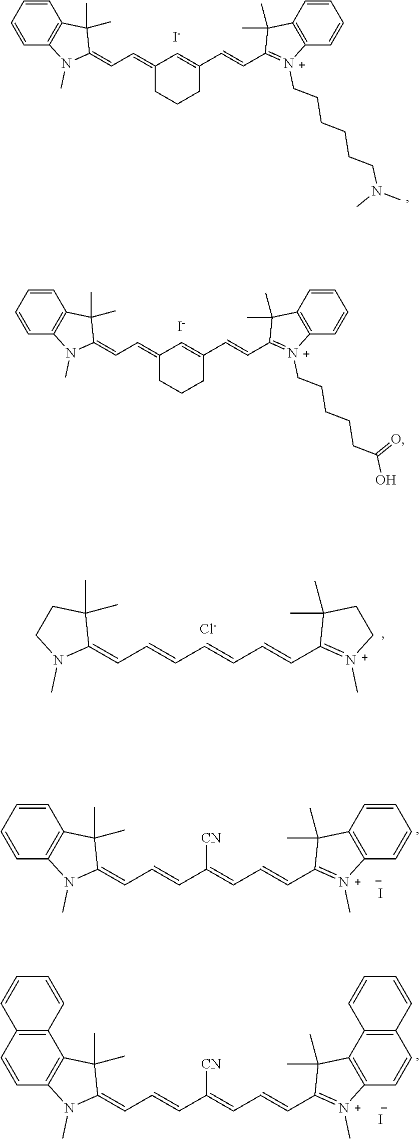

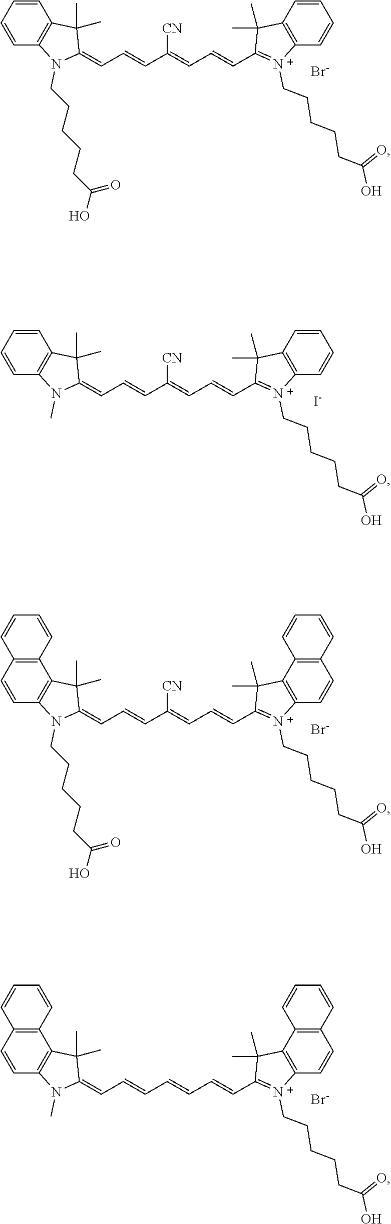

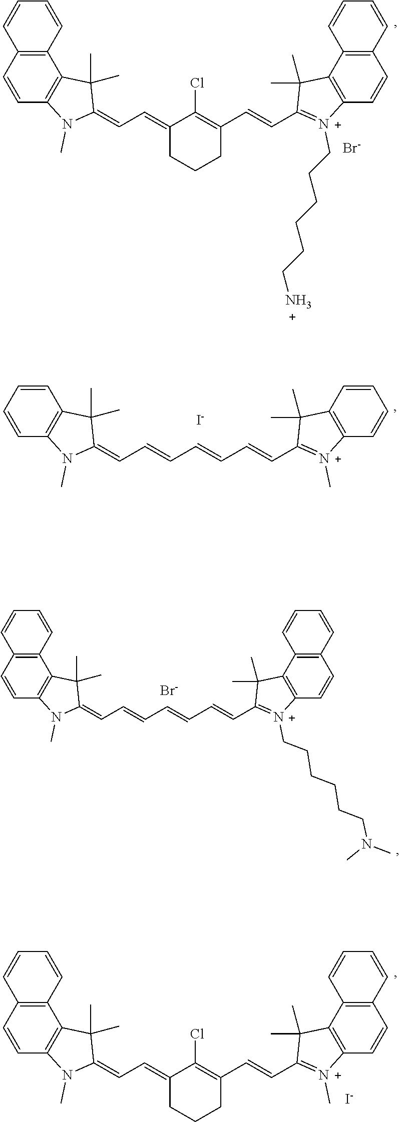

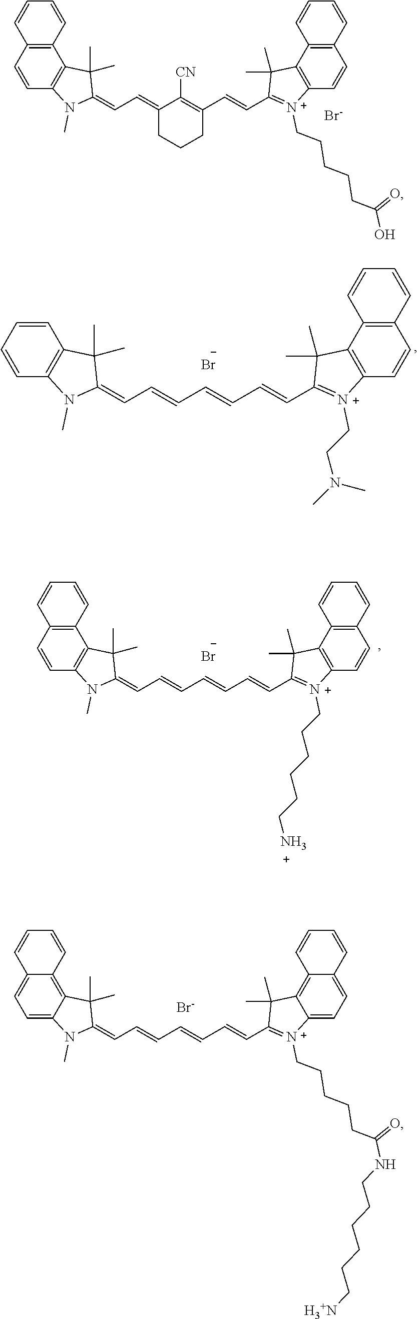

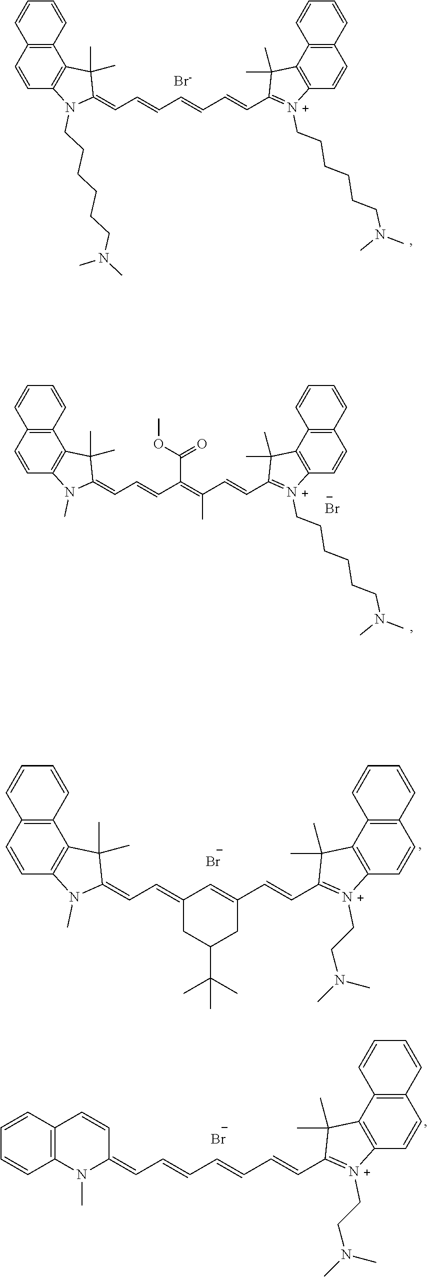

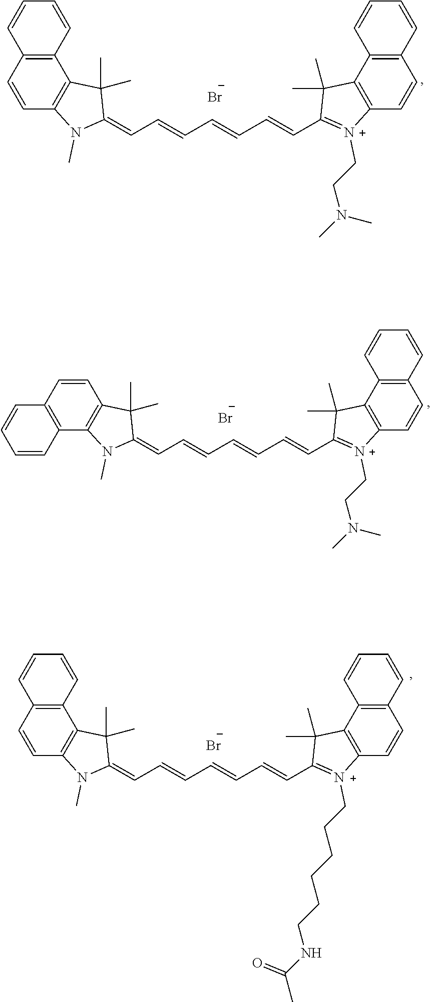 [SMILES: CN(C)CC[N+]1=C(/C=C/C=C/C=C/C=C2/N(C)c3ccc4ccccc4c3C2(C)C)C(C)(C)c2c1ccc1ccccc21 ; [Br-] ; CN(C)CC[N+]1=C(/C=C/C=C/C=C/C=C2/N(C)c3c(ccc4ccccc34)C2(C)C)C(C)(C)c2c1ccc1ccccc21 ; [Br-] ; CC(=O)NCCCCCC[N+]1=C(/C=C/C=C/C=C/C=C2/N(C)c3ccc4ccccc4c3C2(C)C)C(C)(C)c2c1ccc1ccccc21 ; [Br-]]

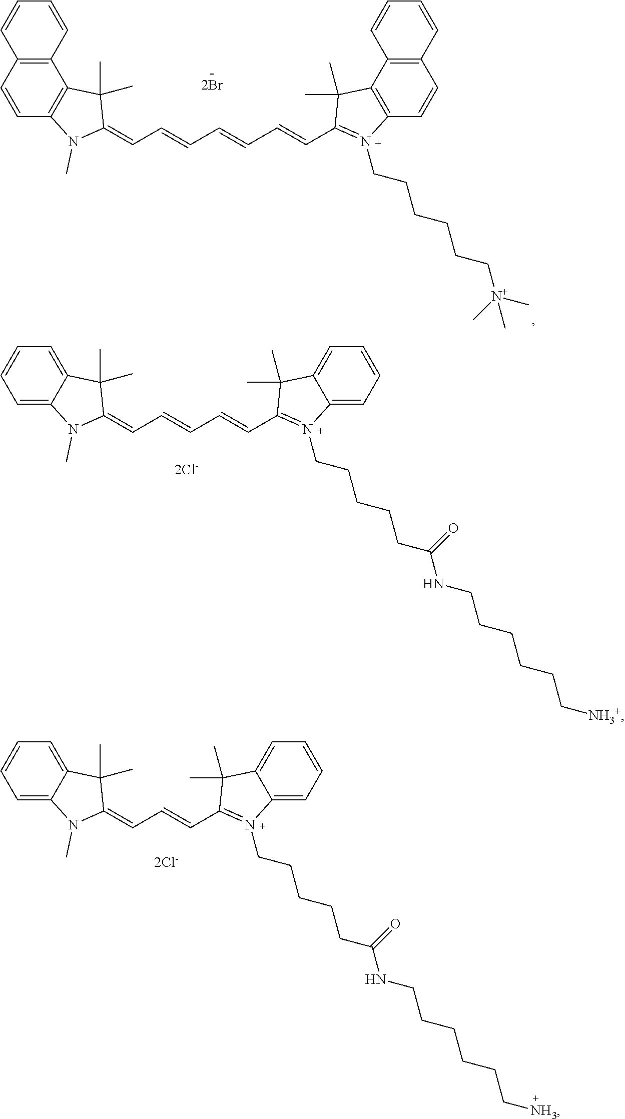

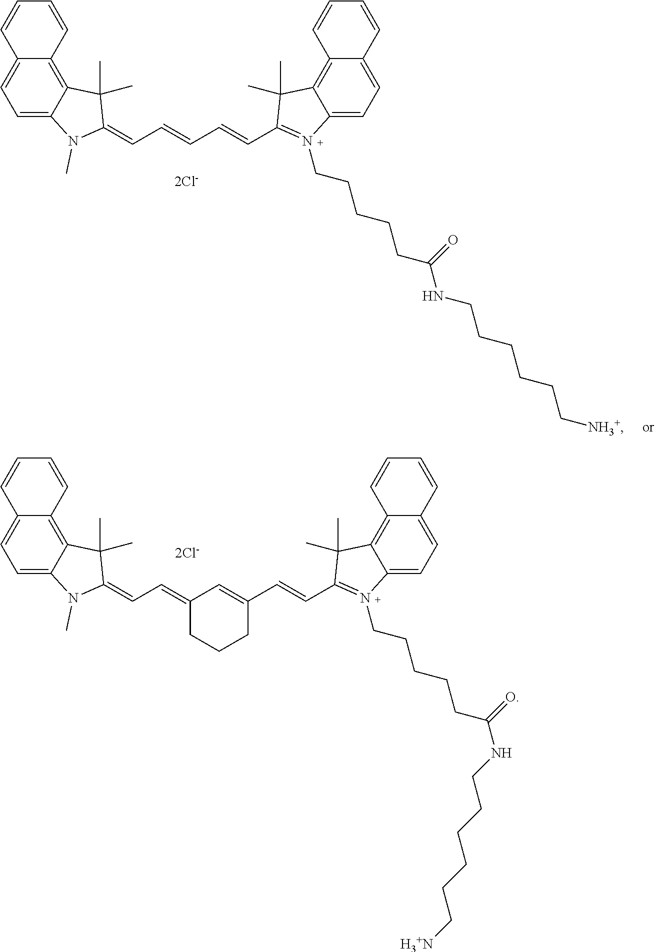

19. The method according to any one of claims 1-18, wherein the method is sufficient to treat or prevent a disease or disorder in a patient caused by either a bacterium, a cancer cell, or a fungal cell.

20. A method of forming a compound of formula (IV) 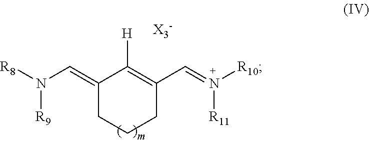
wherein:
m is 0, 1, 2, or 3;
X₃ is Br, Cl, I, or F; and
R₈, R₉, R₁₀, and R₁₁ are each independently hydrogen, alkyl_{(C≤18)}, alkenyl_{(C≤18)}, alkynyl_{(C≤18)}, aryl_{(C≤18)}, aralkyl_{(C≤18)}, heteroaryl_{(C≤18)}, heterocycloalkyl_{(C≤18)}, or a substituted version of any of these groups;
comprising the steps of:
(a) obtaining a compound of formula (III) 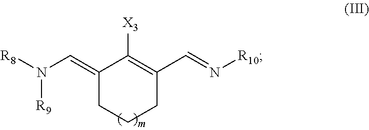
wherein:
m is 0, 1, 2, or 3;
X₃ is Br, Cl, I, or F; and
R₈, R₉, and R₁₀ are each independently hydrogen, alkyl_{(C=18)}, alkenyl_{(C≤18)}, alkynyl_{(C≤18)}, aryl_{(C≤18)}, aralkyl_{(C≤18)}, heteroaryl_{(C≤18)}, heterocycloalkyl_{(C≤18)}, or a substituted version of any of these groups; and
(b) reacting the compound of formula (III) with a catalyst and a base under conditions sufficient to form the compound of formula (IV).
